SEQUENCE LISTING (28 sequences)

SEQ ID NO: 1 - DNA, 18 bp, Artificial Sequence
  Forward Primer CD40L MND.GFP (HDR)
acctgtcagctcctttcc

SEQ ID NO: 2 - DNA, 22 bp, Artificial Sequence
  Forward Primer CD40L cDNA (HDR)
agtgtcttcgtcaacgtgacag

SEQ ID NO: 3 - DNA, 20 bp, Artificial Sequence
  Forward Primer ActB
actctgcaggttctatttgc

SEQ ID NO: 4 - DNA, 20 bp, Artificial Sequence
  Reverse Primer CD40L MND.GFP (HDR)
ggtccagatcctaagagagg

SEQ ID NO: 5 - DNA, 23 bp, Artificial Sequence
  Reverse Primer CD40L cDNA (HDR)
tccctgataaagtgcaatcatcc

SEQ ID NO: 6 - DNA, 20 bp, Artificial Sequence
  Reverse Primer ActB
aatgatctgaggagggaagg

SEQ ID NO: 7 - DNA, 20 bp, Artificial Sequence
  Probe CD40L MND.GFP (HDR)
tcaatccagcggaccttcct

SEQ ID NO: 8 - DNA, 20 bp, Artificial Sequence
  Probe CD40L cDNA (HDR)
tcctggttagttcttgccac

SEQ ID NO: 9 - DNA, 20 bp, Artificial Sequence
  Probe ActB
atcaaggtgggtgtctttcc

SEQ ID NO: 10 - protein, 30 aa, Artificial Sequence
  CD40LG TALEN forward (RVD sequence)
HDNGNGHDNGHDNINGNNHDNGNNHDHDHG

SEQ ID NO: 11 - protein, 26 aa, Artificial Sequence
  CD40LG TALEN reverse (RVD sequence)
NNNHNININGNGHDNGHDNGNGHDNG

SEQ ID NO: 12 - DNA, 20 bp, Artificial Sequence
  CRISPR GUIDE sequence
aaagttgaaatggtatcttc

SEQ ID NO: 13 - DNA, 12214 bp, Artificial Sequence
  CD40LG gene sequence
actttgacagtcttctcatgctgcctctgccaccttctctgccagaagataccatttcaactttaacacagcatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaaggtaagatgaaccacaagcctttattaactaaatttggggtccttactaattcataggttggttctacccaaatgatggatgatggtagaaaccaaatagaagaatggtcttgtggcataatgtttgttgcctagtcaatgaagtctcatattcttgtctctggttaggatcttgggatctggagtcagactgcctgggttcaaatcttggctctgcccataccatctctgttatcctggggcaagtgcctcagtttccacatctgagaaatggggatggtattggtgtccatttcatagattaagtgagtttagccttgtaaaaagcttaggagggggtctgatacatagtaagcactatgtacgcactagctataattatttgctaaagttctgctttaaaagtaagctatttttttatggagacagcttttttcttttaaatttccagctaggcaagaagagcgtcaatttgatctaaaatttcataatgcttcagattaacatagacatggataagtcccagaatttgcagtcttttagtaaaagtagcattttctgtgtaattcttcacaagcactgattgtagttgcaggatgctcagtctccctctgagatgttttacatttttaaatggttagacttgcaggaacaaaagagcagagtaacttagtaggctgttttgcattcttaggaaaagaaaaccatcaggacttattttgttttcatgtattttttcacttccactgaggagtataattggctggtgttgacaaaataccaatcatagatgtaaaggagaaagttgattagttttctggctgttcctaaaattctggatgcaggaactgtggctagaaagcatctggatgattgcactttatcagggatacttgagtgtcctctcttaggatctggacctagaattaatgtcatgagatttttctaacaggataaggtgaggtagtgagggctgaagtcatccactgggttatccaaatattaggtttcactgctgacaaaagagggggcttctggtctggttggttatttgtgtttggcctgatgtgctctgtcaatcaaatgtatggacataggcctagcttctaaaggggcaatagtgacctcagtggactgatatttaccgtactatttacatgtgctcttaattacagcagaagctgccagctaactgaatcttgttttgaatctaaaaaatctactcttaaagcaagaaaatggtataaaattagttgataatgcaagtgaattctgtacatttaattattctaagacattggaaaataaaatatcttgttactttgaggataaaagatgatttctttaaaaatgcaaatgttttctacaaatactaaagttaaaagggagagagatgtaattagaactcgttaactgacacattgcaaattaacttctttttataaagcactgcatcacaaacactaaaatgaagtgggcaaattagctctgcagaaaactattttctaggctgatgtttataatgaccaatcattactgaagcaatgagaaatgtgacaattacagaatattgctgctatagtatgttgaaaaaatatgcattttgtagtgaacatttagtagaatagctctgatttctacctggagtttctgataacatgacatcttaattgctgtcttttatagatttttaaactgcaaatacaaaatagcaatcagccaatataataacttattattctccatttatgcctgaaagtcctcctcttgttgatgccgtggaaatgaatgtagaggcagatatcattagctgtattctccttccgaatgacatttatcatatccttgttattccaaaatagatagaagatgaaaggaatcttcatgaagattttgtattcatgaaaacgatacagagatgcaacacaggagaaagatccttatccttactgaactgtgaggagattaaaagccagtttgaaggctttgtgaaggtaagcagcttaattactggtaaaagtgtcattgaaatattttactacatttgctagatcgggaaactgacaatgccaatgtttaaagattggttatagacacagacacacagacacacacacacatatatatgcatgcagatatacacacatacatgggtgtgtgtgtgggggttaaaaaaaaaaaacacaaagacactctctggggaaaatacacccttaggggcacagtcacacatatttgtcagcttacatatgcagctaccactaggcaaaatgatgaagtccaccaagcttggtttttgcattgctgtgtctccccatccaaaccttgatgctctcgcactggggacccagagtctgatccccatttcccagggaagcaatagccgtcaacagctgccgtggcagcaggccacaagtgaagggacacctgaagactggtaacagtctctggtgcttctctgatgatggaattttaggtgtcctgacagtgagatctttcccttttactggggagagaggtgcagggaataagtaatagacattctcagtgtcgctcaaaccagactccatataatatcacttgctcatgaagcccgcccactctatggccggtcatgaccagaggcacagagggttcaaagccttttagcccaccaggctggtagctagcatgaagtcactgcagtgactgtggcttataacagatacctaaaacaagaatttttagaacctttacattaattccatcatcacagacatagggtctaggggctctttctcctgaggcagaacatcaagagttctttctgcctatgtccctttcagaacactgagtcaaatacccttgggcctcggctcacttaggggtcatttctaggaggcagcactccacattgaggacagttctgggccaggtgggtgggtatctgggtaaaccaacaggaattagttctcacatatagatgatgtgtaatttaatgcaggcgtaaaagggttaagatcttatttctgatcttatttctgccctcctgtactgtcaccgaggtgccatttaattcattagtgaagactctaacagcttattcctgagtcacctacggagaacagaatgtggctcaaatccgctgcttgctttcaggttctttacactaatctaggctttagatgaaactcctaaaccctttctttgcaagactggccagctaggaaaatgatttgagtttcttcggttcttcgaggatttgggccagtattacagagtattggaagatgttaccagtttaaatgtgaataaaggcactttcaaaacaatggctaataatccaaataacagactgaatgtgcttggctatgtgactttgggtaaataacttcacctttctgggcctcagttttgtcatctataacatgagaagacagattatctgtaagggcactatcagctctgacattctacaattatgtgataagccttcagttccctccaatggcagtgagagtggcttgtcagtccccctcgtttcttacggagacttttacggttgaattgtcaattcctcacgtcattatttcaggttggctatgtatgtaaagctcccaaaatcagctaccgaggataggagtaaagaaaacagtcagtttggcctccctgcttatgcttgtatgaaaaaagtgacagctccaaagtttcatattcttaaaaggcagatcttctcaggcatgtcagccagggccccagggatctcctccttacatgcaactaaggaggctccttgtctctactgcagcaggtgtggaaccctagtcaacaccacctatacctaggattacgtacaatgagtagatacaaagtcctccagctacccaatcctcccccaatgacggatcccctttccaatacgctttcccccaaatttctcaccctaaaacaaaattcgagactttgaaaaaactcaataggacaattatagaatagctccagattagattcatattttcttagctaatgttagtaggctttctttccgggccacagtctggctgcacctaagcaacctcaagtttgaatttggagtctttgaatcaggtcttgatggggtcttagaagtcatcagatccaattctcaatccacaacttcagtcttctctccacctcctgactaagtggtcatccaatctctgtttgaacatctctagtgacaaggaactcattatctctggaggcaggtagcactaatctgtcattttgggggaaagatggtattcagggctcaagtgagggtaagcagaggtattattttgaatagtataatttcatattaaaacttacaacccaccacacctctgctagatgttcagttccatgattatttgcccaccaatgcctgcgatgcctttgagagagccaaagcatttctatttcaagttaaagggcaacctgtccatacctgccacatggaactcccactaagagagaaataacccattctggattttctgaaagtccactttaaaaagtatttcagttgaggtggggagtgaagcaagaaaaaaaaaaggctctggggagtgtggttgggcgaaagttcacggaaaggctaggctgggctcatgaaacacgagctttgctgacttcatgttttcatcttggccaggcctcaacaccaatgcaacaacttagcctaaaagtatctcaaccttgatcaccacactctactttttgaaaagacactaaatagtcatttgtttacttgtgatctcacaaacattttcctgtcaccacatcttcatagtgccgcgcttcagctcaaatggaaagttgaagctctggggcccatgtgagtgttctgaggctcaggttcccctggaggctctatgaactacgcccttaaatctggcaactgagctgggcctacagccagcactcaacagtgacagcacaaattccttctggaggaggaaataaaaggaagggtcctatagacaactgattccaggagtgggaaggagcacaggactttgattatcataagatgtgaaaatactactgtcttcttcccttgtgtgcagaggatagacagatggaattagctaagcccagcctatgaatgccatctcacagtttccactcttggtttaaacctcagcttctttgggtgacctcataatgaccagttaagccctccaggccttttgttcagtctctttaaaatggcagcaacagcctttatcatcttccaacctgtgttgatggaagttcctgttagcttctttaaatacctctagacttccttcagtttataagtgaaaagaaaccttttaagaagtgtcgcacttgcctttgaacatcaacaccattgggagatggcctgtgtttccgaaatgctgattattctaagtaaatacagtgcaactatcaataagagaatctcttcagcccattgaaagggatagcaaaattaaaaatgtctgagggtcttttcatagtctggcatttctccccaaggtcaaacttactattatcttttcctacaggatttcagaccaaatttattctaatagatacacaccatgctttatgtttaataatattccatataccagttcccagggtagaatcatctccccattcggcattatttgtcaatatctgtcaaagccaaggaggttgaggtcataggaagggtcaggatcacagcctctggtctggagagagcactggaatggagataataaggcctggattttacttccagattctcccctgggctttctgggttgttggctcatctgtcagatccatggactcccaattggcatgatggaattaatgacaggatctgagtctatatgataatcctcaccagaaacagacaacagagtaatgacagatgcaaaacgaatgataattttaaaaccccacagcagagcccctgtcaaaatgacctcttgcaatgcttcttattttaggatataatgttaaacaaagaggagacgaagaaagaaaacagctttgaaatgcaaaaaggtaggtttgctatttgctaatttctatgaatgcctaaaaactaaaaggaagctttaggctgatcatattgaacaacccagtgttgttgcatcagggaacttttagccctggaaataaaacaggaacacaattgtcaaattgacaccttctctggtccctgtgatttggaaagactttgtacatatatatttatgaaaaaaggatgtgttcctttaatgccgatgataccaaatctgaagaaatcccattatgttcaataccttaatagaagcaaccatacagcctgataccacctacagtggaataagaagacaggaaagtcatcatttggtaacagtggcattcatcactcattgataacagtttttcatggggcacagtggccggtggagcctctgggatcaaggagtgacaatgtcacagtgttctattatttgcccggttcttaaagtgagagcatcctgaacatctcagggttggaagagaacttgagagttctcaaatccagcaccatccccacaacaaaaatctccttcacaataacactgaccgtccagcctctgatcaaacatgtcgagggatgaggcaccttccacctcataaggcagcctgatccgtctttgaatggctctaataataccaagattactatactactccagagaagtctttcctcctcaagtcaaactttgttcctataatctccactcattggtcccagttctgctctttgaggccctagtaaacaaagtataattgctctcctacccagcagctgtccagatatggaagacagcaatcatggtggccaagccttgactgagctttttcttctccaggctaaagatccctgatgtcttccactgtttctcctatgaccctttccaggacctttcttctgccactcacctccttttcttggacacactaacgttttcctgttcttttagaatgtggcatcgcaaaccaatacaataatgcgtgaagtgacttcagcagcagattatgggaaagacggggtgttgttagagagaattttatatcacaaagttggtgaacatgatgttatggcttctgcaaatttaatacacacaaaaacatacatacatacagggatagagatactattttctgaggcaaagagagtactcagaccttgccttaactgttgttctggatactaaatggtcatccgacttccatgaaggttttatcttcagaatgactgcaagatatgttgagtaatagtaccacgctgtctgttaattacagagaaatctgaggaaacagtttatgtagatgctgcctagaagtcttcagggaaatgataatattaaccaaactggtcatttaggtcatgcaatttaactcaacatttatagggcacttacaaagtgcccaatatcaggctcataactggacaaaaagaaacttccacacagtctctgcccttagaagattgacacatctcattagggagcagggctttaacacaagaaataattaaagacagatacaatagttcagccagttgcttgaccaattcagaaaccataagaatcttactaagtgtgcagactttggagcccactaaaatccccagtgtatggagttgttcctaaaagcaagattcacggtatgtttaatgaagaccagtgtttttagcctgtgtcaatctatgcaaaatggaatcgagtattgatcaactgttaggagaatgagaccgatggaaacagccaattcaattactcagatattagaaaccaacttttccttcagtgggagagatgtcagaccattttatctttccttttatataatctatttttgcacagtctctattacacagttgtagaactggaccagatagttttgtgggcagtttttgcattattttagcctgacagtttttggttccatttcaggtgatcagaatcctcaaattgcggcacatgtcataagtgaggccagcagtaaaacaacatctggtaagtcacacagcatctgagcggtagccacccaaggggaaaggctgggatgccgaagtcatgttacctaatggttaaactcctcttttcccctgggacccaatttacaaacctacccctacacttctcctattcccttctttgtcttcaaagtgagttcaaatgcacagatgggacttagagggacaaaaggaggtggaatgcaatctggatgttctcattatgttcttgctcaatggctgattctaaatgatgaattactgggtggagggaccattgttctgacaacatagaagaaatggcatgtagtgacctcctgactgggagcatccctcctcctaacccatcttcactgtgtggaaatgggcctcatggggtatttcctgccatctgtcaatccctgtatgattaagctcagcctcactgaggccaacctcagggaaagtaaaggtaaaatcattctgtaaagatcaataggtcccaagacgttacattttccaatgaagtaacaacagacgacatattgtgatcttttcaactctgaacgattttatttccatatacgttctgccaccattctagcctttagatattttttcccaaatgtgcatcttgcgataactggtgccaaagaatatgtcgtatctgataaatggatggaaacatgcacgctaacataaagtctcccatcaacataaaggcaagagcgtcagaggagtctttgaaaaattctacagagtgctccggaatggagttctaagcagtgcatgtgtgtgtgcatatgtgtatgtgtgtgacagggagagaaagagagatggacagagagagaaaaaagacactgcttcatctctgaagtggcttgggcttctcagtaggcgtaacacatggacagttatcattatcatggatcatggtaccaaagtaagagcactgaatagggagtttttgaacactgggattcaaggaccatgaccactgcttgctgggtgaccttgagcaagaccctttacctatgcagcagttttctacttcacctactttacagggtggctttgagcatcaaatcagctaatgtggccgaaagtgatgctgtcgagtgctgtacaaccgtaaggtgacactacttagtttacttcaccatggcttagatgtcaaaagggtgacataaagcccctcactaataccagttagttacacaatatttaataattttgtcaagtaccccttctctcttctggatcagatgacaacaacagagaaatctcctagaagaatagcttcccactggtctttttttgcctgtatctaaacccttgatcttggatatatttcatagagctcagattctcccaaaaggcttgtaatggatatcagtcctacaatatcttacagtctgcatcacaataggtttccaggggatcagatgggaagacagtaacattccacccccaccccagtcccaaacctcttcttcctacctagccatgctgctaaaatcttgccctacatcccacagcaagtactaaaattaggtaaggacgtaccaaagtaaacttactgaactaaaagattgagaacctgccctttttttctcaataaaatggttcaaaagggcaaacattctaatgaagcattgtttctggagtggtctggagggcccggatctgtcaggcatttcaggatgcctccctattagtaaagggcgagtcttaccaggtgggatcttgtgccctgatagacctaagactatcgaataggaattattttttaaaaagctcaaggaagcaaacacatcagtactttcacttttcctcaaccctcacccccatcagtcagtctagctttctgtgggagctgagatttcaagtcgggtgcacacactactttgaacccactcaacatctcagccgagaaaatggcacactgttggtgggtactctggcttagccacaagaatactggtactttcaagttggtggcgcccactacaatgggagatcaaaacataccgtgaaatgagcacacagtttattttcatacttccttgcctaattttagtccttgctgggggaggcagatcaggtttgcaacagcatgatcaggtaggaagaaatggggtcttttctctgtgctgaggctgagctaggtagactgacaactctctgactttgtaaaattcaaggcaagcaaggtattcatggtaatattagcaaaaatttggtccgagtaatttggtatgtataatttatgatgtcaaattttgaaatcatttgtgccttcttaagttcaaggcaaattggctataagaactctaacgagagaaagaaactcactgtgatctcttactttatttaatcttcacaagtctctgaaatatgctccaatatgagccccgtgttgcagatgaggaactgaagctcatggagatttagagacttgcccaagcttaaatagagcctagattggaacatggctctgtctgactctgaagcccatggaaggggccttgagaatccatccctatacaaagccaatatccaacattaaactatattttttgtcagaatgtgaaccatgctctgcttcacctcaccacaaactttccctttctttgtaacagtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctgaacagtgtcaccttgcaggctgtggtggagctgacgctgggagtcttcataatacagcacagcggttaagcccaccccctgttaactgcctatttataaccctaggatcctccttatggagaactatttattatacactccaaggcatgtagaactgtaataagtgaattacaggtcacatgaaaccaaaacgggccctgctccataagagcttatatatctgaagcagcaaccccactgatgcagacatccagagagtcctatgaaaagacaaggccattatgcacaggttgaattctgagtaaacagcagataacttgccaagttcagttttgtttctttgcgtgcagtgtctttccatggataatgcatttgatttatcagtgaagatgcagaagggaaatggggagcctcagctcacattcagttatggttgactctgggttcctatggccttgttggagggggccaggctctagaacgtctaacacagtggagaaccgaaaccccccccccccccccgccaccctctcggacagttattcattctctttcaatctctctctctccatctctctctttcagtctctctctctcaacctctttcttccaatctctctttctcaatctctctgtttccctttgtcagtctcttccctcccccagtctctcttctcaatccccctttctaacacacacacacacacacacacacacacacacacacacacacacacacacacagagtcaggccgttgctagtcagttctcttctttccaccctgtccctatctctaccactatagatgagggtgaggagtagggagtgcagccctgagcctgcccactcctcattacgaaatgactgtatttaaaggaaatctattgtatctacctgcagtctccattgtttccagagtgaacttgtaattatcttgttatttattttttgaataataaagacctcttaacatta

SEQ ID NO: 14 - DNA, 7825 bp, Artificial Sequence
  AAV6(MND.GFP)
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagcggcctcggcctctgcataaataaaaaaaattagtcagccatgagcttggacgcgttgcttaacgtgtttcaaatttcttccatgcacatctttattagatcttcacagcaacctacaggataagcaagacaggtgcaagtgcctcctttgggtatgaggaaactgaggtctaaagagatgaagtgatttgcccaaggctcatagcaatttattggtagagcaaagactagaattcagatctcttaactgcagcctattttccctattctgaactgttacatcagcatcaacaattatctaatggattggaacagtgtacacaggcagcttagctacgtcaagtcacgatttttactttaacttcaattccagagtcttggcctgatttccctcaagaccctacttatctttgcctttgcaaaatttatttttcttgcattatctttccagctaaattttatttaataaccatcagcatgctttttttgctttatgccatgtagacttgacctgaaaacctgccaggctttcattgagtttagtgattaaagaagtaaagttctgagaagcaattagttgatgggacaccagtcataaaatcaatccaaacttttgttgacatgtgtttctttctccatataccaggttcccgcttcgtattagtaagattgaaattgaaataagtctattgctggtggatgaatttgtcactttccttgaaactggtgaacccaaaaagttagacagtgataggaaaatactgccattgtctgttaagaagtctatgacatttcaaggcaagaatgaatatatggaagaagaaacttgtttcttctttacttacaaaaaggaaagcctggaagtgaatgatatgggtataattaaaaaaaaaaaaaaaaacaaaaaacctttacgtaacgtttttgctgggagagaagactacgaagcacattttccaggaagtgtgggctgcaacgattgtgcgctcttaactaatcctgagtaaggtggccactttgacagtctgccaccttctctgccaactttaacacaggcggccgcgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacctccatagaagacaccgactctagaggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcaataaaagatctttattttcattagatctgtgtgttggttttttgtgtgggcccatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaaggtaagatgaaccacaagcctttattaactaaatttggggtccttactaattcataggttggttctacccaaatgatggatgatggtagaaaccaaatagaagaatggtcttgtggcataatgtttgttgcctagtcaatgaagtctcatattcttgtctctggttaggatcttgggatctggagtcagactgcctgggttcaaatcttggctctgcccataccatctctgttatcctggggcaagtgcctcagtttccacatctgagaaatggggatggtattggtgtccatttcatagattaagtgagtttagccttgtaaaaagcttaggagggggtctgatacatagtaagcactatgtacgcactagctataattatttgctaaagttctgctttaaaagtaagctatttttttatggagacagcttttttcttttaaatttccagctaggcaagaagagcgtcaatttgatctaaaatttcataatgcttcagattaacatagacatggataagtcccagaatttgcagtcttttagtaaaagtagcattttctgtgtaattcttcacaagcactgattgtagttgcaggatgctcagtctccctctgagatgttttacatttttaaatggttagacttgcaggaacaaaagagcagagtaacttagtaggctgttttgcattcttaggaaaagaaaaccatcaggacttattttgttttcatgtattttttcacttccactgaggagtataattggctggtgttgacaaaataccaatcatagatgtaaaggagaaagttgattagttttctggctgttcctaaaattctggatgcagtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 15 - DNA, 7250 bp, Artificial Sequence
  CRISPR AAV donor template, 1 kb homology arms, codon-optimized CD40L cDNA, a WPRE3 element, and a synthetic polyadenylation sequence
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagcggcctcggcctctgcataaataaaaaaaattagtcagccatgagcttggacgcgttgcttaacgtgtttcaaatttcttccatgcacatctttattagatcttcacagcaacctacaggataagcaagacaggtgcaagtgcctcctttgggtatgaggaaactgaggtctaaagagatgaagtgatttgcccaaggctcatagcaatttattggtagagcaaagactagaattcagatctcttaactgcagcctattttccctattctgaactgttacatcagcatcaacaattatctaatggattggaacagtgtacacaggcagcttagctacgtcaagtcacgatttttactttaacttcaattccagagtcttggcctgatttccctcaagaccctacttatctttgcctttgcaaaatttatttttcttgcattatctttccagctaaattttatttaataaccatcagcatgctttttttgctttatgccatgtagacttgacctgaaaacctgccaggctttcattgagtttagtgattaaagaagtaaagttctgagaagcaattagttgatgggacaccagtcataaaatcaatccaaacttttgttgacatgtgtttctttctccatataccaggttcccgcttcgtattagtaagattgaaattgaaataagtctattgctggtggatgaatttgtcactttccttgaaactggtgaacccaaaaagttagacagtgataggaaaatactgccattgtctgttaagaagtctatgacatttcaaggcaagaatgaatatatggaagaagaaacttgtttcttctttacttacaaaaaggaaagcctggaagtgaatgatatgggtataattaaaaaaaaaaaaaaaaacaaaaaacctttacgtaacgtttttgctgggagagaagactacgaagcacattttccaggaagtgtgggctgcaacgattgtgcgctcttaactaatcctgagtaaggtggccactttgacagtctgccaccttctctgccaactttaacacaggcggccgccatgattgagacttataatcagacctctcctcgcagcgcggcaacggggctgccgatctctatgaagatcttcatgtaccttctgactgtcttcctcattacacaaatgataggatctgccttgtttgcagtctacttgcaccgccgactggataaaatcgaggacgagcgaaatctgcacgaagacttcgtgtttatgaagaccattcagcggtgcaatacaggcgaacgatccctgagcctgttgaactgcgaagaaatcaagagtcaattcgaggggttcgtcaaggacatcatgctcaacaaggaagagactaaaaaggagaattcttttgagatgcagaagggggaccaaaacccccagattgccgcccacgtgatcagcgaagcctccagtaagacgaccagtgttctgcaatgggccgagaagggatattacacgatgtccaataacctggtcacacttgagaacggaaagcaactcactgttaagaggcagggcctgtattacatctacgcccaggtaacgttttgctcgaaccgcgaggcatccagccaggctccgtttatcgcttccctgtgtctcaaaagccctggcaggtttgaacgcattctccttagggccgctaatacgcacagctctgcgaagccctgcggtcaacagtcgatccatctgggtggcgttttcgagcttcagccgggagccagtgtcttcgtcaacgtgacagatccctcccaggtctcacatgggaccgggtttaccagcttcggactgctgaagttgtgagtcgacgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccattctagctttatttgtgaaatttgtgatgctattgctttatttgtaaccattataagctgcaataaacaagttaacaacaacaattgcattcattttatgtttcaggttcagggggagatgtgggaggttttttaaagcgggcccatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaaggtaagatgaaccacaagcctttattaactaaatttggggtccttactaattcataggttggttctacccaaatgatggatgatggtagaaaccaaatagaagaatggtcttgtggcataatgtttgttgcctagtcaatgaagtctcatattcttgtctctggttaggatcttgggatctggagtcagactgcctgggttcaaatcttggctctgcccataccatctctgttatcctggggcaagtgcctcagtttccacatctgagaaatggggatggtattggtgtccatttcatagattaagtgagtttagccttgtaaaaagcttaggagggggtctgatacatagtaagcactatgtacgcactagctataattatttgctaaagttctgctttaaaagtaagctatttttttatggagacagcttttttcttttaaatttccagctaggcaagaagagcgtcaatttgatctaaaatttcataatgcttcagattaacatagacatggataagtcccagaatttgcagtcttttagtaaaagtagcattttctgtgtaattcttcacaagcactgattgtagttgcaggatgctcagtctccctctgagatgttttacatttttaaatggttagacttgcaggaacaaaagagcagagtaacttagtaggctgttttgcattcttaggaaaagaaaaccatcaggacttattttgttttcatgtattttttcacttccactgaggagtataattggctggtgttgacaaaataccaatcatagatgtaaaggagaaagttgattagttttctggctgttcctaaaattctggatgcagtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 16 - DNA, 7411 bp, Artificial Sequence
  pAAV.TPL8.4
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagcggcctcggcctctgcataaataaaaaaaattagtcagccatgagcttggacgcgttgcttaacgtgtttcaaatttcttccatgcacatctttattagatcttcacagcaacctacaggataagcaagacaggtgcaagtgcctcctttgggtatgaggaaactgaggtctaaagagatgaagtgatttgcccaaggctcatagcaatttattggtagagcaaagactagaattcagatctcttaactgcagcctattttccctattctgaactgttacatcagcatcaacaattatctaatggattggaacagtgtacacaggcagcttagctacgtcaagtcacgatttttactttaacttcaattccagagtcttggcctgatttccctcaagaccctacttatctttgcctttgcaaaatttatttttcttgcattatctttccagctaaattttatttaataaccatcagcatgctttttttgctttatgccatgtagacttgacctgaaaacctgccaggctttcattgagtttagtgattaaagaagtaaagttctgagaagcaattagttgatgggacaccagtcataaaatcaatccaaacttttgttgacatgtgtttctttctccatataccaggttcccgcttcgtattagtaagattgaaattgaaataagtctattgctggtggatgaatttgtcactttccttgaaactggtgaacccaaaaagttagacagtgataggaaaatactgccattgtctgttaagaagtctatgacatttcaaggcaagaatgaatatatggaagaagaaacttgtttcttctttacttacaaaaaggaaagcctggaagtgaatgatatgggtataattaaaaaaaaaaaaaaaaacaaaaaacctttacgtaacgtttttgctgggagagaagactacgaagcacattttccaggaagtgtgggctgcaacgattgtgcgctcttaactaatcctgagtaaggtggccactttgacagtctgccaccttctctgccaactttaacacaggcggccgccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagggcccgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcaataaaagatctttattttcattagatctgtgtgttggttttttgtgtgggcccatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaaggtaagatgaaccacaagcctttattaactaaatttggggtccttactaattcataggttggttctacccaaatgatggatgatggtagaaaccaaatagaagaatggtcttgtggcataatgtttgttgcctagtcaatgaagtctcatattcttgtctctggttaggatcttgggatctggagtcagactgcctgggttcaaatcttggctctgcccataccatctctgttatcctggggcaagtgcctcagtttccacatctgagaaatggggatggtattggtgtccatttcatagattaagtgagtttagccttgtaaaaagcttaggagggggtctgatacatagtaagcactatgtacgcactagctataattatttgctaaagttctgctttaaaagtaagctatttttttatggagacagcttttttcttttaaatttccagctaggcaagaagagcgtcaatttgatctaaaatttcataatgcttcagattaacatagacatggataagtcccagaatttgcagtcttttagtaaaagtagcattttctgtgtaattcttcacaagcactgattgtagttgcaggatgctcagtctccctctgagatgttttacatttttaaatggttagacttgcaggaacaaaagagcagagtaacttagtaggctgttttgcattcttaggaaaagaaaaccatcaggacttattttgttttcatgtattttttcacttccactgaggagtataattggctggtgttgacaaaataccaatcatagatgtaaaggagaaagttgattagttttctggctgttcctaaaattctggatgcagtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatg

SEQ ID NO: 17 - DNA, 1142 bp, Artificial Sequence
  Sequence of control ActB amplicon for ddPCR
actctgcaggttctatttgctttttcccagatgagctctttttctggtgtttgtctctctgactaggtgtctaagacagtgttgtgggtgtaggtactaacactggctcgtgtgacaaggccatgaggctggtgtaaagcggccttggagtgtgtattaagtaggtgcacagtaggtctgaacagactccccatcccaagaccccagcacacttagccgtgttctttgcactttctgcatgtcccccgtctggcctggctgtccccagtggcttccccagtgtgacatggtgtatctctgccttacagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggtgagtggagactgtctcccggctctgcctgacatgagggttacccctcggggctgtgctgtggaagctaagtcctgccctcatttccctctcaggcatggagtcctgtggcatccacgaaactaccttcaactccatcatgaagtgtgacgtggacatccgcaaagacctgtacgccaacacagtgctgtctggcggcaccaccatgtaccctggcattgccgacaggatgcagaaggagatcactgccctggcacccagcacaatgaagatcaaggtgggtgtctttcctgcctgagctgacctgggcaggtcggctgtggggtcctgtggtgtgtggggagctgtcacatccagggtcctcactgcctgtccccttccctcctcagatcatt

SEQ ID NO: 18 - DNA, 999 bp, Artificial Sequence
  Upstream homology arm
tgcttaacgtgtttcaaatttcttccatgcacatctttattagatcttcacagcaacctacaggataagcaagacaggtgcaagtgcctcctttgggtatgaggaaactgaggtctaaagagatgaagtgatttgcccaaggctcatagcaatttattggtagagcaaagactagaattcagatctcttaactgcagcctattttccctattctgaactgttacatcagcatcaacaattatctaatggattggaacagtgtacacaggcagcttagctacgtcaagtcacgatttttactttaacttcaattccagagtcttggcctgatttccctcaagaccctacttatctttgcctttgcaaaatttatttttcttgcattatctttccagctaaattttatttaataaccatcagcatgctttttttgctttatgccatgtagacttgacctgaaaacctgccaggctttcattgagtttagtgattaaagaagtaaagttctgagaagcaattagttgatgggacaccagtcataaaatcaatccaaacttttgttgacatgtgtttctttctccatataccaggttcccgcttcgtattagtaagattgaaattgaaataagtctattgctggtggatgaatttgtcactttccttgaaactggtgaacccaaaaagttagacagtgataggaaaatactgccattgtctgttaagaagtctatgacatttcaaggcaagaatgaatatatggaagaagaaacttgtttcttctttacttacaaaaaggaaagcctggaagtgaatgatatgggtataattaaaaaaaaaaaaaaaaacaaaaaacctttacgtaacgtttttgctgggagagaagactacgaagcacattttccaggaagtgtgggctgcaacgattgtgcgctcttaactaatcctgagtaaggtggccactttgacagtctgccaccttctctgccaactttaacacag

SEQ ID NO: 19 - DNA, 1000 bp, Artificial Sequence
  Downstream homology arm
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaaggtaagatgaaccacaagcctttattaactaaatttggggtccttactaattcataggttggttctacccaaatgatggatgatggtagaaaccaaatagaagaatggtcttgtggcataatgtttgttgcctagtcaatgaagtctcatattcttgtctctggttaggatcttgggatctggagtcagactgcctgggttcaaatcttggctctgcccataccatctctgttatcctggggcaagtgcctcagtttccacatctgagaaatggggatggtattggtgtccatttcatagattaagtgagtttagccttgtaaaaagcttaggagggggtctgatacatagtaagcactatgtacgcactagctataattatttgctaaagttctgctttaaaagtaagctatttttttatggagacagcttttttcttttaaatttccagctaggcaagaagagcgtcaatttgatctaaaatttcataatgcttcagattaacatagacatggataagtcccagaatttgcagtcttttagtaaaagtagcattttctgtgtaattcttcacaagcactgattgtagttgcaggatgctcagtctccctctgagatgttttacatttttaaatggttagacttgcaggaacaaaagagcagagtaacttagtaggctgttttgcattcttaggaaaagaaaaccatcaggacttattttgttttcatgtattttttcacttccactgaggagtataattggctggtgttgacaaaataccaatcatagatgtaaaggagaaagttgattagttttctggctgttcctaaaattctggatgcag

SEQ ID NO: 20 - DNA, 12792 bp, Artificial Sequence
  pEVL200 vector
gcgtataatggactattgtgtgctgataaggagaacataagcgcagaacaatatgtatctattccggtgttgtgttcctttgttattctgctattatgttctcttatagtgtgacgaaagcagcataattaatcgtcacttgttctttgattgtgttacgatatccagagacttagaaacgggggaaccgggatgagcaaggtaaaaatcggtgagttgatcaacacgcttgtgaatgaggtagaggcaattgatgcctcagaccgcccacaaggcgacaaaacgaagagaattaaagccgcagccgcacggtataagaacgcgttatttaatgataaaagaaagttccgtgggaaaggattgcagaaaagaataaccgcgaatacttttaacgcctatatgagcagggcaagaaagcggtttgatgataaattacatcatagctttgataaaaatattaataaattatcggaaaagtatcctctttacagcgaagaattatcttcatggctttctatgcctacggctaatattcgccagcacatgtcatcgttacaatctaaattgaaagaaataatgccgcttgccgaagagttatcaaatgtaagaataggctctaaaggcagtgatgcaaaaatagcaagactaataaaaaaatatccagattggagttttgctcttagtgatttaaacagtgatgattggaaggagcgccgtgactatctttataagttattccaacaaggctctgcgttgttagaagaactacaccagctcaaggtcaaccatgaggttctgtaccatctgcagctaagccctgcggagcgtacatctatacagcaacgatgggccgatgttctgcgcgagaagaagcgtaatgttgtggttattgactacccaacatacatgcagtctatctatgatattttgaataatcctgcgactttatttagtttaaacactcgttctggaatggcacctttggcctttgctctggctgcggtatcagggcgaagaatgattgagataatgtttcagggtgaatttgccgtttcaggaaagtatacggttaatttctcagggcaagctaaaaaacgctctgaagataaaagcgtaaccagaacgatttatactttatgcgaagcaaaattattcgttgaattattaacagaattgcgttcttgctctgctgcatctgatttcgatgaggttgttaaaggatatggaaaggatgatacaaggtctgagaacggcaggataaatgctattttagcaaaagcatttaacccttgggttaaatcatttttcggcgatgaccgtcgtgtttataaagatagccgcgctatttacgctcgcatcgcttatgagatgttcttccgcgtcgatccacggtggaaaaacgtcgacgaggatgtgttcttcatggagattctcggacacgacgatgagaacacccagctgcactataagcagttcaagctggccaacttctccagaacctggcgacctgaagttggggatgaaaacaccaggctggtggctctgcagaaactggacgatgaaatgccaggctttgccagaggtgacgctggcgtccgtctccatgaaaccgttaagcagctggtggagcaggacccatcagcaaaaataaccaacagcactctccgggcctttaaatttagcccgacgatgattagccggtacctggagtttgccgctgatgcattggggcagttcgttggcgagaacgggcagtggcagctgaagatagagacacctgcaatcgtcctgcctgatgaagaatccgttgaaaccatcgacgaaccggatgatgagtcccaagacgacgagctggatgaagatgaaattgagctcgacgagggtggcggcgatgaaccaaccgaagaggaagggccagaagaacatcagccaactgctctaaaacccgtcttcaagcctgcaaaaaataacggggacggaacgtacaagatagagtttgaatacgatggaaagcattatgcctggtccggccccgccgatagccctatggccgcaatgcgatccgcatgggaaacgtactacagctaaaagaaaagccaccggtgttaatcggtggcttttttattgaggcctgtccctacccatcccctgcaagggacggaaggattaggcggaaactgcagctgcaactacggacatcgccgtcccgactgcagggacttccccgcgtaaagcggggcttaaattcgggctggccaaccctatttttctgcaatcgctggcgatgttagtttcgtggatagcgtttccagcttttcaatggccagctcaaaatgtgctggcagcaccttctccagttccgtatcaatatcggtgatcggcagctctccacaagacatactccggcgaccgccacgaactacatcgcgcagcagctcccgttcgtagacacgcatgttgcccagagccgtttctgcagccgttaatatccggcgcagctcggcgatgattgccgggagatcatccacggttattgggttcggtgatgggttcctgcaggcgcggcggagagccatccagacgccgctaacccatgcgttacggtactgaaaactttgtgctatgtcgtttatcaggccccgaagttcttctttctgccgccagtccagtggttcaccggcgttcttaggctcaggctcgacaaaagcatactcgccgtttttccggatagctggcagaacctcgttcgtcacccacttgcggaaccgccaggctgtcgtcccctgtttcaccgcgtcgcggcagcggaggattatggtgtagaggccagattccgataccacatttacttccctggccatccgatcaagtttttgtgcctcggttaaaccgagggtcaatttttcatcatgatccagcttacgcaatgcatcagaagggttggctatattcaatgcagcacagatatccagcgccacaaaccacgggtcaccaccgacaagaaccacccgtatagggtggctttcctgaaatgaaaagacggagagagccttcattgcgcctccccggatttcagctgctcagaaagggacagggagcagccgcgagcttcctgcgtgagttcgcgcgcgacctgcagaagttccgcagcttcctgcaaatacagcgtggcctcataactggagatagtgcggtgagcagagcccacaagcgcttcaacctgcagcaggcgttcctcaatcgtctccagcaggccctgggcgtttaactgaatctggttcatgcgatcacctcgctgaccgggatacgggctgacagaacgaggacaaaacggctggcgaactggcgacgagcttctcgctcggatgatgcagtggtggaaaggcggtggatatgggattttttgtccgtgcggacgacagctgcaaatttgaatttgaacatggtatgcattcctatcttgtatagggtgctaccaccagagttgagaatctctataggggtggtagcccagacagggttctcaacaccggtacaagaagaaaccggcccaaccgaagttggccccatctgagccaccataattcaggtatgcgcagatttaacacacaaaaaaacacgctggcgcgtgttgtgcgcttcttgtcattcggggttgagaggcccggctgcagattttgctgcagcggggtaactctaccgccaaagcagaacgcacgtcaataatttaggtggatattttaccccgtgaccagtcacgtgcacaggtgtttttatagtttgctttactgactgatcagaacctgatcagttattggagtccggtaatcttattgatgaccgcagccaccttagatgttgtctcaaaccccatacggccacgaatgagccactggaacggaatagtcagcaggtacagcggaacgaaccacaaacggttcagacgctgccagaacgtcgcatcacgacgttccatccattcggtattgtcgacgacctggtaagcgtattgtcctggcgtttttgctgcttccgagtagcaatcctcttcaccacaaagaaagttacttatctgcttccagttttcgaacccttcttctttgagccgcttttccagctcattcctccacaaaacaggcacccatcctctgcgataaatcatgattatttgtcctttaaataaggctgtagaactgcaaaatcgctctcgttcacatgctgtacgtagatgcgtagcaaattgccgttccatccctgtaatccaccttctttggaaagatcgtccttgacctcacgaagaactttatccaatagccctgcggcacaagaaattgcctgctctggatcagcaaattcatattgattaataggtgattgccacacaccaaaaacaggaatcatcttttcggctaaacgcctctcctgttctttcttaatctcaagttgtaagcggaccagctcaccatccatcattttttgtagatcatgcgccactattcacccccactggccatcagcaaataaagcttcatactcggacaccggcaggcggcttccacggattgaaaggtcaagccaaccacgtccagatgggtcagccttatccgattcttcccaccgttctgcagctgtagcaaccaggcattctaccgccttcatgtagtcttctgtacggaaccagccgtagttaatgccaccatcagtaactgcccaggccatctttttctcttcggcctcaatagcccggatgcggttatcgcacagctcgcgacagtacttcagctgttcgtaatccagttgcttcaggaactctggtgtcgacgtcatagtggcttcaccttataggcttttagaagcgccctggcttcgtctgtgtggtcttccatgctcttatcgctggcaatgcagcaataaactccctcactatctgagaacccgttcatccgaatgatcgtgaatggaagttcccggccagttttataatcgctatagcttgtcgcgtcgtggctgaccttgaccacataagggtcgtagccctccacgatgacaaggcattcccgttgttttcccattacccctccggttatatcgccacggcttgccgctggcttagaaacgctttcagcagccttatttcgcgtactgatagcaggtccataaattcggtcatgtacagcgaggcgaacgttctcgcgatgctggccactggccacaggcgtaccgcctccatttcggttgctggcaacgcgttctccgcccacgcctccggtaccgccaccgggatagcctccagtgcctggataattactgattgtggggcgtccggaacgtgctctgttttggatcgagggttaccatgtatatctatatttagatccaaatcgcgatccacttcgatggtggttttttccaccttacgtgcgtgaattgataaaccggcctcgcggcgcttctccacgatattcatgaggaactcgaccgagtccgggtcaatggaacgcatcgtggggcgtgcatcgccgtctctggcgcgtctggtcttactggatagccccatagactccaggatgcctatgcagaggtctgcaggcgctttcttcttgcctttctctgtgttgaagccgccgatgcgtaaaacgttgtttagcagatcgcgccgttccggcgtgagcaggttatctctggcgcgtttgagggcgtccatgtctgcttcaccttccagggtttttggatcgataccgcagtcgcggaagtactgctgcagcgtcgccgatttgagggtgtagaaaccacgcatgcctatctcaacagcaggggtcgatttcactcggtaatcggttatggccgggaatttagcctggaactctgcgtcggcctgttcccgcgtcatggccgtagtgacgaactgctgccatcttccggcaacgcgataagcgtaggtaaagtgaatcaacgcttcttcacggtcaaggcgacgggcggttatctcatccagctgcatggtttcaaacaggcgcacttttttcaggccgccgtcgaaatagaattttaacgccacctcgtcgacatccagctgcagctccttttcgatgtcccagcggaccagctgggcctgctcatccagggacagggtgcgtttttttatcaactcatcgtgttcggcctggtcaggagtatcgacactcaggtggcgctccataagctgctcaaagaccagttcacgggcttctttacgtaaatccttaccgatgctgtttgcaagcgcgtcggtggccataggcgcgacctgatagccatcatcatgcatgatgcaaatcatgttgctggcataatcatttctggccgatgcctcgagcgcggcggctttaattttgagctgcatgaatgaagagttagccacgccgagtgaaattcggtcaccgtcaaagacaacgtctgtcagcagcccggagtggccagccgtttcgagcaaggcctgcgcgtaggcgcgtttgattttttccggatcggtttcacgtttaccgcgaagcttgtcgaaaccgataatgtattcctgagctgtacggtcgcggcgcagcatctggatggcgtcgctggggaccacttcgccgcagaacatgccgaaatggcggtggaagtgtttctcctcaatcgatacacctgaagatatcgacgggctgtagatgaggccgtcatattttttcaccatcactttaggctggttggtgaaatcgtcgacttccttctcctgtttgtttttctggttaacgcagagaaactttttgtcagggaactgtagtctcagctgcatggtaacgtcttcggcgaacgtcgaactgtcggtggccagcatgattcgttcgccgcgttgcactgcagcgataacctcggtcatgatccgatttttctcggtataaaatacgcggataggcttgttggtttcgcggttgcgaacgtcgaccgggagttcaatcacgtgaatttgcagccaggcaggtaggcccagctcctcgcgtcgcttcatcgccagttcagccaggtcaacaagcagatcgttggcatcggcatccaccataatggcatgctcttcagtacgcgccagcgcgtcgataagcgtgttgaatacgcctaccgggttttccatcgcacgcccggccagaatggcacgcaggccctgtgttgcttcatcgaagccgaagaagtcatgctggcgcatcagcggttgccagcagcctttaagtatggagttgatgcaaatagtcagcttgttggcatatggcgccatttcctgatagccgggatcctgataatgcagaatgtcggctttcgcgcctttcccttcggtcatcatttcatgcaggccgcctatcagggatacgcggtgcgcgacggaaacgccacgcgtggactgcagcatcagtggacgcaggaggcctgtcgatttacccgaccccatcccggcgcggacaataacgatgccctgcagctgtgcggcgtatgtcatcacctcatcggtcatcctggaggtttcaaaccgtttgtaagtgatgtgtgacgggcgaaggttcgggttggtgatgcgttcactgaacgaacgtgatgtttgcgcggcacggcatttgcgattcaaccggcgcgtaatgtgatctttaacggtaccgttataaatttctgcgatacccatatcccgcagcgtgctgctgaaaaggcgcataagttctttcgggctgtttggtaccgggcatgtcagcatgccaatatcaacggcgcgaagcagttctttggcaaaagtgcgtctgttcagacgcgggagagtacgcagcttattcagcgtgatcgacaacagatcggttgcacggctcagatgatttctcgttaactggcgagcgacttccttcagccctctcaggctgtgcaggtcgttaaaatcgctgcattccagctcagggtcatcctcaaaagttgggtaaacacatttgacgccggaaaacttctccatgatgtcgaatccggtgcggaggcctgtgttgccttttccttcagctgaggatttgcggtcgttatcgagagcgcaagtgatttgcgcagccgggtacatgttcaccagctgctcgacaacgtgaatcatgttgttagcggaaaccgcaatgactaccgcgtcaaagcgttttttcgggtcgtttctggtcgccagccagatggatgccccggtggcgaaaccctctgcagtcgcaattttttgcgccccctgcaggtcgccaataacaaagcatgcaccgacgaaatcaccgttagtgatggcgctggtctggaacttgccaccattcagatcgatacgttgccagccaacaatccgcccgtcttttcttccgtccaggtgggacagaggtatcgccatgtaagttgttggtccacggctccatttcgcactgtcgtgactggtcacgcgacgtatatcacaagcgccaaatacgtcacgaattcccttttttaccgcataaggccaggagccatcttcagctggcgaatgttcccaggcgcgatggaaagccaaccatccaagcaggcgttcctgctccatctgattgttttttaaatcattaacgcgttgttgttcagctcggaggcggcgtgcttcagcctggcgctccatgcgtgcacgttcttcttccggctgagcgaccacggtcgcaccattccgttgctgttcacggcgatactccgaaaacaggaatgaaaagccactccaggagccagcgtcatgcgctttttcaacgaagttaacgaaaggataactgatgccatccttgctctgctcaaggcgtgaatagatttccacacggcctttaaggctcttctgcagagcttccggggaggaattattgtaggtggtatagcgctctacaccaccgcgcggattgagctgaatcttatcagcacacgcaggccagttgataccggccatcttcgccagctcagtcagctcatcacgtgccgcgtcaagcagtgaaaacggatcgctgccaaagcgctccgcgtagaattcttgtaaggtcattttttagcctttccatgcgaattagcattttttcgggttgaaaaaatccgcaggagcagccacaataaacgcactatctttctgaaggacgtatctgcgttatcgtggctacttcctgaaaaaggcccgagtttgccgactcggtttttttttcgtcttttttcggctgctacggtctggttcaaccccgacaaagtatagatcggattaaaccagaattatagtcagcaataaaccctgttattgtatcatctaccctcaaccatgaacgatttgatcgtaccgactacttggtgcacaaattgaagatcacttttatcatggataacccgttgagagttagcactatcaaggtagtaatgctgctcgtcataacgggctaatcgttgaattgtgatctcgccgttattatcacaaaccagtacatcctcacccggtacaagcgtaagtgaagaatcgaccaggataacgtctcccggctggtagtttcgctgaatctggttcccgaccgtcagtgcgtaaacggtgttccgttgactcacgaacggcaggaatcgctctgtgttggcaggttctccaggctgccagtctctatccggtcctgtctctgtcgtaccaataacaggaacgcggtctggatcagattcagtgccatacagtatccattgcacgggcttacgcaggcattttgccagcgatagcccgatctccagcgacggcatcacgtcgccacgttctaagttttggacgcccggaagagagattcctacagcttctgccacttgcttcagcgtcagtttcagctctaaacggcgtgctttcagtcgttcgcctcgtgttttcatacccttaatcataaatgatctctttatagctggctataatttttataaattatacctagctttaattttcacttattgattataataatccccatgaaacccgaagaacttgtgcgccatttcggcgatgtggaaaaagcagcggttggcgtgggcgtgacacccggcgcagtctatcaatggctgcaagctggggagattccacctctacgacaaagcgatatagaggtccgtaccgcgtacaaattaaagagtgatttcacctctcagcgcatgggtaaggaagggcataacaaggggatcctctagacgcagaaaggcccacccgaaggtgagccagtgtgattacatttgcggcctaactgtggccagtccagttacgctggagtcactagtgcggccgcgacaacttgtctagggcccaatggcccatacacttagtgatgcgcatcgtacggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaacgagaccttagggccattagacttgaagtcaagcggccgcttacaactggaccttgctggtacatagaactgattaactgaccatttaaatcataccaacatggtcaaataaaacgaaaggctcagtcgaaagactgggcctttcgttttaatctgatcggcacgtaagaggttccaactttcaccataatgaaataagatcactaccgggcgtatttttgagttatcgagattttcaggagctaaggaagctaaaatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctcgctcaggcgcaatcacgaatgaataacggtttggttggtgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctaacctaggtgacagaagtcaaaagcctccggtcggaggcttttgactttctgctagatctgtttcaatgcggtgaagggccaggcagctggggattatgtccagacccggccagcatgttggttttatcgcatattcagcgttgtcgcgtttacccaggtaaaatggaagcagtgtatcgtctgcgtgaatgtgcaaatcaggaacgtaaccgtggtacatagatgcagtcccttgcgggtcgttcccttcaacgagtaggacgcggtgcccttgcaaggctaaccattgcgcctggtgtactgcagatgaggttttataaacccctcccttgtgtgacataacggaaagtacaaccgggtttttatcgtcaggtctttggtttgggttaccaaacacactccgcatatggctaatttggtcaattgtgtagccagcgcgacgttctactcggcccctcatctcaaaatcaggagccggtagacgaccagctttttccgcgtctctgatagcctgcggtgttacgccgatcaggtctgcaacttctgttataccccagcggcgagtaatacgacgcgcttccgggctgtcatcgccgaactgtgcgatggcaatagcgcgcgtcatttcctgaccgcgattgatacagtctttcagcaaattaattaacgacatcctgtttcctctcaaacatgcccttatctttgtgtttttcatcatactttacgtttttaaagcaaagcaacataaaaaaagcaaagtgacttagaaaacgcaaagttaaggttcaaatcaattttttgatgcgctacagaagctatttagcttcatctaagcgcaacggtattacttacgttggtatatttaaaacctaacttaatgattttaaatgataataaatcataccaattgctatcaaaagttaagcgaacatgctgattttcacgctgtttatacactttgaggcatctctatctcttccgtctctatattgaaacacaatcaaagaacatcaatccatgtgacatcccccactatctaagaacaccataacagaacacaacataggaatgcaacattaatgtatcaataattcggaacatatgcactatatcatatctcaattacggaacatatcagcacacaattgcccattatacgc

SEQ ID NO: 21 - DNA, 15435 bp, Artificial Sequence
  pEVL.200 CD40LG TALEN F
gcgtataatggactattgtgtgctgataaggagaacataagcgcagaacaatatgtatctattccggtgttgtgttcctttgttattctgctattatgttctcttatagtgtgacgaaagcagcataattaatcgccacttgttctttgattgtgttacgatatccagagacttagaaacgggggaaccgggatgagcaaggtaaaaatcggtgagttgatcaacacgcttgtgaatgaggtagaggcaattgatgcctcagaccgcccacaaggcgacaaaacgaagagaattaaagccgcagccgcacggtataagaacgcgttatttaatgataaaagaaagttccgtgggaaaggattgcagaaaagaataaccgcgaatacttttaacgcctatatgagcagggcaagaaagcggtttgatgataaattacatcatagctttgataaaaatattaataaattatcggaaaagtatcctctttacagcgaagaattatcttcatggctttctatgcctacggctaatattcgccagcacatgtcatcgttacaatctaaattgaaagaaataatgccgcttgccgaagagttatcaaatgtaagaataggctctaaaggcagtgatgcaaaaatagcaagactaataaaaaaatatccagattggagttttgctcttagtgatttaaacagtgatgattggaaggagcgccgtgactatctttataagttattccaacaaggctctgcgttgttagaagaactacaccagctcaaggtcaaccatgaggttctgtaccatctgcagctaagccctgcggagcgtacatctatacagcaacgatgggccgatgttctgcgcgagaagaagcgtaatgttgtggttattgactacccaacatacatgcagtctatctatgatattttgaataatcctgcgactttatttagtttaaacactcgttctggaatggcacctttggcctttgctctggctgcggtatcagggcgaagaatgattgagataatgtttcagggtgaatttgccgtttcaggaaagtatacggttaatttctcagggcaagctaaaaaacgctctgaagataaaagcgtaaccagaacgatttatactttatgcgaagcaaaattattcgttgaattattaacagaattgcgttcttgctctgctgcatctgatttcgatgaggttgttaaaggatatggaaaggatgatacaaggtctgagaacggcaggataaatgctattttagcaaaagcatttaacccttgggttaaatcatttttcggcgatgaccgtcgtgtttataaagatagccgcgctatttacgctcgcatcgcttatgagatgttcttccgcgtcgatccacggtggaaaaacgtcgacgaggatgtgttcttcatggagattctcggacacgacgatgagaacacccagctgcactataagcagttcaagctggccaacttctccagaacctggcgacctgaagttggggatgaaaacaccaggctggtggctctgcagaaactggacgatgaaatgccaggctttgccagaggtgacgctggcgtccgtctccatgaaaccgttaagcagctggtggagcaggacccatcagcaaaaataaccaacagcactctccgggcctttaaatttagcccgacgatgattagccggtacctggagtttgccgctgatgcattggggcagttcgttggcgagaacgggcagtggcagctgaagatagagacacctgcaatcgtcctgcctgatgaagaatccgttgaaaccatcgacgaaccggatgatgagtcccaagacgacgagctggatgaagatgaaattgagctcgacgagggtggcggcgatgaaccaaccgaagaggaagggccagaagaacatcagccaactgctctaaaacccgtcttcaagcctgcaaaaaataacggggacggaacgtacaagatagagtttgaatacgatggaaagcattatgcctggtccggccccgccgatagccctatggccgcaatgcgatccgcatgggaaacgtactacagctaaaagaaaagccaccggtgttaatcggtggcttttttattgaggcctgtccctacccatcccctgcaagggacggaaggattaggcggaaactgcagctgcaactacggacatcgccgtcccgactgcagggacttccccgcgtaaagcggggcttaaattcgggctggccaaccctatttttctgcaatcgctggcgatgttagtttcgtggatagcgtttccagcttttcaatggccagctcaaaatgtgctggcagcaccttctccagttccgtatcaatatcggtgatcggcagctctccacaagacatactccggcgaccgccacgaactacatcgcgcagcagctcccgttcgtagacacgcatgttgcccagagccgtttctgcagccgttaatatccggcgcagctcggcgatgattgccgggagatcatccacggttattgggttcggtgatgggttcctgcaggcgcggcggagagccatccagacgccgctaacccatgcgttacggtactgaaaactttgtgctatgtcgtttatcaggccccgaagttcttctttctgccgccagtccagtggttcaccggcgttcttaggctcaggctcgacaaaagcatactcgccgtttttccggatagctggcagaacctcgttcgtcacccacttgcggaaccgccaggctgtcgtcccctgtttcaccgcgtcgcggcagcggaggattatggtgtagaggccagattccgataccacatttacttccctggccatccgatcaagtttttgtgcctcggttaaaccgagggtcaatttttcatcatgatccagcttacgcaatgcatcagaagggttggctatattcaatgcagcacagatatccagcgccacaaaccacgggtcaccaccgacaagaaccacccgtatagggtggctttcctgaaatgaaaagacggagagagccttcattgcgcctccccggatttcagctgctcagaaagggacagggagcagccgcgagcttcctgcgtgagttcgcgcgcgacctgcagaagttccgcagcttcctgcaaatacagcgtggcctcataactggagatagtgcggtgagcagagcccacaagcgcttcaacctgcagcaggcgttcctcaatcgtctccagcaggccctgggcgtttaactgaatctggttcatgcgatcacctcgctgaccgggatacgggctgacagaacgaggacaaaacggctggcgaactggcgacgagcttctcgctcggatgatgcagtggtggaaaggcggtggatatgggattttttgtccgtgcggacgacagctgcaaatttgaatttgaacatggtatgcattcctatcttgtatagggtgctaccaccagagttgagaatctctataggggtggtagcccagacagggttctcaacaccggtacaagaagaaaccggcccaaccgaagttggccccatctgagccaccataattcaggtatgcgcagatttaacacacaaaaaaacacgctggcgcgtgttgtgcgcttcttgtcattcggggttgagaggcccggctgcagattttgctgcagcggggtaactctaccgccaaagcagaacgcacgtcaataatttaggtggatattttaccccgtgaccagtcacgtgcacaggtgtttttatagtttgctttactgactgatcagaacctgatcagttattggagtccggtaatcttattgatgaccgcagccaccttagatgttgtctcaaaccccatacggccacgaatgagccactggaacggaatagtcagcaggtacagcggaacgaaccacaaacggttcagacgctgccagaacgtcgcatcacgacgttccatccattcggtattgtcgacgacctggtaagcgtattgtcctggcgtttttgctgcttccgagtagcaatcctcttcaccacaaagaaagttacttatctgcttccagttttcgaacccttcttctttgagccgcttttccagctcattcctccacaaaacaggcacccatcctctgcgataaatcatgattatttgtcctttaaataaggctgtagaactgcaaaatcgctctcgttcacatgctgtacgtagatgcgtagcaaattgccgttccatccctgtaatccaccttctttggaaagatcgtccttgacctcacgaagaactttatccaatagccctgcggcacaagaaattgcctgctctggatcagcaaattcatattgattaataggtgattgccacacaccaaaaacaggaatcatcttttcggctaaacgcctctcctgttctttcttaatctcaagttgtaagcggaccagctcaccatccatcattttttgtagatcatgcgccactattcacccccactggccatcagcaaataaagcttcatactcggacaccggcaggcggcttccacggattgaaaggtcaagccaaccacgtccagatgggtcagccttatccgattcttcccaccgttctgcagctgtagcaaccaggcattctaccgccttcatgtagtcttctgtacggaaccagccgtagttaatgccaccatcagtaactgcccaggccatctttttctcttcggcctcaatagcccggatgcggttatcgcacagctcgcgacagtacttcagctgttcgtaatccagttgcttcaggaactctggtgtcgacgtcatagtggcttcaccttataggcttttagaagcgccctggcttcgtctgtgtggtcttccatgctcttatcgctggcaatgcagcaataaactccctcactatctgagaacccgttcatccgaatgatcgtgaatggaagttcccggccagttttataatcgctatagcttgtcgcgtcgtggctgaccttgaccacataagggtcgtagccctccacgatgacaaggcattcccgttgttttcccattacccctccggttatatcgccacggcttgccgctggcttagaaacgctttcagcagccttatttcgcgtactgatagcaggtccataaattcggtcatgtacagcgaggcgaacgttctcgcgatgctggccactggccacaggcgtaccgcctccatttcggttgctggcaacgcgttctccgcccacgcctccggtaccgccaccgggatagcctccagtgcctggataattactgattgtggggcgtccggaacgtgctctgttttggatcgagggttaccatgtatatctatatttagatccaaatcgcgatccacttcgatggtggttttttccaccttacgtgcgtgaattgataaaccggcctcgcggcgcttctccacgatattcatgaggaactcgaccgagtccgggtcaatggaacgcatcgtggggcgtgcatcgccgtctctggcgcgtctggtcttactggatagccccatagactccaggatgcctatgcagaggtctgcaggcgctttcttcttgcctttctctgtgttgaagccgccgatgcgtaaaacgttgtttagcagatcgcgccgttccggcgtgagcaggttatctctggcgcgtttgagggcgtccatgtctgcttcaccttccagggtttttggatcgataccgcagtcgcggaagtactgctgcagcgtcgccgatttgagggtgtagaaaccacgcatgcctatctcaacagcaggggtcgatttcactcggtaatcggttatggccgggaatttagcctggaactctgcgtcggcctgttcccgcgtcatggccgtagtgacgaactgctgccatcttccggcaacgcgataagcgtaggtaaagtgaatcaacgcttcttcacggtcaaggcgacgggcggttatctcatccagctgcatggtttcaaacaggcgcacttttttcaggccgccgtcgaaatagaattttaacgccacctcgtcgacatccagctgcagctccttttcgatgtcccagcggaccagctgggcctgctcatccagggacagggtgcgtttttttatcaactcatcgtgttcggcctggtcaggagtatcgacactcaggtggcgctccataagctgctcaaagaccagttcacgggcttctttacgtaaatccttaccgatgctgtttgcaagcgcgtcggtggccataggcgcgacctgatagccatcatcatgcatgatgcaaatcatgttgctggcataatcatttctggccgatgcctcgagcgcggcggctttaattttgagctgcatgaatgaagagttagccacgccgagtgaaattcggtcaccgtcaaagacaacgtctgtcagcagcccggagtggccagccgtttcgagcaaggcctgcgcgtaggcgcgtttgattttttccggatcggtttcacgtttaccgcgaagcttgtcgaaaccgataatgtattcctgagctgtacggtcgcggcgcagcatctggatggcgtcgctggggaccacttcgccgcagaacatgccgaaatggcggtggaagtgtttctcctcaatcgatacacctgaagatatcgacgggctgtagatgaggccgtcatattttttcaccatcactttaggctggttggtgaaatcgtcgacttccttctcctgtttgtttttctggttaacgcagagaaactttttgtcagggaactgtagtctcagctgcatggtaacgtcttcggcgaacgtcgaactgtcggtggccagcatgattcgttcgccgcgttgcactgcagcgataacctcggtcatgatccgatttttctcggtataaaatacgcggataggcttgttggtttcgcggttgcgaacgtcgaccgggagttcaatcacgtgaatttgcagccaggcaggtaggcccagctcctcgcgtcgcttcatcgccagttcagccaggtcaacaagcagatcgttggcatcggcatccaccataatggcatgctcttcagtacgcgccagcgcgtcgataagcgtgttgaatacgcctaccgggttttccatcgcacgcccggccagaatggcacgcaggccctgtgttgcttcatcgaagccgaagaagtcatgctggcgcatcagcggttgccagcagcctttaagtatggagttgatgcaaatagtcagcttgttggcatatggcgccatttcctgatagccgggatcctgataatgcagaatgtcggctttcgcgcctttcccttcggtcatcatttcatgcaggccgcctatcagggatacgcggtgcgcgacggaaacgccacgcgtggactgcagcatcagtggacgcaggaggcctgtcgatttacccgaccccatcccggcgcggacaataacgatgccctgcagctgtgcggcgtatgtcatcacctcatcggtcatcctggaggtttcaaaccgtttgtaagtgatgtgtgacgggcgaaggttcgggttggtgatgcgttcactgaacgaacgtgatgtttgcgcggcacggcatttgcgattcaaccggcgcgtaatgtgatctttaacggtaccgttataaatttctgcgatacccatatcccgcagcgtgctgctgaaaaggcgcataagttctttcgggctgtttggtaccgggcatgtcagcatgccaatatcaacggcgcgaagcagttctttggcaaaagtgcgtctgttcagacgcgggagagtacgcagcttattcagcgtgatcgacaacagatcggttgcacggctcagatgatttctcgttaactggcgagcgacttccttcagccctctcaggctgtgcaggtcgttaaaatcgctgcattccagctcagggtcatcctcaaaagttgggtaaacacatttgacgccggaaaacttctccatgatgtcgaatccggtgcggaggcctgtgttgccttttccttcagctgaggatttgcggtcgttatcgagagcgcaagtgatttgcgcagccgggtacatgttcaccagctgctcgacaacgtgaatcatgttgttagcggaaaccgcaatgactaccgcgtcaaagcgttttttcgggtcgtttctggtcgccagccagatggatgccccggtggcgaaaccctctgcagtcgcaattttttgcgccccctgcaggtcgccaataacaaagcatgcaccgacgaaatcaccgttagtgatggcgctggtctggaacttgccaccattcagatcgatacgttgccagccaacaatccgcccgtcttttcttccgtccaggtgggacagaggtatcgccatgtaagttgttggtccacggctccatttcgcactgtcgtgactggtcacgcgacgtatatcacaagcgccaaatacgtcacgaattcccttttttaccgcataaggccaggagccatcttcagctggcgaatgttcccaggcgcgatggaaagccaaccatccaagcaggcgttcctgctccatctgattgttttttaaatcattaacgcgttgttgttcagctcggaggcggcgtgcttcagcctggcgctccatgcgtgcacgttcttcttccggctgagcgaccacggtcgcaccattccgttgctgttcacggcgatactccgaaaacaggaatgaaaagccactccaggagccagcgtcatgcgctttttcaacgaagttaacgaaaggataactgatgccatccttgctctgctcaaggcgtgaatagatttccacacggcctttaaggctcttctgcagagcttccggggaggaattattgtaggtggtatagcgctctacaccaccgcgcggattgagctgaatcttatcagcacacgcaggccagttgataccggccatcttcgccagctcagtcagctcatcacgtgccgcgtcaagcagtgaaaacggatcgctgccaaagcgctccgcgtagaattcttgtaaggtcattttttagcctttccatgcgaattagcattttttcgggttgaaaaaatccgcaggagcagccacaataaacgcactatctttctgaaggacgtatctgcgttatcgtggctacttcctgaaaaaggcccgagtttgccgactcggtttttttttcgtcttttttcggctgctacggtctggttcaaccccgacaaagtatagatcggattaaaccagaattatagtcagcaataaaccctgttattgtatcatctaccctcaaccatgaacgatttgatcgtaccgactacttggtgcacaaattgaagatcacttttatcatggataacccgttgagagttagcactatcaaggtagtaatgctgctcgtcataacgggctaatcgttgaattgtgatctcgccgttattatcacaaaccagtacatcctcacccggtacaagcgtaagtgaagaatcgaccaggataacgtctcccggctggtagtttcgctgaatctggttcccgaccgtcagtgcgtaaacggtgttccgttgactcacgaacggcaggaatcgctctgtgttggcaggttctccaggctgccagtctctatccggtcctgtctctgtcgtaccaataacaggaacgcggtctggatcagattcagtgccatacagtatccattgcacgggcttacgcaggcattttgccagcgatagcccgatctccagcgacggcatcacgtcgccacgttctaagttttggacgcccggaagagagattcctacagcttctgccacttgcttcagcgtcagtttcagctctaaacggcgtgctttcagtcgttcgcctcgtgttttcatacccttaatcataaatgatctctttatagctggctataatttttataaattatacctagctttaattttcacttattgattataataatccccatgaaacccgaagaacttgtgcgccatttcggcgatgtggaaaaagcagcggttggcgtgggcgtgacacccggcgcagtctatcaatggctgcaagctggggagattccacctctacgacaaagcgatatagaggtccgtaccgcgtacaaattaaagagtgatttcacctctcagcgcatgggtaaggaagggcataacaaggggatcctctagacgcagaaaggcccacccgaaggtgagccagtgtgattacatttgcggcctaactgtggccagtccagttacgctggagtcactagtgcggccgcgacaacttgtctagggcccaatggcccatacacttagtgtaatacgactcactatagggagagcggccgctttttcagcaagattaagccgccaccatggcgccgcggcctcctaagaagaagcggaaagtcgaattcgtggatctgcgaacactgggctatagccagcagcagcaggagaagatcaaacccaaggtgaggtccacagtcgcacagcaccatgaagccctggtgggccacgggttcactcacgctcatattgtcgcactgtctcagcatccagccgctctgggaaccgtggcagtcacataccagcacatcattactgccctgcccgaggctacccatgaagacatcgtgggagtcggcaaacagtggagcggcgcacgggccctggaggctctgctgaccgacgcaggggaactgagaggaccccctctgcagctggatacagggcagctggtgaagattgctaagaggggaggggtgacagcaatggaagccgtccacgcaagcaggaacgcactgacaggggcccccctgaacctgaccccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgactccggaccaagtggtggctatcgccagccacgatggcgcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttctatcgccagcaacggtggcggcaagcaagcgctcgaaagcattgtggcccagctgagccggcctgatccggcgttggccgcgttgaccaacgaccacctggtcgctctggcttgcctgggaggacgccctgctatggacgctgtgaagaaaggactgccccacgcacccgaactgattagacgggtgaaccggagaatcggcgagagaacatcccatagggtggcaatctctagaactcagctggtcaagagtgaactggaggaaaagaaatcagagctgcgccacaagctgaaatacgtgcctcatgagtatatcgaactgatcgagattgctcgcaattcaacccaggaccggatcctggaaatgaaagtgatggagttctttatgaaagtctacggatatcgggggaaacacctgggagggagcagaaagccagatggggccatctacacagtgggatcccccatcgactatggcgtgattgtcgatactaaagcctacagcggaggctataacctgcctatcggccaggctgacgagatgcagagatacgtggaggaaaaccagacccgcaataagcatattaaccccaatgaatggtggaaagtgtatcctagctccgtcacagagttcaagtttctgttcgtgagcggacactttaagggcaactacaaagcacagctgactaggctgaatcatatcaccaactgcaatggagccgtgctgtctgtcgaggaactgctgatcgggggagagatgattaaggctggcacactgactctggaggaagtgaggcgcaagttcaacaatggggaaatcaacttctaacctgcaggatgataagctagccccgggcgtacggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaacgagaccttagggccattagacttgaagtcaagcggccgcttacaactggaccttgctggtacatagaactgattaactgaccatttaaatcataccaacatggtcaaataaaacgaaaggctcagtcgaaagactgggcctttcgttttaatctgatcggcacgtaagaggttccaactttcaccataatgaaataagatcactaccgggcgtattttgagttatcgagattttcaggagctaaggaagctaaaatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctcgctcaggcgcaatcacgaatgaataacggtttggttggtgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctaacctaggtgacagaagtcaaaagcctccggtcggaggcttttgactttctgctagatctgtttcaatgcggtgaagggccaggcagctggggattatgtccagacccggccagcatgttggttttatcgcatattcagcgttgtcgcgtttacccaggtaaaatggaagcagtgtatcgtctgcgtgaatgtgcaaatcaggaacgtaaccgtggtacatagatgcagtcccttgcgggtcgttcccttcaacgagtaggacgcggtgcccttgcaaggctaaccattgcgcctggtgtactgcagatgaggttttataaacccctcccttgtgtgacataacggaaagtacaaccgggtttttatcgtcaggtctttggtttgggttaccaaacacactccgcatatggctaatttggtcaattgtgtagccagcgcgacgttctactcggcccctcatctcaaaatcaggagccggtagacgaccagctttttccgcgtctctgatagcctgcggtgttacgccgatcaggtctgcaacttctgttataccccagcggcgagtaatacgacgcgcttccgggctgtcatcgccgaactgtgcgatggcaatagcgcgcgtcatttcctgaccgcgattgatacagtctttcagcaaattaattaacgacatcctgtttcctctcaaacatgcccttatctttgtgtttttcatcatactttacgtttttaaagcaaagcaacataaaaaaagcaaagtgacttagaaaacgcaaagttaaggttcaaatcaattttttgatgcgctacagaagctatttagcttcatctaagcgcaacggtattacttacgttggtatatttaaaacctaacttaatgattttaaatgataataaatcataccaattgctatcaaaagttaagcgaacatgctgattttcacgctgtttatacactttgaggcatctctatctcttccgtctctatattgaaacacaatcaaagaacatcaatccatgtgacatcccccactatctaagaacaccataacagaacacaacataggaatgcaacattaatgtatcaataattcggaacatatgcactatatcatatctcaattacggaacatatcagcacacaattgcccattatacgc

SEQ ID NO: 22 - DNA, 15435 bp, Artificial Sequence
  pEVL200 .CD40LG TALEN R
gcgtataatggactattgtgtgctgataaggagaacataagcgcagaacaatatgtatctattccggtgttgtgttcctttgttattctgctattatgttctcttatagtgtgacgaaagcagcataattaatcgccacttgttctttgattgtgttacgatatccagagacttagaaacgggggaaccgggatgagcaaggtaaaaatcggtgagttgatcaacacgcttgtgaatgaggtagaggcaattgatgcctcagaccgcccacaaggcgacaaaacgaagagaattaaagccgcagccgcacggtataagaacgcgttatttaatgataaaagaaagttccgtgggaaaggattgcagaaaagaataaccgcgaatacttttaacgcctatatgagcagggcaagaaagcggtttgatgataaattacatcatagctttgataaaaatattaataaattatcggaaaagtatcctctttacagcgaagaattatcttcatggctttctatgcctacggctaatattcgccagcacatgtcatcgttacaatctaaattgaaagaaataatgccgcttgccgaagagttatcaaatgtaagaataggctctaaaggcagtgatgcaaaaatagcaagactaataaaaaaatatccagattggagttttgctcttagtgatttaaacagtgatgattggaaggagcgccgtgactatctttataagttattccaacaaggctctgcgttgttagaagaactacaccagctcaaggtcaaccatgaggttctgtaccatctgcagctaagccctgcggagcgtacatctatacagcaacgatgggccgatgttctgcgcgagaagaagcgtaatgttgtggttattgactacccaacatacatgcagtctatctatgatattttgaataatcctgcgactttatttagtttaaacactcgttctggaatggcacctttggcctttgctctggctgcggtatcagggcgaagaatgattgagataatgtttcagggtgaatttgccgtttcaggaaagtatacggttaatttctcagggcaagctaaaaaacgctctgaagataaaagcgtaaccagaacgatttatactttatgcgaagcaaaattattcgttgaattattaacagaattgcgttcttgctctgctgcatctgatttcgatgaggttgttaaaggatatggaaaggatgatacaaggtctgagaacggcaggataaatgctattttagcaaaagcatttaacccttgggttaaatcatttttcggcgatgaccgtcgtgtttataaagatagccgcgctatttacgctcgcatcgcttatgagatgttcttccgcgtcgatccacggtggaaaaacgtcgacgaggatgtgttcttcatggagattctcggacacgacgatgagaacacccagctgcactataagcagttcaagctggccaacttctccagaacctggcgacctgaagttggggatgaaaacaccaggctggtggctctgcagaaactggacgatgaaatgccaggctttgccagaggtgacgctggcgtccgtctccatgaaaccgttaagcagctggtggagcaggacccatcagcaaaaataaccaacagcactctccgggcctttaaatttagcccgacgatgattagccggtacctggagtttgccgctgatgcattggggcagttcgttggcgagaacgggcagtggcagctgaagatagagacacctgcaatcgtcctgcctgatgaagaatccgttgaaaccatcgacgaaccggatgatgagtcccaagacgacgagctggatgaagatgaaattgagctcgacgagggtggcggcgatgaaccaaccgaagaggaagggccagaagaacatcagccaactgctctaaaacccgtcttcaagcctgcaaaaaataacggggacggaacgtacaagatagagtttgaatacgatggaaagcattatgcctggtccggccccgccgatagccctatggccgcaatgcgatccgcatgggaaacgtactacagctaaaagaaaagccaccggtgttaatcggtggcttttttattgaggcctgtccctacccatcccctgcaagggacggaaggattaggcggaaactgcagctgcaactacggacatcgccgtcccgactgcagggacttccccgcgtaaagcggggcttaaattcgggctggccaaccctatttttctgcaatcgctggcgatgttagtttcgtggatagcgtttccagcttttcaatggccagctcaaaatgtgctggcagcaccttctccagttccgtatcaatatcggtgatcggcagctctccacaagacatactccggcgaccgccacgaactacatcgcgcagcagctcccgttcgtagacacgcatgttgcccagagccgtttctgcagccgttaatatccggcgcagctcggcgatgattgccgggagatcatccacggttattgggttcggtgatgggttcctgcaggcgcggcggagagccatccagacgccgctaacccatgcgttacggtactgaaaactttgtgctatgtcgtttatcaggccccgaagttcttctttctgccgccagtccagtggttcaccggcgttcttaggctcaggctcgacaaaagcatactcgccgtttttccggatagctggcagaacctcgttcgtcacccacttgcggaaccgccaggctgtcgtcccctgtttcaccgcgtcgcggcagcggaggattatggtgtagaggccagattccgataccacatttacttccctggccatccgatcaagtttttgtgcctcggttaaaccgagggtcaatttttcatcatgatccagcttacgcaatgcatcagaagggttggctatattcaatgcagcacagatatccagcgccacaaaccacgggtcaccaccgacaagaaccacccgtatagggtggctttcctgaaatgaaaagacggagagagccttcattgcgcctccccggatttcagctgctcagaaagggacagggagcagccgcgagcttcctgcgtgagttcgcgcgcgacctgcagaagttccgcagcttcctgcaaatacagcgtggcctcataactggagatagtgcggtgagcagagcccacaagcgcttcaacctgcagcaggcgttcctcaatcgtctccagcaggccctgggcgtttaactgaatctggttcatgcgatcacctcgctgaccgggatacgggctgacagaacgaggacaaaacggctggcgaactggcgacgagcttctcgctcggatgatgcagtggtggaaaggcggtggatatgggattttttgtccgtgcggacgacagctgcaaatttgaatttgaacatggtatgcattcctatcttgtatagggtgctaccaccagagttgagaatctctataggggtggtagcccagacagggttctcaacaccggtacaagaagaaaccggcccaaccgaagttggccccatctgagccaccataattcaggtatgcgcagatttaacacacaaaaaaacacgctggcgcgtgttgtgcgcttcttgtcattcggggttgagaggcccggctgcagattttgctgcagcggggtaactctaccgccaaagcagaacgcacgtcaataatttaggtggatattttaccccgtgaccagtcacgtgcacaggtgtttttatagtttgctttactgactgatcagaacctgatcagttattggagtccggtaatcttattgatgaccgcagccaccttagatgttgtctcaaaccccatacggccacgaatgagccactggaacggaatagtcagcaggtacagcggaacgaaccacaaacggttcagacgctgccagaacgtcgcatcacgacgttccatccattcggtattgtcgacgacctggtaagcgtattgtcctggcgtttttgctgcttccgagtagcaatcctcttcaccacaaagaaagttacttatctgcttccagttttcgaacccttcttctttgagccgcttttccagctcattcctccacaaaacaggcacccatcctctgcgataaatcatgattatttgtcctttaaataaggctgtagaactgcaaaatcgctctcgttcacatgctgtacgtagatgcgtagcaaattgccgttccatccctgtaatccaccttctttggaaagatcgtccttgacctcacgaagaactttatccaatagccctgcggcacaagaaattgcctgctctggatcagcaaattcatattgattaataggtgattgccacacaccaaaaacaggaatcatcttttcggctaaacgcctctcctgttctttcttaatctcaagttgtaagcggaccagctcaccatccatcattttttgtagatcatgcgccactattcacccccactggccatcagcaaataaagcttcatactcggacaccggcaggcggcttccacggattgaaaggtcaagccaaccacgtccagatgggtcagccttatccgattcttcccaccgttctgcagctgtagcaaccaggcattctaccgccttcatgtagtcttctgtacggaaccagccgtagttaatgccaccatcagtaactgcccaggccatctttttctcttcggcctcaatagcccggatgcggttatcgcacagctcgcgacagtacttcagctgttcgtaatccagttgcttcaggaactctggtgtcgacgtcatagtggcttcaccttataggcttttagaagcgccctggcttcgtctgtgtggtcttccatgctcttatcgctggcaatgcagcaataaactccctcactatctgagaacccgttcatccgaatgatcgtgaatggaagttcccggccagttttataatcgctatagcttgtcgcgtcgtggctgaccttgaccacataagggtcgtagccctccacgatgacaaggcattcccgttgttttcccattacccctccggttatatcgccacggcttgccgctggcttagaaacgctttcagcagccttatttcgcgtactgatagcaggtccataaattcggtcatgtacagcgaggcgaacgttctcgcgatgctggccactggccacaggcgtaccgcctccatttcggttgctggcaacgcgttctccgcccacgcctccggtaccgccaccgggatagcctccagtgcctggataattactgattgtggggcgtccggaacgtgctctgttttggatcgagggttaccatgtatatctatatttagatccaaatcgcgatccacttcgatggtggttttttccaccttacgtgcgtgaattgataaaccggcctcgcggcgcttctccacgatattcatgaggaactcgaccgagtccgggtcaatggaacgcatcgtggggcgtgcatcgccgtctctggcgcgtctggtcttactggatagccccatagactccaggatgcctatgcagaggtctgcaggcgctttcttcttgcctttctctgtgttgaagccgccgatgcgtaaaacgttgtttagcagatcgcgccgttccggcgtgagcaggttatctctggcgcgtttgagggcgtccatgtctgcttcaccttccagggtttttggatcgataccgcagtcgcggaagtactgctgcagcgtcgccgatttgagggtgtagaaaccacgcatgcctatctcaacagcaggggtcgatttcactcggtaatcggttatggccgggaatttagcctggaactctgcgtcggcctgttcccgcgtcatggccgtagtgacgaactgctgccatcttccggcaacgcgataagcgtaggtaaagtgaatcaacgcttcttcacggtcaaggcgacgggcggttatctcatccagctgcatggtttcaaacaggcgcacttttttcaggccgccgtcgaaatagaattttaacgccacctcgtcgacatccagctgcagctccttttcgatgtcccagcggaccagctgggcctgctcatccagggacagggtgcgtttttttatcaactcatcgtgttcggcctggtcaggagtatcgacactcaggtggcgctccataagctgctcaaagaccagttcacgggcttctttacgtaaatccttaccgatgctgtttgcaagcgcgtcggtggccataggcgcgacctgatagccatcatcatgcatgatgcaaatcatgttgctggcataatcatttctggccgatgcctcgagcgcggcggctttaattttgagctgcatgaatgaagagttagccacgccgagtgaaattcggtcaccgtcaaagacaacgtctgtcagcagcccggagtggccagccgtttcgagcaaggcctgcgcgtaggcgcgtttgattttttccggatcggtttcacgtttaccgcgaagcttgtcgaaaccgataatgtattcctgagctgtacggtcgcggcgcagcatctggatggcgtcgctggggaccacttcgccgcagaacatgccgaaatggcggtggaagtgtttctcctcaatcgatacacctgaagatatcgacgggctgtagatgaggccgtcatattttttcaccatcactttaggctggttggtgaaatcgtcgacttccttctcctgtttgtttttctggttaacgcagagaaactttttgtcagggaactgtagtctcagctgcatggtaacgtcttcggcgaacgtcgaactgtcggtggccagcatgattcgttcgccgcgttgcactgcagcgataacctcggtcatgatccgatttttctcggtataaaatacgcggataggcttgttggtttcgcggttgcgaacgtcgaccgggagttcaatcacgtgaatttgcagccaggcaggtaggcccagctcctcgcgtcgcttcatcgccagttcagccaggtcaacaagcagatcgttggcatcggcatccaccataatggcatgctcttcagtacgcgccagcgcgtcgataagcgtgttgaatacgcctaccgggttttccatcgcacgcccggccagaatggcacgcaggccctgtgttgcttcatcgaagccgaagaagtcatgctggcgcatcagcggttgccagcagcctttaagtatggagttgatgcaaatagtcagcttgttggcatatggcgccatttcctgatagccgggatcctgataatgcagaatgtcggctttcgcgcctttcccttcggtcatcatttcatgcaggccgcctatcagggatacgcggtgcgcgacggaaacgccacgcgtggactgcagcatcagtggacgcaggaggcctgtcgatttacccgaccccatcccggcgcggacaataacgatgccctgcagctgtgcggcgtatgtcatcacctcatcggtcatcctggaggtttcaaaccgtttgtaagtgatgtgtgacgggcgaaggttcgggttggtgatgcgttcactgaacgaacgtgatgtttgcgcggcacggcatttgcgattcaaccggcgcgtaatgtgatctttaacggtaccgttataaatttctgcgatacccatatcccgcagcgtgctgctgaaaaggcgcataagttctttcgggctgtttggtaccgggcatgtcagcatgccaatatcaacggcgcgaagcagttctttggcaaaagtgcgtctgttcagacgcgggagagtacgcagcttattcagcgtgatcgacaacagatcggttgcacggctcagatgatttctcgttaactggcgagcgacttccttcagccctctcaggctgtgcaggtcgttaaaatcgctgcattccagctcagggtcatcctcaaaagttgggtaaacacatttgacgccggaaaacttctccatgatgtcgaatccggtgcggaggcctgtgttgccttttccttcagctgaggatttgcggtcgttatcgagagcgcaagtgatttgcgcagccgggtacatgttcaccagctgctcgacaacgtgaatcatgttgttagcggaaaccgcaatgactaccgcgtcaaagcgttttttcgggtcgtttctggtcgccagccagatggatgccccggtggcgaaaccctctgcagtcgcaattttttgcgccccctgcaggtcgccaataacaaagcatgcaccgacgaaatcaccgttagtgatggcgctggtctggaacttgccaccattcagatcgatacgttgccagccaacaatccgcccgtcttttcttccgtccaggtgggacagaggtatcgccatgtaagttgttggtccacggctccatttcgcactgtcgtgactggtcacgcgacgtatatcacaagcgccaaatacgtcacgaattcccttttttaccgcataaggccaggagccatcttcagctggcgaatgttcccaggcgcgatggaaagccaaccatccaagcaggcgttcctgctccatctgattgttttttaaatcattaacgcgttgttgttcagctcggaggcggcgtgcttcagcctggcgctccatgcgtgcacgttcttcttccggctgagcgaccacggtcgcaccattccgttgctgttcacggcgatactccgaaaacaggaatgaaaagccactccaggagccagcgtcatgcgctttttcaacgaagttaacgaaaggataactgatgccatccttgctctgctcaaggcgtgaatagatttccacacggcctttaaggctcttctgcagagcttccggggaggaattattgtaggtggtatagcgctctacaccaccgcgcggattgagctgaatcttatcagcacacgcaggccagttgataccggccatcttcgccagctcagtcagctcatcacgtgccgcgtcaagcagtgaaaacggatcgctgccaaagcgctccgcgtagaattcttgtaaggtcattttttagcctttccatgcgaattagcattttttcgggttgaaaaaatccgcaggagcagccacaataaacgcactatctttctgaaggacgtatctgcgttatcgtggctacttcctgaaaaaggcccgagtttgccgactcggtttttttttcgtcttttttcggctgctacggtctggttcaaccccgacaaagtatagatcggattaaaccagaattatagtcagcaataaaccctgttattgtatcatctaccctcaaccatgaacgatttgatcgtaccgactacttggtgcacaaattgaagatcacttttatcatggataacccgttgagagttagcactatcaaggtagtaatgctgctcgtcataacgggctaatcgttgaattgtgatctcgccgttattatcacaaaccagtacatcctcacccggtacaagcgtaagtgaagaatcgaccaggataacgtctcccggctggtagtttcgctgaatctggttcccgaccgtcagtgcgtaaacggtgttccgttgactcacgaacggcaggaatcgctctgtgttggcaggttctccaggctgccagtctctatccggtcctgtctctgtcgtaccaataacaggaacgcggtctggatcagattcagtgccatacagtatccattgcacgggcttacgcaggcattttgccagcgatagcccgatctccagcgacggcatcacgtcgccacgttctaagttttggacgcccggaagagagattcctacagcttctgccacttgcttcagcgtcagtttcagctctaaacggcgtgctttcagtcgttcgcctcgtgttttcatacccttaatcataaatgatctctttatagctggctataatttttataaattatacctagctttaattttcacttattgattataataatccccatgaaacccgaagaacttgtgcgccatttcggcgatgtggaaaaagcagcggttggcgtgggcgtgacacccggcgcagtctatcaatggctgcaagctggggagattccacctctacgacaaagcgatatagaggtccgtaccgcgtacaaattaaagagtgatttcacctctcagcgcatgggtaaggaagggcataacaaggggatcctctagacgcagaaaggcccacccgaaggtgagccagtgtgattacatttgcggcctaactgtggccagtccagttacgctggagtcactagtgcggccgcgacaacttgtctagggcccaatggcccatacacttagtgtaatacgactcactatagggagagcggccgctttttcagcaagattaagccgccaccatggcgccgcggcctcctaagaagaagcggaaagtcgaattcgtggatctgcgaacactgggctatagccagcagcagcaggagaagatcaaacccaaggtgaggtccacagtcgcacagcaccatgaagccctggtgggccacgggttcactcacgctcatattgtcgcactgtctcagcatccagccgctctgggaaccgtggcagtcacataccagcacatcattactgccctgcccgaggctacccatgaagacatcgtgggagtcggcaaacagtggagcggcgcacgggccctggaggctctgctgaccgacgcaggggaactgagaggaccccctctgcagctggatacagggcagctggtgaagattgctaagaggggaggggtgacagcaatggaagccgtccacgcaagcaggaacgcactgacaggggcccccctgaacctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacaatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcgcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttctatcgccagcaacggtggcggcaagcaagcgctcgaaagcattgtggcccagctgagccggcctgatccggcgttggccgcgttgaccaacgaccacctggtcgctctggcttgcctgggaggacgccctgctatggacgctgtgaagaaaggactgccccacgcacccgaactgattagacgggtgaaccggagaatcggcgagagaacatcccatagggtggcaatctctagaactcagctggtcaagagtgaactggaggaaaagaaatcagagctgcgccacaagctgaaatacgtgcctcatgagtatatcgaactgatcgagattgctcgcaattcaacccaggaccggatcctggaaatgaaagtgatggagttctttatgaaagtctacggatatcgggggaaacacctgggagggagcagaaagccagatggggccatctacacagtgggatcccccatcgactatggcgtgattgtcgatactaaagcctacagcggaggctataacctgcctatcggccaggctgacgagatgcagagatacgtggaggaaaaccagacccgcaataagcatattaaccccaatgaatggtggaaagtgtatcctagctccgtcacagagttcaagtttctgttcgtgagcggacactttaagggcaactacaaagcacagctgactaggctgaatcatatcaccaactgcaatggagccgtgctgtctgtcgaggaactgctgatcgggggagagatgattaaggctggcacactgactctggaggaagtgaggcgcaagttcaacaatggggaaatcaacttctaacctgcaggatgataagctagccccgggcgtacggaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaacgagaccttagggccattagacttgaagtcaagcggccgcttacaactggaccttgctggtacatagaactgattaactgaccatttaaatcataccaacatggtcaaataaaacgaaaggctcagtcgaaagactgggcctttcgttttaatctgatcggcacgtaagaggttccaactttcaccataatgaaataagatcactaccgggcgtattttgagttatcgagattttcaggagctaaggaagctaaaatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctcgctcaggcgcaatcacgaatgaataacggtttggttggtgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctaacctaggtgacagaagtcaaaagcctccggtcggaggcttttgactttctgctagatctgtttcaatgcggtgaagggccaggcagctggggattatgtccagacccggccagcatgttggttttatcgcatattcagcgttgtcgcgtttacccaggtaaaatggaagcagtgtatcgtctgcgtgaatgtgcaaatcaggaacgtaaccgtggtacatagatgcagtcccttgcgggtcgttcccttcaacgagtaggacgcggtgcccttgcaaggctaaccattgcgcctggtgtactgcagatgaggttttataaacccctcccttgtgtgacataacggaaagtacaaccgggtttttatcgtcaggtctttggtttgggttaccaaacacactccgcatatggctaatttggtcaattgtgtagccagcgcgacgttctactcggcccctcatctcaaaatcaggagccggtagacgaccagctttttccgcgtctctgatagcctgcggtgttacgccgatcaggtctgcaacttctgttataccccagcggcgagtaatacgacgcgcttccgggctgtcatcgccgaactgtgcgatggcaatagcgcgcgtcatttcctgaccgcgattgatacagtctttcagcaaattaattaacgacatcctgtttcctctcaaacatgcccttatctttgtgtttttcatcatactttacgtttttaaagcaaagcaacataaaaaaagcaaagtgacttagaaaacgcaaagttaaggttcaaatcaattttttgatgcgctacagaagctatttagcttcatctaagcgcaacggtattacttacgttggtatatttaaaacctaacttaatgattttaaatgataataaatcataccaattgctatcaaaagttaagcgaacatgctgattttcacgctgtttatacactttgaggcatctctatctcttccgtctctatattgaaacacaatcaaagaacatcaatccatgtgacatcccccactatctaagaacaccataacagaacacaacataggaatgcaacattaatgtatcaataattcggaacatatgcactatatcatatctcaattacggaacatatcagcacacaattgcccattatacgc

SEQ ID NO: 23 - DNA, 15 bp, Artificial Sequence
  TALEN forward binding site
cttctcatgctgcct

SEQ ID NO: 24 - DNA, 15 bp, Artificial Sequence
  TALEN reverse binding site
agaagataccatttc

SEQ ID NO: 25 - DNA, 17 bp, Artificial Sequence
  TALEN cleavage site
ctgccaccttctctgcc

SEQ ID NO: 26 - DNA, 156 bp, Artificial Sequence
  CD40LG exon 1
atgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaag

SEQ ID NO: 27 - DNA, 132 bp, Artificial Sequence
  CD40LG exon 2
atagaagatgaaaggaatcttcatgaagattttgtattcatgaaaacgatacagagatgcaacacaggagaaagatccttatccttactgaactgtgaggagattaaaagccagtttgaaggctttgtgaag

SEQ ID NO: 28 - RNA, 20 nt, Artificial Sequence
  synthetic sgRNA
aaaguugaaaugguaucuuc

(19) United States
(12) Patent Application Publication
(10) Pub. No.: US 2021/0324381 A1
(43) Pub. Date: Oct. 21, 2021

(54) THERAPEUTIC GENOME EDITING IN X-LINKED HYPER IGM SYNDROME

(71) Applicant: Seattle Children's Hospital (dba Seattle Children's Research Institute), Seattle, WA (US)

(72) Inventors: David J. Rawlings, Seattle, WA (US); Daniel Thomson, Seattle, WA (US); Iram F. Khan, Seattle, WA (US)

(21) Appl. No.: 17/050,601

(22) PCT Filed: Apr. 24, 2019
(86) PCT No.: PCT/US19/28858

§ 371 Date: Oct. 26, 2020

Related U.S. Application Data
(60) Provisional application No. 62/663,485, filed on Apr. 27, 2018.

(51) Int. Cl. C12N 15/11; C12N 9/22; C12N 15/90; C07K 14/705; C12N 15/87; C12N 15/86; A61K 38/46; A61K 31/7088

(52) U.S. Cl. CPC C12N 15/11; C12N 9/22; C12N 15/907; C07K 14/70575; C12N 15/87; C12N 2750/14143; A61K 38/465; A61K 31/7088; C12N 2310/20; C12N 2800/80; C12N 15/86

(57) ABSTRACT

Described herein are compositions, systems, and methods for treating, inhibiting, or ameliorating X-linked hyper IgM syndrome (X-HIGM) in subjects that have been identified or selected as being ones that would benefit from a therapy to treat, inhibit, or ameliorate X-HIGM. The systems include nuclease and vector donor constructs configured for co-delivery to modify endogenous CD40LG locus.

### CROSS-REFERENCE TO RELATED APPLICATIONS

This application is a U.S. National Phase Application of PCT International Application Number PCT/US2019/028858, filed on Apr. 24, 2019, designating the United States of America and published in the English language, which is an International Application of and claims the benefit of priority to U.S. Provisional Application No. 62/663,485, filed on Apr. 27, 2018. The disclosures of the above-referenced applications are hereby expressly incorporated by reference in their entireties.

### REFERENCE TO SEQUENCE LISTING

The present application is being filed along with a Sequence Listing in electronic format. The Sequence Listing is provided as a file entitled SEQLISTSCRI192NP, created Mar. 9, 2021, which is approximately 102 Kb in size. The information in the electronic format of the Sequence Listing is incorporated herein by reference in its entirety.

### FIELD OF THE INVENTION

Aspects of the disclosure provided herein are generally related to endonuclease-based gene editing systems and methods. More particularly, alternatives herein relate to nucleic acids and vectors that are configured to provide efficient homology directed repair of genes and methods of repairing genetic deficiencies, such as X-linked hyper IgM syndrome.

### BACKGROUND

X-linked hyper IgM syndrome (X-HIGM) is a recessive primary immunodeficiency caused by an inactivating mutation in the CD40LG gene. Patients lack class-switched memory B cells and immunoglobulins G, A, and E (IgG, IgA, and IgE), making them susceptible to recurrent and opportunistic infections (Allen, R. C., et al., Science, 1993. 259(5097): p. 990-993; Aruffo, A., et al., Cell, 1993. 72(2): p. 291-300; Korthauer, U., et al., Nature, 1993. 361(6412): p. 539; Winkelstein, J. A., et al., Medicine, 2003. 82(6): p. 373-384). X-HIGM is currently treated with immunoglobulin replacement therapy or allogeneic bone marrow transplant. However, complications exist with both options and while bone marrow transplant is curative, many patients lack suitable donors (de la Morena, M. T., et al., J Allergy Clin Immunol, 2017. 139(4): p. 1282-1292).

Endonuclease-based systems have rapidly become significant gene editing tools in biomedical research, with their application for gene disruption and/or gene targeting demonstrated in a variety of cultured cell and model organism systems.

Endonuclease-based systems for gene editing allow scientists to edit genomes with unprecedented precision, efficiency, and flexibility. Examples of endonuclease-based approaches for gene editing include systems comprising, without limitations, zinc finger nucleases (ZFNs), TAL effector nucleases (TALENs), meganucleases (such as MegaTALs), and CRISPR/Cas9. The need for more approaches to inhibit and/or treat X-HIGM is manifest.

### SUMMARY

Some embodiments provided herein relate systems, methods, and compositions for therapeutic genome editing of X-linked hyper IgM syndrome. Some embodiments include a method for editing an CD40LG gene in a cell, comprising: (i) introducing a polynucleotide encoding a guide RNA (gRNA) into the cell, and (ii) introducing a template polynucleotide into the cell. In some embodiments, the gRNA comprises a nucleic acid having at least 95% identity to the nucleotide sequence of SEQ ID NO:12. In some embodiments, the gRNA comprises a nucleic acid having the nucleotide sequence of SEQ ID NO:12.

In some embodiments, introducing a polynucleotide encoding a gRNA into the cell comprises contacting the cell with a ribonucleoprotein (RNP) comprising a CAS9 protein and the polynucleotide encoding the gRNA. In some embodiments, the CAS9 protein and the polynucleotide encoding the gRNA have a ratio between 0.1:1 and 1:10. In some embodiments, the CAS9 protein and the polynucleotide encoding the gRNA have a ratio between 1:1 and 1:5. In some embodiments, the CAS9 protein and the polynucleotide encoding the gRNA have a ratio of about 1:1.2.

In some embodiments, the template polynucleotide encodes at least a portion of the CD40LG gene, or complement thereof. In some embodiments, the template polynucleotide encodes at least a portion of a wild-type CD40LG gene, or complement thereof. In some embodiments, the template polynucleotide comprises at least about 1 kb of the CD40LG gene. In some embodiments, the template polynucleotide comprises a nucleic acid having at least 95% identity to the nucleotide sequence of SEQ ID NO:15. In some embodiments, the template polynucleotide comprises the nucleotide sequence of SEQ ID NO:15.

In some embodiments, a viral vector comprises the template polynucleotide. In some embodiments, the vector is an adeno-associated viral (AAV) vector. In some embodiments, the vector is a self-complementary AAV (scAAV) vector.

In some embodiments, step (i) is performed before step (ii). In some embodiments, steps (i) and (ii) are performed simultaneously. In some embodiments, steps (i) and/or (ii) comprise performing nucleofection. In some embodiments, performing nucleofection comprises use of a LONZA system. In some embodiments, the system comprises use of a square wave pulse.

Some embodiments also include contacting the cell with IL-6. In some embodiments, the IL-6 has a concentration from about or at 20 ng/ml to 500 mg/ml. In some embodiments, the IL-6 has a concentration from about or at 50 ng/ml to 150 mg/ml. In some embodiments, the IL-6 has a concentration of about or at 100 mg/ml.

In some embodiments, the cell is incubated in a SFEMII medium.

In some embodiments, a population of cells comprises the cell, the population having a concentration from about or at 1×10⁵ cells/ml to 1×10⁶ cells/ml. In some embodiments, the population has a concentration from about or at 1×10⁵ cells/ml to 5×10⁵ cells/ml. In some embodiments, the population has a concentration from about or at 2.5×10⁵ cells/ml.

Some embodiments also include diluting the population of cells after steps (i) and (ii) are performed. In some embodiments, the population of cells is diluted about or at 16 hours after steps (i) and (ii) are performed. In some embodiments, the population of cells is diluted to about or at 250,000 cells/ml.

Some embodiments also include contacting the cell with stem cell factor (SCF), FMS-like tyrosine kinase-3 (Flt-3), thrombopoietin (TPO), a TPO receptor agonist, UM171, or stemregenin (SR1). In some embodiments, the TPO receptor agonist comprises Eltrombopag.

In some embodiments, steps (i) and/or (ii) comprise contacting the cell with an HDM2 protein. In some embodiments, the HDM2 protein has a concentration from about or at 1 nM to 50 nM. In some embodiments, the HDM2 protein has a concentration from about or at 6.25 nM to 25 nM.

In some embodiments, the cell is contacted with at least about or at 1000 MOI of the AAV. In some embodiments, the cell is contacted with at least about or at 2500 MOI of the AAV.

In some embodiments, the cell is contacted with at least about or at 100 μg/ml of the RNP. In some embodiments, the cell is contacted with at least about or at 200 μg/ml of the RNP.

In some embodiments, steps (i) and/or (ii) comprise contacting about or at 1,000,000 cells/20 μl nucleofection reaction, wherein the nucleofection reaction comprises the gRNA and/or the template polynucleotide. In some embodiments, the nucleofection reaction is performed in a volume of about or at 1 ml.

In some embodiments, the cell is mammalian. In some embodiments, the cell is human. In some embodiments, the cell is a primary cell. In some embodiments, the cell is a hematopoietic stem cell (HSC). In some embodiments, the cell is a T cell or a B cell. In some embodiments, the cell is a CD34+ cell. In some embodiments, the cell is ex vivo.

In some embodiments, the CD40LG gene has at least 95% identity with the nucleotide sequence of SEQ ID NO:13.

Some embodiments include a nucleic acid for homology directed repair (HDR) of CD40LG gene. In some embodiments, the nucleic acid comprises a first sequence encoding a CD40LG gene, a second sequence encoding one or more guide RNA cleavage sites, and a third sequence encoding one or more nuclease binding sites. In some embodiments, the CD40LG gene comprises the nucleic acid sequence set forth in SEQ ID NO: 13. In some embodiments, the second sequence comprises the nucleic acid sequence set forth in SEQ ID NO: 12. In some embodiments, the one or more nuclease binding sites comprises a forward and reverse transcription activator-like effector nuclease (TALEN) binding site. In some embodiments, the one or more nuclease binding sites is a clustered regularly interspaced short palindromic repeats (CRISPR) associated protein 9 (Cas9) binding site. In some embodiments, the nucleic acid further comprises one or more enhancer elements. In some embodiments, the nucleic acid further comprises homology arm sequences. In some embodiments, the nucleic acid further comprises a nucleic acid sequence encoding a promoter.

Some embodiments provided herein relate to a vector for promoting HDR of CD40L protein expression in a cell. In some embodiments, the vector comprises a first sequence encoding a CD40LG gene, a second sequence encoding one or more guide RNA cleavage sites, and a third sequence encoding one or more nuclease binding sites. In some embodiments, the CD40LG gene comprises the nucleic acid sequence set forth in SEQ ID NO: 13. In some embodiments, the second sequence comprises the nucleic acid sequence set forth in SEQ ID NO: 12. In some embodiments, the one or more nuclease binding sites comprises a forward and reverse transcription activator-like effector nuclease (TALEN) binding site. In some embodiments, the one or more nuclease binding sites is a clustered regularly interspaced short palindromic repeats (CRISPR) associated protein 9 (Cas9) binding site. In some embodiments, the vector further comprises one or more enhancer elements. In some embodiments, the vector is an adeno-associated viral vector (AAV). In some embodiments, the vector is a self-complementary AAV (scAAV). In some embodiments, the cell is a human cell. In some embodiments, the cell is a primary cell. In some embodiments, the cell is an autologous cell. In some embodiments, the cell is a T cell. In some embodiments, the cell is a hematopoietic stem cell (HSC). In some embodiments, the cell is a CD34⁺ HSC.

Some embodiments provided herein relate to a system for promoting HDR of CD40L protein expression in a cell. In some embodiments, the system comprises a vector as described herein and a nucleic acid encoding a nuclease. In some embodiments, the nuclease is a TALEN nuclease. In some embodiments, the nuclease is a Cas nuclease. In some embodiments, the vector and nucleic acid are configured for co-delivery to the cell. In some embodiments, co-delivery to the cell modifies endogenous CD40LG locus. In some embodiments, the cell is a primary human hematopoietic cell.

Some embodiments provided herein relate to a cell for expressing CD40L. In some embodiments, the cell comprises a nucleic acid, which comprises a first sequence encoding a CD40LG gene, a second sequence encoding a promoter, a third sequence encoding one or more guide RNA cleavage sites, and a fourth sequence encoding one or more nuclease binding sites. In some embodiments, the nucleic acid is in a vector. In some embodiments, the vector is an AAV. In some embodiments, the AAV is a scAAV. In some embodiments, the cell is a human cell. In some embodiments, the cell is a primary cell. In some embodiments, the cell is an autologous cell. In some embodiments, the cell is a T cell. In some embodiments, the cell is a HSC. In some embodiments, the cell is a CD34⁺ HSC.

Some embodiments provided herein relate to a method of promoting HDR of a CD40LG gene in a subject in need thereof. In some embodiments, the method comprises administering to a subject a cell as described herein or a vector as described herein and administering to the subject a nuclease. In some embodiments, the nuclease is a TALEN nuclease. In some embodiments, the nuclease is a Cas nuclease. In some embodiments, the nuclease is co-administered to the subject with the cell or with the vector. In some embodiments, the cell is from the subject and, wherein the cell is genetically modified by introducing a nucleic acid as described herein or a vector as described herein into the cell. In some embodiments, the administering is performed by adoptive cell transfer. In some embodiments, the cell is a human cell. In some embodiments, the cell is a primary cell. In some embodiments, the cell is an autologous cell. In some embodiments, the cell is a T cell. In some embodiments, the cell is a HSC. In some embodiments, the cell is a CD34⁺ HSC. In some embodiments, the subject is male. In some embodiments, the subject is suffering from X-linked hyper IgM (X-HIGM) syndrome.

Some embodiments provided herein relate to a method of treating, inhibiting, or ameliorating X-linked hyper IgM syndrome (X-HIGM) or disease symptoms associated with X-HIGM in a subject in need thereof. In some embodiments, the method comprises administering to a subject a cell as described herein or a vector as described herein and administering to the subject a nuclease. In some embodiments, the method further comprises identifying the subject as one that would benefit from receiving a therapy for X-HIGM or disease symptoms associated with X-HIGM and/or, optionally measuring an improvement in the progression of X-HIGM or an improvement in a disease symptom associated with X-HIGM in said subject. In some embodiments, the nuclease is a TALEN nuclease. In some embodiments, the nuclease is a CRISPR/Cas nuclease. In some embodiments, the nuclease is co-administered to the subject with the cell or with the vector. In some embodiments, the cell is from the subject, wherein the cell is genetically modified by introducing a nucleic acid as described herein or a vector as described herein into the cell. In some embodiments, the administering is performed by adoptive cell transfer. In some embodiments, the cell is a human cell. In some embodiments, the cell is a primary cell. In some embodiments, the cell is an autologous cell. In some embodiments, the cell is a T cell. In some embodiments, the cell is a HSC. In some embodiments, the cell is a CD34⁺ HSC. In some embodiments, the subject is male. In some embodiments, the method reduces bacterial or opportunistic infections. In some embodiments, the method reduces intermittent neutropenia.

### BRIEF DESCRIPTION OF THE DRAWINGS

FIG. 1 is a schematic illustrating that mutations in the CD40LG gene result in normal-elevated IgM, low IgG, and no IgE or IgA. X-linked hyper IgM (X-HIGM) patients suffer from bacterial/opportunistic infections and intermittent neutropenia.

FIG. 2 depicts a schematic illustrating human CD40LG locus showing ribonucleoprotein (RNP) recognition site relative to exons and translation start site, and also depicts AAV donor template with promoter-less CD40L cDNA and chimeric 3′UTR; shaded dashed lines show location of CD40LG homology. Talen and CRISPR nuclease sites and AAV6 donor template with 1 Kb CD40LG homology arms are also shown. The targeting construct contains deletions in the 5′ UTR that render it non-cleavable by either nuclease. The MND promoter allows tracking of editing events as the CD40LG locus is silent in hematopoietic stem cells (HSCs).

FIG. 3 is a schematic representation of an alternative of a method of CD34⁺ cell editing protocol. Cryopreserved CD34⁺ cells enriched from peripheral blood mononuclear cell (PBMC) mobilized adult donors were thawed and plated at 1×10⁶ cells/ml in serum-free stem cell growth media [CellGenix GMP SCGM medium (CellGenix Inc.) with thrombopoietin, stem cell factor, and FLT3 ligand (PeproTech) all at 100 ng/ml]. Either IL-3 (60 ng/ml) or IL-6 (100 ng/ml) was added to the media as noted. CD34⁺ cells were prestimulated in media for 48 hours at 37° C., then electroporated with the Neon Transfection System. Cells were dispensed into a 24 well plate containing 400 μL of media with donor template AAV at MOI between 1000 and 5000. Twenty-four hours after electroporation and AAV transduction, AAV containing media was removed and replaced with fresh stem cell growth media. Analysis of viability and GFP was performed at days 2 and 5 after editing.

FIG. 4A-and FIG. 4B depict results of efficient editing to introduce a GFP reporter at the CD40LG locus in CD34⁺ hematopoietic stem cells. FIG. 4A depicts representative flow cytometry plots for mock, AAV only of 1000 MOI, and AAV of 1000 MOI plus 50 μg/ml TALEN or 100 μg/ml RNP conditions showing gating for viability (left column) and GFP expression (right column) at 2 and 5 days post editing. FIG. 4B depicts cell viability and % GFP. The bar graphs depict forward and side scatter based viability 2 days post editing (left), and percentage of edited cells (% GFP⁺) as measured by flow cytometry 5 days post editing (right). Data are presented as mean±SEM. (N=9 replicates with 4 unique donors).

FIG. 5 depicts a bar graph showing confirmation of on-target integration of MND.GFP reporter (SEQ ID NO: 14). Bar graphs represent editing rates using MND.GFP reporter in CD34⁺ donors as determined by ddPCR or FACS. Cells grown in media containing IL-6, and treated with 2500 MOI of MND.GFP AAV6 and 200 μg/ml RNP. Data are presented as mean±SEM (N=2 replicates with 2 different donors).

FIG. 6A, FIG. 6B, FIG. 6C and FIG. 6D depict bar graphs showing results of methylcellulose colony forming unit assay on cells used to transplant NSG mice. FIG. 6A shows the total colony number, by type, counted 14 days after plating 500 mock or AAV+RNP treated cells in methylcellulose. FIG. 6B shows the total GFP⁺ colonies by colony type. FIG. 6C shows the percentage of GFP⁺ cells for each colony type. FIG. 6D shows the percentage of HDR-edited cells as determined by FACS.

FIG. 7A, FIG. 7B, FIG. 7C, FIG. 7D, FIG. 7E, FIG. 7F depict representative results comparing addition of IL-3 and IL-6 to culture media. Representative FACS plots depict cells grown in media containing either 60 ng/ml IL-3 (FIG. 7A) or 100 ng/ml IL-6 (FIG. 7B), 24 hours after treatment with AAV plus RNP. Top row, from left to right: cell viability measured by forward/side scatter, CD34⁺CD38⁻ staining, and CD133⁺CD90⁺ (LT-HSC) staining. Bottom row, from left to right: GFP expression among all viable cells, GFP expression among viable CD34⁺CD38⁻ cells, and GFP expression among CD34⁺CD38⁻ CD133⁺CD90⁺ cells. FIG. 7C depicts bar graphs that show cell viability measured by forward/side scatter 48 hours after editing. Cells receiving 1K MOI of AAV were electroporated with 100 ng/ml RNP, cells receiving 2.5K MOI of AAV were electroporated with 200 μg/ml RNP. Data are presented as mean±SEM. FIG. 7D depicts percentage of HDR-edited cells, measured by % GFP⁺5 days after editing. Data are presented as mean±SEM.

FIG. 7E depicts percentage of cells that stain CD34⁺CD38⁻CD133⁺CD90⁺, 48 hours after editing. Data presented as mean±SEM of fold change relative to mock, as different donors vary significantly in CD34⁺CD38⁻CD133⁺CD90⁺ staining. FIG. 7F depicts percentage of GFP⁺ within CD34⁺CD38⁻CD133⁺CD90⁺ cells, 48 hours after editing (N=3 donors, 3 replicates).

FIG. 8 depicts a schematic illustrating an AAV donor template containing identical 1 kb homology arms flanking human codon-optimized CD40L cDNA, a WPRE3 element and a synthetic polyadenylation sequence.

FIG. 9A and FIG. 9B depicts results of targeted integration of cDNA in edited cells. FIG. 9A depicts a bar graph showing cell viability as measured by flow cytometry (FSC/SSC live cell gate), 48 hours after editing for mock, AAV alone, or AAV+RNP treated cells. Data are presented as mean±SEM. FIG. 9B depicts editing rates in CD34⁺ donors as determined by ddPCR Data are presented as mean±SEM (N=2 donors, 3 replicates).

FIG. 10A and FIG. 10B depict bar graphs showing input transplant into NSG mice. FIG. 10A shows cell viability measured 2 days after editing (and 1 day after transplant) for the subset of cells maintained in vitro from NSG engraftment experiments. FIG. 10B shows that rate of HDR (% GFP⁺ measured at day 5) for cells from engraftment experiment maintained in vitro. All data shown represent mean±SEM.

FIG. 11 depicts an example gating strategy for cells harvested from NSG bone marrow and spleen. Lymphocyte and granulocyte populations were discriminated based on their size (forward scatter) and granularity (side scatter). These two major populations were then divided into separate lineages as defined by the expression of cell-type specific surface markers. GFP expression within each cell type is shown. For spleen samples, granulocyte population and CD34⁺/CD38⁻ populations were not analyzed.

FIG. 12A, FIG. 12B, FIG. 12C, FIG. 12D, FIG. 12E depict results of engraftment of edited cells in the bone marrow of NSG mice. FIG. 12A and FIG. 12B depict representative flow plots of cells harvested from the bone marrow of NSG mice 16 weeks following transplant using the gating strategy set forth in FIG. 11. FIG. 12A shows bone marrow harvested from mouse transplanted with untreated cells. FIG. 12B shows bone marrow harvested from mouse transplanted with cells treated with 2.5K MOI AAV+200 μg/ml RNP. Top row, from left to right: hCD45:mCD45 chimerism, Human CD45-gated CD33⁺, CD19⁺ and CD34⁺ staining. Bottom row, from left to right: GFP expression among hCD45⁺, CD33⁺, CD19⁺, and CD34⁺ cells. FIG. 12C shows total hCD45⁺ engraftment in bone marrow of NSG mice transplanted with mock, 1K MOI AAV+100 μg/ml RNP, or 2.5K MOI AAV+200 μg/ml RNP treated cells. Dots represent individual mice. Mean±SEM shown on graph. FIG. 12D shows percent HDR-edited cells (GFP⁺) of total hCD45⁺ population. Dots represent individual mice, mean±SEM shown on graph. FIG. 12E shows the ratio of CD33⁺ cells and CD19⁺ cells in total hCD45⁺ population. Data are presented as mean±SEM. Significance determined by two-way ANOVA.

FIG. 13A, FIG. 13B, FIG. 13C, FIG. 13D, FIG. 13E, FIG. 13F, FIG. 13G depicts results showing the impact of small molecules on engraftment of edited cells in NSG mice. FIG. 13A depicts bar graphs of cell viability 48 hours after editing determined by forward/side scatter. Data are presented as mean±SEM. FIG. 13B shows percentage of LT-HSCs 48 hours after editing, presented as mean±SEM. N=2 donors, 3 replicates. FIG. 13C depicts representative flow plots of cells harvested from the bone marrow of NSG mice 16 weeks following transplant of CD34⁺ cells grown in culture media containing UM171 and SR1 and treated with AAV plus RNP. FIG. 13D shows total hCD45⁺ engraftment in the bone marrow of NSG mice 12-16 weeks post transplantation with mock or edited (2.5K MOI AAV plus 200 μg/ml RNP treated) cells. Prior to transplant, cells were grown in media that contained various combinations of indicated small molecules. Each dot represents individual mouse, mean±SEM shown. FIG. 13E shows HDR-editing rate (% GFP⁺) among hCD45⁺ cells recovered from the bone marrow of NSG mice. Mean±SEM shown on graph. FIG. 13F shows percent CD33⁺ or CD19⁺ among total hCD45⁺ cells recovered from the bone marrow. Data are presented as mean±SEM. Significance determined by two-way ANOVA. FIG. 13G shows HDR-editing rate (% GFP⁺) among CD19⁺ cells, CD33⁺ cells, and CD34⁺ cells recovered from the bone marrow of NSG mice. Significance determined by paired T-test.

FIG. 14A, FIG. 14B, FIG. 14C, FIG. 14D depict results of engraftment of edited cells in the spleen of NSG mice. FIG. 14A and FIG. 14B show representative flow plots of cells harvested from the spleen of NSG mice 16 weeks following transplantation. Flow plots of FIG. 14A are from mouse transplanted with mock-treated cells. Top row, from left to right: hCD45:mCD45 chimerism, and human CD33⁺ and CD19⁺ staining. Bottom row plot shows GFP expression among, from left to right: hCD45⁺ cells, CD33⁺ cells, and CD19⁺ cells. Flow plots of FIG. 14B are from mouse transplanted with cells treated with 2.5K MOI AAV+200 μg/ml RNP. FIG. 14C shows total hCD45⁺ engraftment in spleen of NSG mice transplanted with mock, 1K MOI AAV+100 μg/ml RNP, or 2.5K MOI AAV+200 μg/ml RNP treated cells. Each dot represents individual mouse. Mean±SEM shown on graph. FIG. 14D shows percent HDR-edited cells among total hCD45⁺ population. Mean±SEM is shown on graph.

FIG. 15A, FIG. 15B, FIG. 15C, FIG. 15D, FIG. 15E show effects of small molecules UM171 and SR1 in vitro. FIG. 15A and FIG. 15B depicts representative flow plots from 48 hours after editing of LT-HSCs that show staining and GFP expression for cells grown without (FIG. 15A) or with (FIG. 15B) UM171 and SR1. Top row plots, from left: numbers on the plots denote viable cells defined by forward/side scatter, CD34⁺CD38⁻ population gated on all viable cells, and CD133⁺CD90+ cells within the CD34⁺/CD38⁻ population. Histograms depict GFP^{high} within live cells. Bottom row plots, from left: histograms denote GFP^{high} cells among all live cells, CD34⁺CD38⁻ and CD34⁺CD38⁻CD133⁺CD90⁺ cells. FIG. 15C shows GFP expression in the bulk population of indicated group 48 hours after editing, presented as mean±SEM. FIG. 15D shows GFP expression among cells staining as LT-HSCs 48 hours post editing. FIG. 15E shows the rate of HDR-editing as measured by GFP expression 5 days post editing. Data are presented as mean±SEM.

FIG. 16A, FIG. 16B, FIG. 16C, FIG. 16D. FIG. 16E, FIG. 16F, FIG. 16G show the impact of eltrombopag on engraftment of edited cells. FIG. 16A shows bar graphs that depict cell viability 48 hours after editing for mock or 1K MOI AAV+100 μg/ml RNP treated cells, grown with or without 3 μg/ml eltrombopag. Data shown represent mean±SEM. FIG. 16B shows the rate of HDR (% GFP⁺) as measured by FACS 5 days after editing. Data shown represent mean±SEM. FIG. 16C shows the percentage of LT-HSCs 48 hours after editing, presented as mean±SEM. FIG. 16D shows human CD45 engraftment in the bone marrow of NSG mice 12 weeks after transplant of cells. Each dot represents individual mouse, with mean shown. FIG. 16E shows the rate of HDR (% GFP⁺) among human cells harvested from the bone marrow of NSG mice. FIG. 16F shows hCD45⁺ engraftment in the spleen of NSG mice 12 weeks after the transplant of cells. FIG. 16G shows the rate of HDR (% GFP⁺) among human cells harvested from the spleen of NSG mice.

FIG. 17A and FIG. 17B depict the impact of time in culture after editing on engraftment of edited cells. CD34⁺ cells were transplanted into mice 1, 2 or 4 days post editing. FIG. 17A shows human CD45 engraftment in the bone marrow of NSG mice 12 weeks after transplant of cells. Each dot represents individual mouse, with mean shown. FIG. 17B shows the rate of HDR (% GFP⁺) among human cells harvested from the bone marrow of NSG mice.

FIG. 18 depicts a comparison of the effect of 48- and 72-hours pre-stimulation of CD34+ cells on the total human cells recovered from the bone marrow of NSGW41 recipient mice.

FIG. 19 depicts a comparison of the effect of 48- and 72-hours pre-stimulation of CD34+ cells on engraftment of edited (GFP+) cells in the bone marrow of W41 mice.

FIG. 20 depicts a comparison of the effect of 48- and 72-hours pre-stimulation of CD34+ cells on the total human cell engraftment in the spleens of W41 mice.

FIG. 21 depicts a comparison of the effect of 48- and 72-hours pre-stimulation of CD34+ cells on engraftment of edited (GFP+) cells in the spleens of NSGW41 mice.

FIG. 22 depicts a schematic showing the design of in vivo experimental studies established using Protocols A and B.

FIG. 23 depicts a comparison of percent hCD45+ cells recovered from the bone marrow of NSGW41 mice transplanted with CD34+ cells cultured using protocol A (dots) or B (squares).

FIG. 24 depicts a comparison of percent GFP+ among total hCD45+ cells recovered from the bone marrow of NSGW41 mice transplanted with CD34+ cells cultured using protocol A (dots) or B (squares).

FIG. 25 depicts a comparison of percent CD19+ cells among human CD45+ cells recovered from the bone marrow of NSGW41 mice engrafted CD34+ cells cultured using protocol A (dots) or B (squares).

FIG. 26 depicts a comparison of percent GFP+ cells among human CD19+ cells recovered from the bone marrow of NSGW41 mice transplanted with CD34+ cells cultured using protocol A (dots) or B (squares).

FIG. 27 depicts a comparison of percent CD33+ cells among human CD45+ cells recovered from the bone marrow of NSGW41 mice engrafted with CD34+ cells cultured using protocol A (dots) or B (squares).

FIG. 28 depicts a comparison of percent GFP+ cells among human CD33+ cells recovered from the bone marrow of NSGW41 mice engrafted with CD34+ cells cultured using protocol A (dots) or B (squares).

FIG. 29 depicts representative flow plots of cells harvested from the bone marrow of NSGW41 mice at 16 weeks following transplant. Upper panel shows bone marrow harvested from mouse transplanted with mock untreated cells: top row, from left to right: hCD45:mCD45 chimerism, human CD45-gated CD33+ and CD19+ staining; and bottom row, from left to right: GFP expression among hCD45+, CD33+ and CD19+ cells. Lower panel shows bone marrow harvested from mouse transplanted with cells treated with AAV plus RNP: top row, from left to right: hCD45:mCD45 chimerism, human CD45-gated CD33+ and CD19+ staining; and bottom row, from left to right: GFP expression among hCD45+, CD33+ and CD19+ cells. GFP+ human cells were present in all hematopoietic lineages consistent with sustained engraftment of long-term human HSC with HDR-editing of the CD40L locus.

FIG. 30 depicts a comparison of percent human CD45+ cells recovered from the spleens of NSGW41 mice transplanted with CD34+ cells cultured using protocol A (dots) or B (squares).

FIG. 31 depicts a comparison of percent GFP+ cells among human CD45+ cells recovered from the spleens of NSGW41 mice transplanted with CD34+ cells cultured using protocol A (dots) or B (squares).

FIG. 32 depicts a comparison of cell viability using various nucleofection programs on a LONZA system versus electroporation on a NEON system. Data from a single CD34+ donor is shown on the bar graph.

FIG. 33 depicts a comparison of percent HDR (GFP expression) using various nucleofection programs on a LONZA system versus electroporation on a NEON system. Data from a single CD34+ donor is shown on the bar graph.

FIG. 34 depicts a comparison of percent CD19+ cells among human CD45+ cells recovered from the spleens of NSGW41 mice transplanted with CD34+ cells cultured using protocol A (dots) or B (squares).

FIG. 35 depicts a comparison of percent GFP+ cells among human CD19+ cells recovered from the spleens of NSGW41 mice transplanted with CD34+ cells cultured using protocol A (dots) or B (squares).

FIG. 36 depicts a comparison of percent CD33+ cells among human CD45+ cells recovered from the spleens of NSGW41 mice transplanted with CD34+ cells cultured using protocol A (dots) or B (squares).

FIG. 37 depicts a comparison of percent GFP+ cells among human CD33+ cells recovered from the spleens of NSGW41 mice transplanted with CD34+ cells cultured using protocol A (dots) or B (squares).

FIG. 38 depicts representative flow plots of cells harvested from the spleens of NSGW41 mice 16 weeks following transplant. Upper panel shows spleen harvested from mouse transplanted with mock untreated cells: top row, from left to right: hCD45:mCD45 chimerism, human CD45-gated CD33+ and CD19+ staining; bottom row, from left to right: GFP expression among hCD45+, CD33+ and CD19+ cells. Lower panel shows spleen harvested from mouse transplanted with cells treated with AAV plus RNP: top row, from left to right: hCD45:mCD45 chimerism, human CD45-gated CD33+ and CD19+ staining; and bottom row, from left to right: GFP expression among hCD45+, CD33+ and CD19+ cells. GFP+ human cells were present in all hematopoietic lineages consistent with derivation of these cells from human HSC with HDR-editing of the CD40L locus.

FIG. 39: depicts a comparison of percent CD34+CD38low cells among human CD45+ cells recovered from the bone marrow of NSGW41 mice transplanted with CD34+ cells cultured using protocol A (dots) or B (squares).

FIG. 40 depicts a comparison of percent GFP+ cells among human CD34+CD38low CD45+ cells recovered from the bone marrow of NSGW41 mice transplanted with CD34+ cells cultured using protocol A (dots) or B (squares).

FIG. 41 depicts representative flow cytometry analysis of CD34+ gated on total human CD45+ cells from NSGW41 mice transplanted with mock or edited cells.

FIG. 42 depicts improvement in HDR rates by nucleofection of recombinant HDM2.

### DETAILED DESCRIPTION

In the following detailed description, reference is made to the accompanying drawings, which form a part hereof. In the drawings, similar symbols typically identify similar components, unless context dictates otherwise. The illustrative alternatives described in the detailed description, drawings, and claims are not meant to be limiting. Other alternatives may be utilized, and other changes may be made, without departing from the spirit or scope of the subject matter presented herein. It will be readily understood that the aspects of the present disclosure, as generally described herein, and illustrated in the figure, can be arranged, substituted, combined, separated, and designed in a wide variety of different configurations, all of which are explicitly contemplated herein.

### Definitions

In the description that follows, a number of terms are used extensively. The following definitions are provided to facilitate understanding of the present alternatives. Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Springs Harbor Press (Cold Springs Harbor, N Y 1989). For purposes of the present disclosure, the following terms are defined below.

The articles “a” and “an” are used herein to refer to one or to more than one (for example, to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight, or length.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least.” When used in the context of a process, the term “comprising” means that the process comprises at least the recited steps, but may include additional steps. When used in the context of a compound, composition or device, the term “comprising” means that the compound, composition or device comprises at least the recited features or components, but may also include additional features or components.

As used herein, a “subject” or a “patient” as described herein, have their plain and ordinary meaning when read in light of the specification, and may include but is not limited to, for example, an animal that is the object of treatment, observation or experiment. “Animal” comprises cold- and warm-blooded vertebrates and invertebrates such as fish, shellfish, reptiles and, in particular, mammals. “Mammal” comprises, without limitation, mice, rats, rabbits, guinea pigs, dogs, cats, sheep, goats, cows, horses, primates, such as monkeys, chimpanzees, and apes, and, in particular, humans. In some alternatives, the subject is human.

Some alternatives disclosed herein relate to selecting a subject or patient in need. In some alternatives, a patient is selected who is in need of treatment, amelioration, inhibition, progression, or improvement in disease symptoms or who is in need of curative therapy. In some alternatives, a patient is selected who has symptoms of X-linked hyper IgM syndrome (X-HIGM), who has been diagnosed with X-HIGM, or who is suspected of having X-HIGM. Such identification or selection of said subjects or patients in need can be made through clinical and/or diagnostic evaluation.

X-HIGM refers to a primary immune deficiency disorder characterized by defective CD40 signaling as a result of mutations in the CD40LG gene. The cell surface molecule CD40 is a member of the tumor necrosis factor receptor superfamily and is broadly expressed by immune, hematopoietic, vascular, epithelial, and other cells, including a wide range of tumor cells. CD40 itself lacks intrinsic kinase or other signal transduction activity, but rather mediates its diverse effects via an intricate series of downstream adapter molecules that differentially alter gene expression depending on cell type and microenvironment. As a potential target for novel cancer therapy, CD40 may mediate tumor regression through both an indirect effect of immune activation and a direct cytotoxic effect on the CD40-expressing tumor.

CD40 is known as a critical regulator of cellular and humoral immunity via its expression on B lymphocytes, dendritic cells, and monocytes (Grewal and Flavell, Annu Rev Immunol., 16:111 (1998); van Kooten and Banchereau, J Leukoc Biol., 67:2 (2000)).

CD40 is also expressed on the cell surface of many other non-immune cells, including endothelial cells, fibroblasts, hematopoietic progenitors, platelets and basal epithelial cells (Grewal and Flavell, Annu Rev Immunol., 16:111 (1998); van Kooten and Banchereau, J Leukoc Biol., 67:2 (2000); Young et al., Immunol Today, 9:502 (1998); Quezada et al., Annu Rev Immunol., 22:307 (2004). The CD40 ligand (CD40L), also known as CD154, is the chief ligand described for CD40 and is expressed primarily by activated T lymphocytes and platelets (van Kooten and Banchereau, J Leukoc Biol., 67:2 (2000); Armitage et al., Nature, 357:80 (1992)). Atherosclerosis, graft rejection, coagulation, infection control, and autoimmunity are all regulated by CD40-CD40L interactions (Grewal and Flavell, Annu Rev Immunol., 16:111 (1998); van Kooten and Banchereau, J Leukoc Biol., 67:2 (2000)).

The term “costimulatory molecule” encompasses any single molecule or combination of molecules which, when acting together with a MHC/peptide complex bound by a T cell antigen receptor (TCR) on the surface of a T cell, provides a co-stimulatory effect which achieves activation of the I cell that binds the peptide.

As used herein, the term “treatment” has its plain and ordinary meaning when read in light of the specification, and may include but is not limited to, for example, an intervention made in response to a disease, disorder or physiological condition manifested by a subject, particularly a subject suffering from X-HIGM. The aim of treatment may include, but is not limited to, one or more of the alleviation or prevention of symptoms, slowing or stopping the progression or worsening of a disease, disorder, or condition, curative treatment of the disease, disorder, or condition, and the remission of the disease, disorder, or condition. In some alternatives, “treatment” refers to both treatment of the underlying disease or treatment of the disease symptoms. For example, in some alternatives, treatments reduce, alleviate, ameliorate, or eradicate the symptom(s) of the disease and/or provide curative therapy of the disease.

“Adoptive cellular therapy” or “adoptive cell transfer,” as described herein, have their plain and ordinary meaning when read in light of the specification, and may include but is not limited to, for example, transfer of cells, most commonly immune-derived cells, back into the same patient or into a new recipient host with the goal of transferring the immunologic functionality and characteristics into the new host. In some alternatives, adoptive cellular therapy or adoptive cell transfer comprises administering cells for promoting homology directed repair of a CD40LG gene in a subject.

As used herein, “homology-directed repair” (HDR) has its plain and ordinary meaning when read in light of the specification, and may include but is not limited to, for example, DNA repair that takes place in cells, for example, during repair of a double-stranded break (DSB) in DNA. HDR requires nucleotide sequence homology and uses a donor polynucleotide to repair the sequence where the DSB (e.g., within a target DNA sequence) occurred. The donor polynucleotide generally has the requisite sequence homology with the sequence flanking the DSB so that the donor polynucleotide can serve as a suitable template for repair. HDR results in the transfer of genetic information from, for example, the donor polynucleotide to the DNA target sequence. HDR may result in alteration of the DNA target sequence (e.g., insertion, deletion, mutation) if the donor polynucleotide sequence differs from the DNA target sequence and part or all of the donor polynucleotide is incorporated into the DNA target sequence. In some alternatives, an entire donor polynucleotide, a portion of the donor polynucleotide, or a copy of the donor polynucleotide is integrated at the site of the DNA target sequence.

As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars or carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded. Nucleic acid sequences may be described herein with a SEQ ID NO, and are described throughout the application and included in Appendix I. In some alternatives, a nucleotide sequence described herein is 85%, 86%, 87%, 88%, 89%, 90%, 91%, 92%, 93%, 94%, 95%, 96%, 97%, 98%, 99% or 100% identical, or is within a range defined by any two of the aforementioned percentages, to a sequence of any one of SEQ ID NOs: 1-9 or 12-27.

As used herein, the term “fusion” or “fused” have their plain and ordinary meaning when read in light of the specification, and may include but is not limited to, for example, a first nucleic acid linked to a second nucleic acid by a phosphodiester bond, so that a coding sequence at the 3′ end of the first nucleic acid is in frame with a coding sequence at the 5′ end of the second nucleic acid, and by extension can further refer to a first polypeptide linked by a peptide bond to a second polypeptide at the C-terminus of the first polypeptide. As such, a “fused” (or “fusion of a”) nucleic acid or peptide as used herein refers to a configuration of molecules, and does not necessarily involve performing the act of joining two molecules together. By way of example, the fusion of a first nucleic acid to a second nucleic acid can encode a single polypeptide in which a first polypeptide sequence (encoded by the first nucleic acid) is fused to a second polypeptide sequence (encoded by the second nucleic acid). In some alternatives, the molecule comprising the fused nucleic acids is referred to as a fusion nucleic acid.

As used herein, the term “variant” has its plain and ordinary meaning when read in light of the specification, and may include but is not limited to, for example, a polynucleotide (or polypeptide) having a sequence substantially similar to a reference polynucleotide (or polypeptide). In the case of a polynucleotide, a variant can have deletions, substitutions, or additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least 50%, 55%, 60%, 65%, 70%, 75%, 80%, 85%, 90%, 91%, 92%, 93%, 94%, 95%, 96%, 97%, 98%, or 99% or more sequence identity to the reference polynucleotide as determined by sequence alignment programs known by skilled artisans, or an amount within a range defined by any two of the aforementioned values. In the case of a polypeptide, a variant can have deletions, substitutions, or additions of one or more amino acids in comparison to the reference polypeptide. Similarities and/or differences in sequences between a variant and the reference polypeptide can be detected using conventional techniques known in the art, for example Western blot. Generally, a variant of a polypeptide, can have at least 60%, 65%, 70%, 75%, 80%, 85%, 90%, 91%, 92%, 93%, 94%, 95%, 96%, 97%, 98%, or 99% or more sequence identity to the reference polypeptide as determined by sequence alignment programs known by skilled artisans, or an amount within a range defined by any two of the aforementioned values.

A “regulatory element” is a nucleotide sequence that modulates the activity of a core promoter. For example, a regulatory element may contain a nucleotide sequence that binds with cellular factors enabling transcription exclusively or preferentially in particular cells, tissues, or organelles. These types of regulatory elements are normally associated with genes that are expressed in a “cell-specific,” “tissue-specific,” or “organelle-specific” manner. In some alternatives, a system for editing at least one target gene in a cell is provided, wherein the system comprises a first nucleic acid sequence encoding a CRISPR guide RNA, wherein the CRISPR guide RNA is complimentary to at least one target gene in a cell and, wherein said first nucleic acid sequence is present in a vector; said system also comprising a second nucleic acid sequence encoding a Cas9 protein, a third nucleic acid sequence encoding a first adenoviral protein, and a fourth nucleic acid sequence encoding a second adenoviral protein. In some alternatives, the first, second, third and fourth nucleic acid sequences are joined to regulatory elements that are operable in a eukaryotic cell, such as a human cell.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, or enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

As used herein “upstream” have its plain and ordinary meaning when read in light of the specification, and may include but is not limited to, for example, positions 5′ of a location on a polynucleotide, and positions toward the N-terminus of a location on a polypeptide. As used herein “downstream” refers to positions 3′ of a location on nucleotide, and positions toward the C-terminus of a location on a polypeptide.

The term “construct,” as used herein, has its plain and ordinary meaning when read in light of the specification, and may include but is not limited to, for example, a recombinant nucleic acid that has been generated for the purpose of the expression of a specific nucleotide sequence(s), or that is to be used in the construction of other recombinant nucleotide sequences.

A “promoter” is a nucleotide sequence that directs the transcription of a structural gene. In some alternatives, a promoter is located in the 5′ non-coding region of a gene, proximal to the transcriptional start site of a structural gene. Sequence elements within promoters that function in the initiation of transcription are often characterized by consensus nucleotide sequences. These promoter elements include RNA polymerase binding sites, TATA sequences, CAAT sequences, differentiation-specific elements (DSEs; McGehee et al., Mol. Endocrinol. 7:551 (1993); incorporated by reference in its entirety herein), cyclic AMP response elements (CREs), serum response elements (SREs; Treisman, Seminars in Cancer Biol. 1:47 (1990); incorporated by reference in its entirety herein), glucocorticoid response elements (GREs), and binding sites for other transcription factors, such as CRE/ATF (O'Reilly et al., J. Biol. Chem. 267:19938 (1992)), AP2 (Ye et al., J. Biol. Chem. 269:25728 (1994)), SP1, cAMP response element binding protein (CREB; Loeken, Gene Expr. 3:253 (1993)) and octamer factors (see, in general, Watson et al., eds., Molecular Biology of the Gene, 4th ed. (The Benjamin/Cummings Publishing Company, Inc. 1987), and Lemaigre and Rousseau, Biochem. J. 303:1 (1994); all references incorporated by reference in their entireties herein). As used herein, a promoter may be constitutively active, repressible or inducible. If a promoter is an inducible promoter, then the rate of transcription increases in response to an inducing agent. In contrast, the rate of transcription is not regulated by an inducing agent if the promoter is a constitutive promoter. Repressible promoters are also known. In some alternatives, a regulatory element can be an untranslated region. In some alternatives, an untranslated region is a 5′ untranslated region. In some alternatives, an untranslated region is a 3′ untranslated region. In some alternatives, either 5′ or 3′ untranslated region is used. In some alternatives, both 5′ and 3′ untranslated regions are used. One skilled in the art will understand the meaning of an untranslated region as used in the alternatives here. In some alternatives, the promoter described herein can be an MND promoter. An MND promoter derives its name from myeloproliferative sarcoma virus enhancer, negative control region deleted, dl587rev primer-binding site substituted, and is a gamma retroviral synthetic promoter that contains the U3 region of a modified MoMulv LTR with myeloproliferative sarcoma virus enhancer.

As used herein, the term “enhancer” refers to a type of regulatory element that can modulate the efficiency of transcription, regardless of the distance or orientation of the enhancer relative to the start site of transcription.

As used herein, the term “transfection” has its plain and ordinary meaning when read in light of the specification, and may include but is not limited to, for example, introduction of a nucleic acid into a host cell, such as by contacting the cell with a recombinant AAV vector as described herein. As used herein, “transient transfection” refers to the introduction of exogenous nucleic acid(s) into a host cell by a method that does not generally result in the integration of the exogenous nucleic into the genome of the transiently transfected host cell. In some alternatives, the nucleic acid is RNA. In some alternatives, the nucleic acid is DNA. In some alternatives, when the nucleic acid is RNA, the nucleic acid does not generally integrate in the genome of the transiently transfected cell. In some alternatives, when the nucleic acid is DNA, the nucleic acid can integrate in the genome of the transiently transfected cell.

As used herein, the term “vector” has its plain and ordinary meaning when read in light of the specification, and may include but is not limited to, for example, a polynucleotide construct, typically a plasmid or a virus, used to transmit genetic material to a host cell. Vectors can be, for example, viruses, plasmids, cosmids, or phage. A vector as used herein can be composed of either DNA or RNA. In some alternatives, a vector is composed of DNA. An “expression vector” is a vector that is capable of directing the expression of a protein encoded by one or more genes carried by the vector when it is present in the appropriate environment. Vectors are preferably capable of autonomous replication. Typically, an expression vector comprises a transcription promoter, a gene, and a transcription terminator. Gene expression is usually placed under the control of a promoter, and a gene is said to be “operably linked to” the promoter.

As used herein “AAV system” or “AAV expression system” have their plain and ordinary meaning when read in light of the specification, and may include but is not limited to, for example, nucleic acids for expressing at least one transcript-encoding nucleic acid, and which are disposed on one or more AAV vectors. As used herein, “activity-dependent expression” (and variations of this root term) refers to nucleic acid expression that will be induced upon a change in a particular type of activity of a cell containing the nucleic acid, for example depolarization of the cell. In some alternatives, the cell is a neuron, and depolarization of the neuron in response to a stimulus induces “activity-dependent” nucleic acid expression. In some alternatives, an AAV vector includes a sequence as set forth in SEQ ID NOs: 15, 16, or 17.

The term “host cell” as described herein, has its plain and ordinary meaning when read in light of the specification, and may include but is not limited to, for example, a cell that is introduced with Cas9-mRNA/AAV-guide RNA according to the present alternatives, as well as, cells that are provided with the systems herein. Host cells can be prokaryotic cells or eukaryotic cells. Examples of prokaryotic host cells include, but are not limited to E. coli, nitrogen fixing bacteria, Staphylococcus aureus, Staphylococcus albus, Lactobacillus acidophilus, Bacillus anthracis, Bacillus subtilis, Bacillus thuringiensis, Clostridium tetani, Clostridium botulinum, Streptococcus mutans, Streptococcus pneumoniae, mycoplasmas, and/or cyanobacteria. Examples of eukaryotic host cells include, but are not limited to, protozoa, fungi, algae, plant, insect, amphibian, avian and mammalian cells. In some alternatives, a system for editing at least one target gene in a cell is provided, wherein the cell is a eukaryotic cell. In some alternatives, the cell is a mammalian cell. In some alternatives, the cell is a human cell. In some alternatives, the cell is a primary cell. In some alternatives, the cell is not a transformed cell. In some alternatives, the cell is a primary lymphocyte. In some alternatives, the cell is a primary lymphocyte, a CD34⁺ stem cell, a hepatocyte, a cardiomyocyte, a neuron, a glial cell, a muscle cell or an intestinal cell.

“Prokaryotic” cells lack a true nucleus. Examples of prokaryotic cells are bacteria (e.g., cyanobacteria, Lactobacillus acidophilus, Nitrogen-Fixing Bacteria, Helicobacter pylori, Bifidobacterium, Staphylococcus aureus, Bacillus anthrax, Clostridium tetani, Streptococcus pyogenes, Staphylococcus pneumoniae, Klebsiella pneumoniae and/or Escherichia coli) and/or archaea (e.g., Crenarchaeota, Euryarchaeota, and/or Korarchaeota). The Cas9 protein described herein is a protein from a prokaryotic cell.

“Eukaryotic” cells include, but are not limited to, algae cells, fungal cells (such as yeast), plant cells, animal cells, mammalian cells, or human cells (e.g., T-cells).

“T cell precursors” as described herein, has its plain and ordinary meaning when read in light of the specification, and may include but is not limited to, for example, lymphoid precursor cells that can migrate to the thymus and become T cell precursors, which do not express a T cell receptor. All T cells originate from hematopoietic stem cells in the bone marrow. Hematopoietic progenitors (lymphoid progenitor cells) from hematopoietic stem cells populate the thymus and expand by cell division to generate a large population of immature thymocytes. The earliest thymocytes express neither CD4 nor CD8, and are therefore classed as double-negative (CD4⁻CD8⁻) cells. As they progress through their development, they become double-positive thymocytes (CD4⁺CD8⁺), and finally mature to single-positive (CD4⁺CD8⁻ or CD4⁻CD8⁺) thymocytes that are then released from the thymus to peripheral tissues.

“Hematopoietic stem cells” or “HSC” as described herein, have their plain and ordinary meaning when read in light of the specification, and may include but is not limited to, for example, precursor cells that can give rise to myeloid cells such as, for example, macrophages, monocytes, macrophages, neutrophils, basophils, eosinophils, erythrocytes, megakaryocytes/platelets, dendritic cells or lymphoid lineages (such as, for example, T-cells, B-cells, NK-cells). HSCs have a heterogeneous population in which three classes of stem cells exist, which are distinguished by their ratio of lymphoid to myeloid progeny in the blood (L/M). In some alternatives, the cells provided are HSC cells. In some alternatives, the cell is a primary lymphocyte or a CD34⁺ stem cell.

As used herein, “autologous” refers to the donor and recipient of the stem cells being the same, for example, the patient or subject is the source of the cells.

“Primary human cells” as described herein, are directly cultured from their source organ tissue or blood cells. Compared to immortalized cell lines, primary human cells provide enhanced replication of in vivo. In some alternatives, the cells provided are primary human cells.

As used herein, the term “co-delivery” as described herein, has its plain and ordinary meaning when read in light of the specification, and may include but is not limited to, for example, delivery of two or more separate chemical entities, whether in vitro or in vivo. Co-delivery refers to the simultaneous delivery of separate agents; to the simultaneous delivery of a mixture of agents; as well as to the delivery of one agent followed by delivery of a second agent or additional agents. In all cases, agents that are co-delivered are intended to work in conjunction with each other. In some alternatives, for example, co-delivery comprises delivery of an mRNA of interest and an AAV vector.

The term “endonuclease” as described herein, has its plain and ordinary meaning when read in light of the specification, and may include but is not limited to, for example, enzymes that cleave the phosphodiester bond within a polynucleotide chain. The polynucleotide may be double-stranded DNA (dsDNA), single-stranded DNA (ssDNA), RNA, double-stranded hybrids of DNA and RNA, or synthetic DNA (for example, containing bases other than A, C, G, and T). An endonuclease may cut a polynucleotide symmetrically, leaving “blunt” ends, or in positions that are not directly opposing, creating overhangs, which may be referred to as “sticky ends.” The methods and compositions described herein may be applied to cleavage sites generated by endonucleases. In some alternatives of the system, the system can further provide nucleic acids that encode an endonuclease, including zinc finger nucleases (ZFNs), TAL effector nucleases (TALENs), meganucleases (such as MegaTALs), or CRISPR/Cas9 or a fusion protein comprising a domain of an endonuclease, for example, Cas9, TALEN, or MegaTAL, or one or more portion thereof. These examples are not meant to be limiting and other endonucleases and alternatives of the system and methods comprising other endonucleases and variants and modifications of these exemplary alternatives are possible without undue experimentation.

The term “transcription activator-like (TAL) effector nuclease” (TALEN) as described herein, has its plain and ordinary meaning when read in light of the specification, and may include but is not limited to, for example, a nuclease comprising a TAL-effector domain fused to a nuclease domain. TAL-effector DNA binding domains may be engineered to bind to a desired target and fused to a nuclease domain, such as the Fokl nuclease domain, to derive a TAL effector domain-nuclease fusion protein. The methods and systems described herein may be applied to cleavage sites generated by TAL effector nucleases. In some alternatives of the systems provided herein, the systems can further comprise a TALEN nuclease or a vector or nucleic acid encoding a TALEN nuclease. In some alternatives of the methods provided herein, the method can further comprise providing a nuclease, such as a TALEN nuclease.

MegaTALs are derived from the combination of two distinct classes of DNA targeting enzymes. Meganucleases (also referred to as homing endonucleases) are single peptide chains that have the feature of both DNA recognition and nuclease functions in the same domain. In some alternatives of the systems provided herein, the systems can further comprise a MegaTAL nuclease or a vector or nucleic acid encoding a MegaTAL nuclease. In some alternatives of the methods provided herein, the methods can further comprise providing MegaTAL nuclease or a vector or nucleic acid encoding a MegaTAL nuclease.

CRISPRs (clustered regularly interspaced short palindromic repeats) are segments of prokaryotic DNA containing short repetitions of base sequences. Each repetition is followed by short segments of “spacer DNA” from previous exposures to a bacterial virus or plasmid.

Cas9 (CRISPR associated protein 9) is an RNA-guided DNA endonuclease enzyme associated with the CRISPR adaptive immunity system in Streptococcus pyogenes, among other bacteria. S. pyogenes utilizes Cas9 to memorize and later interrogate and cleave foreign DNA, such as invading bacteriophage DNA or plasmid DNA. Cas9 performs this interrogation by unwinding foreign DNA and checking for complementarity to the 20 base pair spacer region of the guide RNA. If the DNA substrate is complementary to the guide RNA, Cas9 cleaves the invading DNA.

The CRISPR/Cas system as described herein, is used for gene editing (adding, disrupting, or changing the sequence of specific genes) and gene regulation. By delivering the Cas9 protein, a derivative, or fragment thereof and appropriate guide RNAs into a cell, the organism's genome can be cut at any desired location. It can be possible to use CRISPR to build RNA-guided genes capable of altering the genomes of entire populations. The basic components of CRISPR/Cas9 system comprise a target gene, a guide RNA, and a Cas9 endonuclease, derivative, or fragment thereof. An important aspect of applying CRISPR/Cas9 for gene editing is the need for a system to deliver the guide RNAs efficiently to a wide variety of cell types. This could for example involve delivery of an in vitro generated guide RNA as a nucleic acid (the guide RNA generated by in vitro transcription or chemical synthesis). In some alternatives the nucleic acid encoding the guide RNA is rendered nuclease resistant by incorporation of modified bases, such as 2′O-methyl bases. In some alternatives, the CRISPR/Cas9 system described herein, whereby the polynucleotide encoding the Cas9 nuclease or a derivative or functional fragment thereof (for example, a 20 nucleic acid sequence of an mRNA vector with Cas9) is provided with a poly(T) or poly(A) tail of a desired length and prepared in accordance with the teachings described herein, for example, is provided with a guide RNA that comprises one or more modified bases, such as any one or more of the modified bases described herein.

In some alternatives, the use of chemically modified guide RNAs is contemplated. Chemically-modified guide RNAs have been used in CRISPR-Cas genome editing in human primary cells (Hendel, A. et al., Nat Biotechnol. 2015 September; 33(9):985-9). Chemical modifications of guide RNAs can include modifications that confer nuclease resistance. Nucleases can be endonucleases, or exonucleases, or both. Some chemical modification, without limitations, include 2′-fluoro, 2′O-methyl, phosphorothioate dithiol 3′-3′ end linkage, 2-amino-dA, 5-methyl-dC, C-5 propynyl-C, or C-5 propynyl-U, morpholino, etc. These examples are not meant to be limiting and other chemical modifications and variants and modifications of these exemplary alternatives are also contemplated.

Exemplary guide RNAs useful with the alternatives described herein, which may contain one or more of the modified bases set forth herein. In some alternatives, the guide RNAs include a sequence as set forth in SEQ ID NOs: 12 Furthermore, an important system for expressing guide RNAs in this context is based on the use of adeno-associated virus (AAV) vectors because AAV vectors are able to transduce a wide range of primary cells. AAV vectors do not cause infection and are not known to integrate into the genome. Therefore, the use of AAV vectors has the benefits of being both safe and efficacious.

The term “exonuclease” refers to enzymes that cleave phosphodiester bonds at the end of a polynucleotide chain via a hydrolyzing reaction that breaks phosphodiester bonds at either the 3′ or 5′ end. The polynucleotide may be double-stranded DNA (dsDNA), single-stranded DNA (ssDNA), RNA, double-stranded hybrids of DNA and RNA, or synthetic DNA (for example, containing bases other than A, C, G, and T). The term “5′ exonuclease” refers to exonucleases that cleave the phosphodiester bond at the 5′ end. The term “3′ exonuclease” refers to exonucleases that cleave the phosphodiester bond at the 3′ end. Exonucleases may cleave the phosphodiester bonds at the end of a polynucleotide chain at endonuclease cut sites or at ends generated by other chemical or mechanical means, such as shearing (for example by passing through fine-gauge needle, heating, sonicating, mini bead tumbling, or nebulizing), ionizing radiation, ultraviolet radiation, oxygen radicals, chemical hydrolysis or chemotherapy agents. Exonucleases may cleave the phosphodiester bonds at blunt ends or sticky ends. E. coli exonuclease I and exonuclease III are two commonly used 3′-exonucleases that have 3′-exonucleolytic single-strand degradation activity. Other examples of 3′-exonucleases include Nucleoside diphosphate kinases (NDKs), NDK1 (NM23-H1), NDK5, NDK7, and NDK8 (Yoon J-H, et al., Characterization of the 3′ to 5′ exonuclease activity found in human nucleoside diphosphate kinase 1 (NDK1) and several of its homologues. (Biochemistry 2005:44(48):15774-15786.), WRN (Ahn, B., et al., Regulation of WRN helicase activity in human base excision repair. J. Biol. Chem. 2004, 279:53465-53474) and Three prime repair exonuclease 2 (Trex2) (Mazur, D. J., Perrino, F. W., Excision of 3′ termini by the Trex1 and TREX2 exonucleases. Characterization of the recombinant proteins. J. Biol. Chem. 2001, 276:17022-17029; both references incorporated by reference in their entireties herein). E. coli exonuclease VII and T7-exonuclease Gene 6 are two commonly used 5′-3′ exonucleases that have 5% exonucleolytic single-strand degradation activity. The exonuclease can be originated from prokaryotes, such as E. coli exonucleases, or eukaryotes, such as yeast, worm, murine, or human exonucleases. In some alternatives of the systems provided herein, the systems can further comprise an exonuclease or a vector or nucleic acid encoding an exonuclease. In some alternatives, the exonuclease is Trex2. In some alternatives of the methods provided herein, the methods can further comprise providing exonuclease or a vector or nucleic acid encoding an exonuclease, such as Trex2.

As used herein, a “guide” as described herein, has its plain and ordinary meaning when read in light of the specification, and may include but is not limited to, for example, any polynucleotide that site-specifically guides a nuclease to a target nucleic acid sequence. In some alternatives, a guide comprises RNA, DNA, or combinations of RNA and DNA. Exemplary guide RNAs useful with the alternatives described herein, which may contain one or more of the modified bases set forth herein are provided in sequence as set forth in SEQ ID NO: 12.

A “genomic region” is a segment of a chromosome in the genome of a host cell that is present on either side of the target nucleic acid sequence site or, alternatively, also comprises a portion of the target site. The homology arms of the donor polynucleotide have sufficient homology to undergo homologous recombination with the corresponding genomic regions. In some alternatives, the homology arms of the donor polynucleotide share significant sequence homology to the genomic region immediately flanking the target site; it is recognized that the homology arms can be designed to have sufficient homology to genomic regions farther from the target site.

As used herein, “non-homologous end joining” (NHEJ) as described herein, have their plain and ordinary meaning when read in light of the specification, and may include but is not limited to, for example, repair of a DSB in DNA by direct ligation of one end of the break to the other end of the break without a requirement for a donor polynucleotide. NHEJ is a DNA repair pathway available to cells to repair DNA without the use of a repair template. NHEJ in the absence of a donor polynucleotide often results in nucleotides being randomly inserted or deleted at the site of the DSB.

As used herein “cleavage site” refers to a sequence that mediates the separation of a first polypeptide that would otherwise be in cis to a second polypeptide. Accordingly, for simplicity, “cleavage,” “cleavage site,” and the like as used herein refer to the separation of any two polypeptides that are encoded by a single polynucleotide in cis. Thus, “cleavage” and “cleavage site,” can, but do not necessarily refer to proteolytic sites and events, and can also refer to other mechanisms for mediating the separation of polypeptides, for example ribosomal skipping. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. Double stranded DNA, RNA, or DNA/RNA hybrid cleavage can result in the production of either blunt ends or staggered ends.

The term “complementary to” means that the complementary sequence is homologous to all or one or more portions of a reference polynucleotide sequence. For illustration, the nucleotide sequence “CATTAG” corresponds to a reference sequence “CATTAG” and is complementary to a reference sequence “GTAATC.”

As used herein, the term “label” refers to a detectable molecule. A number of suitable labels comprise polypeptides. As such, as used herein, a “label nucleic acid” refers to a nucleic acid encoding a label. In some alternatives, the AAV vector systems comprise a label polynucleotide. Thus, in some alternatives, a promoter (such as an MND promoter) is operatively linked to a label polynucleotide, such that the AAV vectors described herein comprise a reporter. Example labels that are suitable in accordance with alternatives herein include, but are not limited to, green fluorescent protein (GFP), including, for example, Aequoria victoria GFP, Renilla muelleri GFP, Renilla reniformis GFP, Renilla ptilosarcus GFP, blue fluorescent protein (BFP), yellow fluorescent protein (YFP), red fluorescent protein (RFP), cyan fluorescent protein (CFP), or orange fluorescent proteins (OFP). Additional reporter genes include, but are not limited to neomycin, phosphoro-transferase, chloramphenicol acetyl transferase, thymidine kinase, luciferase, β-glucuronidase, aminoglycoside, phosphotransferase, hygromycin B, xanthine-guanine phosphoribosyl, luciferases (e.g., renilla, firefly, etc.), DHFR/methotrexate, β-galactosidase, alkaline phosphatase, turbo and/or tagRFP, or nuclear targeted versions of any of the aforementioned reporter genes. In some alternatives, the polypeptide of interest comprises the label itself, for example when production of label in active cells is desired. In some alternatives, an AAV construct provided herein comprises an MND promoter driven GFP cassette and wherein the MND promoter driven GFP cassette provides for tracking of AAV transduction efficiency.

The term “gene expression” as described herein, has its plain and ordinary meaning when read in light of the specification, and may include but is not limited to, for example, biosynthesis of a gene product. For example, in the case of a structural gene, gene expression involves transcription of the structural gene into mRNA and the translation of mRNA into one or more polypeptides.

A “polypeptide” is a polymer of amino acid residues joined by peptide bonds, whether produced naturally or synthetically. Polypeptides of less than about 10 amino acid residues are commonly referred to as “peptides.” A polypeptide can be considered as a protein.

A “protein” is a macromolecule comprising one or more polypeptide chains. A protein may also comprise non-peptide components, such as carbohydrate groups. Carbohydrates and other non-peptide substituents may be added to a protein by the cell in which the protein is produced, and will vary with the type of cell. Proteins are defined herein in terms of their amino acid backbone structures; substituents such as carbohydrate groups are generally not specified, but may be present nonetheless. In some alternatives, a system for editing at least one target gene in a cell is provided, wherein the system comprises a first nucleic acid sequence encoding a CRISPR guide RNA, wherein the CRISPR guide RNA is complimentary to at least one target gene in a cell and, wherein said first nucleic acid sequence is present in a vector; said system also comprising a second nucleic acid sequence encoding a Cas9 protein, a third nucleic acid sequence encoding a first adenoviral protein and a fourth nucleic acid sequence encoding a second adenoviral protein. Amino acid sequences may be described herein with a SEQ ID NO, and are described throughout the application and included in Appendix I. In some alternatives, an amino acid sequence described herein is 85%, 86%, 87%, 88%, 89%, 90%, 91%, 92%, 93%, 94%, 95%, 96%, 97%, 98%, 99% or 100% identical, or is within a range defined by any two of the aforementioned percentages, to a sequence of any one of SEQ ID NOs: 10-11.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to alternatives containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

### X-Linked Hyper IgM Syndrome

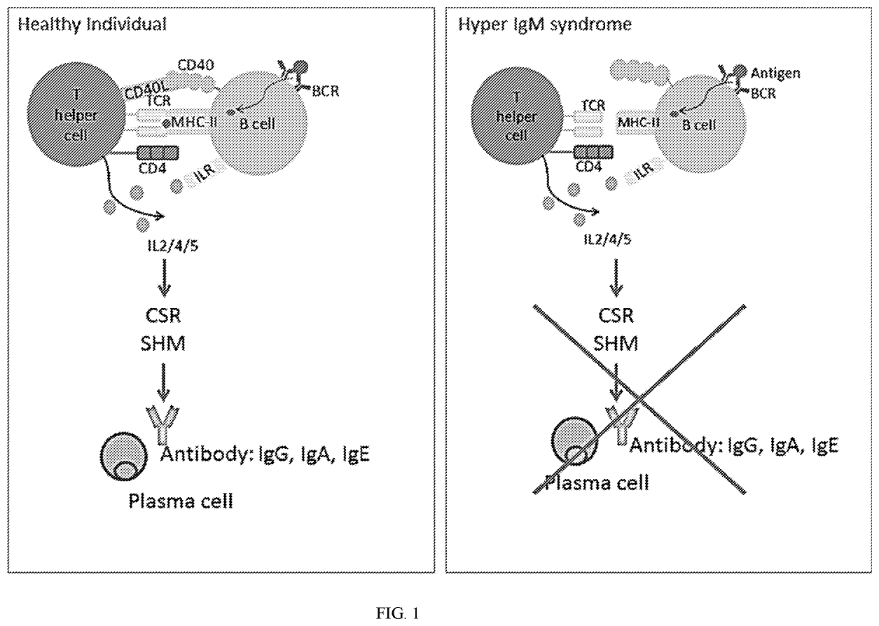

X-linked hyper-immunoglobulin M (IgM) syndrome (X-HIGM) arises in humans carrying mutations in CD40LG, and is characterized by recurrent infections, low serum immunoglobulins G, A, and E (IgG, IgA, and IgE) with normal or elevated IgM levels, reduced numbers of memory B cells, and the absence of class-switched memory B cells (FIG. 1). A genetic therapy that could reconstitute CD40L function to the appropriate hematopoietic lineages could greatly improve treatment options for these patients.

Interestingly, studies of female carriers of X-HIGM have shown that X-inactivation of the CD40LG gene is random and highly heterogeneous, with some carriers expressing non-mutated CD40L in only 5-10% of T cells. Also, a female carrier of X-HIGM with non-mutated CD40L expressed on only 5% of T cells displayed symptoms characteristic of hyper-IgM syndrome. Female carriers with non-mutated CD40L expressed in as few as 12% of T cells display no symptoms, suggesting a threshold may exist between 5-12% for non-mutated CD40L expression necessary for non-compromised immune function. Thus, a gene therapy may provide clinical benefit even with a relatively low percentage of corrected cells (Hollenbaugh, D., et al., The Journal of clinical investigation, 1994. 94(2): p. 616-622; de Saint Basile, G., et al., European journal of immunology, 1999. 29(1): p. 367-373).

CD40:CD40 ligand (CD40L) co-stimulatory signals play an essential role in cross-talk between B cells and T cells upon antigen recognition (Banchereau, J., et al., Annual review of immunology, 1994. 12(1): p. 881-926; Foy, T. M., et al., Annual review of immunology, 1996. 14(1): p. 591-617; Elgueta, R., et al., Immunol Rev, 2009. 229(1): p. 152-72). CD40L is expressed primarily on activated T cells, but is also expressed by other immune cells such as dendritic cells and macrophages under inflammatory conditions, while CD40 is constitutively expressed on B cells (Schonbeck, U. and P. Libby, The CD40/CD154 receptor/ligand dyad. Cellular and Molecular Life Sciences CMLS, 2001. 58(1): p. 4-43; van Kooten, C. and J. Banchereau, Current opinion in immunology, 1997. 9(3): p. 330-337). Activation of CD40 by CD40L is an essential step of B cell development, promoting B cell proliferation and acting as a critical checkpoint for immunoglobulin class switching and somatic hypermutation (Clark, E. A. and J.A. Ledbetter, Nature, 1994. 367(6462): p. 425; Bishop, G. A. and B. S. Hostager, Cytokine & growth factor reviews, 2003. 14(3-4): p. 297-309; Crotty, S., Nature Reviews Immunology, 2015. 15(3): p. 185; Kawabe, T., et al., Immunity, 1994. 1(3): p. 167-178).

A previous attempt to develop a genetic therapy for X-HIGM used a γ-retrovirus to deliver a CD40L cDNA expression cassette to bone marrow hematopoietic stem cells (HSCs) of Cd40l−/− mice. However, this resulted in T cell lymphoproliferative disorders in the majority of mice, suggesting that endogenous transcriptional regulation of CD40L could be an important safety consideration (Sacco, M. G., et al., Cancer gene therapy, 2000. 7(10): p. 1299; Brown, M. P., et al., Nature medicine, 1998. 4(11): p. 1253).

Previous work related to a gene editing approach for delivery a CD40L coding sequence directly downstream of the endogenous promoter in primary human T cells (Hubbard, N., et al., Blood, 2016. 127(21): p. 2513-22). This approach was based on homology-directed repair (HDR) using CD40LG-specific TALE-nucleases (TALEN) and adeno-associated virus serotype 6 (AAV6)-delivered CD40L cDNA with CD40LG homology arms. Expression kinetics of CD40L in gene-edited T cells mirrored that of the endogenous protein in unedited T cells. Further, gene editing rescued CD40L expression and function in X-HIGM patient T cells in vitro. While promising as a T cell therapy, the methods described in Hubbard do not translate to a gene editing approach using hematopoietic stem and progenitor cells (HSPCs) as a potential curative treatment for X-HIGM.

Compared to T cells, HDR-based gene editing has been difficult to achieve in CD34⁺ HSPCs (Sather, B. D., et al., Sci Transl Med, 2015. 7(307): p. 307ra156). Furthermore, gene-edited HSPCs engraft poorly into immunodeficient humanized mice in comparison to unedited cells (De Ravin, S. S., et al., Nat Biotechnol, 2016. 34(4): p. 424-9; Dever, D. P., et al., Nature, 2016. 539(7629): p. 384-389; De Ravin, S. S., et al., Sci Transl Med, 2017. 9(372): p. eaah3480; Hoban, M. D., et al., Blood, 2015. 125(17): p. 2597-604). Detecting high rates of persistent edited cells has been particularly challenging using HSPCs from G-CSF mobilized peripheral blood, a more clinically relevant source.

Accordingly, some alternatives provided herein relate to treating, ameliorating, inhibiting, or improving X-HIGM using a therapeutic genome editing approach. In some alternatives, systems and methods for the introduction of an intact CD40LG cDNA under control of the endogenous promoter and enhancer in HSPCs are provided. In some alternatives, the systems and methods described herein rescue immunologic and functional defects in CD40L and provide a curative therapy.

Some alternatives, for example, relate to a homology directed repair-based gene editing approach in CD34⁺ hematopoietic stem cells, wherein a cDNA encoding CD40L is under control of the endogenous promoter. On-target integration of CD40L or control GFP coding sequences in up to 30% of human peripheral blood stem cells. To allow assessment of the engraftment potential of edited cells, GFP-expressing CD34⁺ cells were transplanted into immunodeficient mice. The transplanted human cells constituted a substantial proportion of the cells recovered from the mouse bone marrows, with 1.5% of them containing the desired edit. The recovered edited cells included cells of different lineages including myeloid, B, and CD34⁺ cells. The addition of small molecules to this ex-vivo editing protocol increased human cell engraftment, but there was no impact on the percentage of engrafted cells containing the desired edit. These findings demonstrate editing of the CD40LG locus in human hematopoietic stem cells.

Furthermore, the disclosure provided herein relates to methods, systems, and compositions for efficient culturing and editing the CD40LG locus of human peripheral blood HSPCs, and the engraftment of edited pluripotent stem cells in immunodeficient mice.

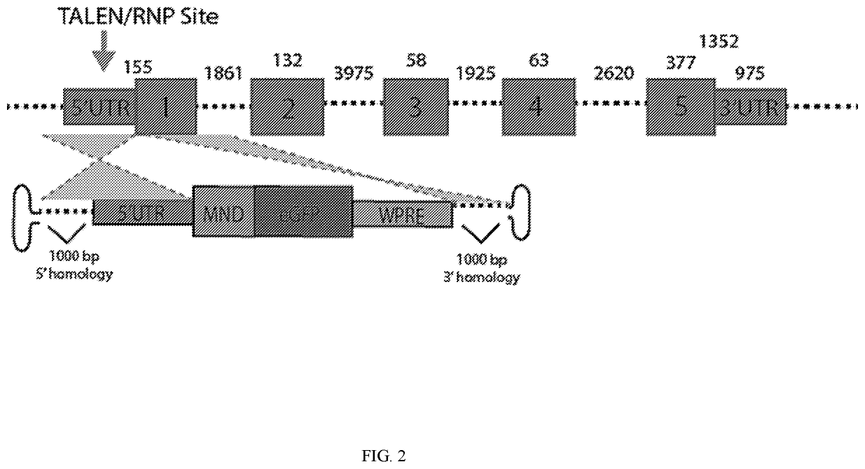

In some alternatives, the efficiency of AAV-assisted HDR in human hematopoietic stem cells (HSCs) is shown between two nuclease platforms: Cas9 RNP and TALEN. The RNP guide RNA (gRNA) was designed to target Cas9 cleavage to within 15 bp of that of the TALEN in order to compare the nucleases using the same donor template. Because the CD40LG promoter is not active in HSCs, the AAV6 donor was designed to deliver an MND promoter-GFP expression cassette with 1 kb of CD40LG homology on either side (FIG. 2) (Challita, P.-M., et al., Journal of virology, 1995. 69(2): p. 748-755). The CD40LG homology arms in the targeting construct had the TALEN and Cas9 gRNA binding sites deleted, rendering it non-cleavable by both nucleases.

Some alternatives concern methods for using adult mobilized CD34⁺ cells and co-delivery of either TALEN mRNA or Cas9/gRNA ribonucleoprotein complexes (RNPs) and an AAV donor for targeted integration of a promoter-driven fluorescent marker. In some alternatives, the methods provided herein achieve efficient homology directed repair rates across multiple donors at an efficiency of 10%, 15%, 20%, 25%, 30%, 35%, 40%, 45%, 50%, 60%, 70%, 80%, or greater, or an efficiency within a range defined by any two of the aforementioned values for TALEN and an efficiency of 10%, 15%, 20%, 25%, 30%, 35%, 40%, 45%, 50%, 60%, 70%, 80%, or greater, or an efficiency within a range defined by any two of the aforementioned values for RNP. In some alternatives, the highest levels of cell viability is observed using RNP/AAV co-delivery. In some alternatives, edited HSC retain their potential to give rise to multiple lineages in colony forming unit assays. In some alternatives, the systems provided herein provide long-term engraftment and differentiation potential in immune-deficient mice. In some alternatives, AAV vectors carrying CD40LG cDNA restore expression in CD40LG deficient cells. In some alternatives, the systems and methods described herein provide therapeutic correction of the disease or a disease symptom in patients.

### Homology Directed Repair

Homology directed repair (HDR), refers to the process of repairing DNA damage using a homologous nucleic acid (e.g., a sister chromatid or an exogenous nucleic acid). In a normal cell, HDR typically involves a series of steps such as recognition of the break, stabilization of the break, resection, stabilization of single stranded DNA, formation of a DNA crossover intermediate, resolution of the crossover intermediate, and ligation. As described herein, HDR can be used to alter a target sequence and correct (e.g., repair or edit) a mutation in the genome. While not wishing to be bound by theory, it is believed that alteration of the target sequence occurs by HDR with a donor template or template nucleic acid. For example, the donor template or the template nucleic acid provides for alteration of the target position.

Some alternatives provided herein relate to methods and systems for homology directed repair of the gene associated with X-HIGM. In some alternatives, the gene is a CD40LG gene. In some alternatives, the method comprises HDR of the CD40LG gene in human hematopoietic cells. In some alternatives, the method and systems include nuclease-based HDR of the CD40LG gene. In some alternatives, the nuclease based HDR comprises a TALEN based nuclease. In some alternatives, the nuclease based HDR comprises a CRISPR/Cas based nuclease.

### TALEN

Transcription activator-like (TAL) effector-DNA modifying enzyme (TALEN) is a restriction enzyme that can be engineered to cut specific sequences of DNA. TALENs are made by fusing a TAL-effector domain to a DNA cleavage domain.

In some alternatives, the CD40LG locus used in targeting with CD40LG TALENs is co-delivered with an AAV donor.

In some alternatives the CD40LG TALEN forward sequence is defined by SEQ ID NO: 10 and 21. In some alternatives, the CD40LG TALEN reverse sequence is defined by SEQ ID NO: 11 and 22. In some alternatives, a forward sequence and/or the reverse sequence is 85%, 86%, 87%, 88%, 89%, 90%, 91%, 92%, 93%, 94%, 95%, 96%, 97%, 98%, 99% or 100% identical, or is within a range defined by any two of the aforementioned percentages, to a sequence of any one of SEQ ID NO: 10, 21, 11, or 22.

In some alternatives, the AAV donor comprises a GFP cassette under control of an MND promoter. In some alternatives, the AAV donor has a 1 kb homology arm flanking an MND promoter driven GFP cassette (SEQ ID NO: 14). In some alternatives, the AAV donor comprises one or more nucleotide mutations to abolish cleavage by TALENs and guide sequences as set forth in SEQ ID NOs: 14 and 15. In some alternatives, a nucleotide sequence of the MND promoter is 85%, 86%, 87%, 88%, 89%, 90%, 91%, 92%, 93%, 94%, 95%, 96%, 97%, 98%, 99% or 100% identical, or is within a range defined by any two of the aforementioned percentages, to a sequence of any one of SEQ ID NOs: 14 or 15.

Some alternatives provided herein relate to a TALEN nuclease for use in HDR of a CD40LG gene (SEQ ID NO: 13). In some alternatives, the TALEN binds to a TALEN binding site in the CD40LG gene. In some alternatives, a CD40LG TALEN binds to native CD40LG sequence (SEQ ID NO: 13). In some alternatives, a nucleotide sequence of the CD40LG gene is 85%, 86%, 87%, 88%, 89%, 90%, 91%, 92%, 93%, 94%, 95%, 96%, 97%, 98%, 99% or 100% identical, or is within a range defined by any two of the aforementioned percentages, to a sequence of SEQ ID NO: 13.

The CD40LG locus used in targeting with CD40LG TALENs comprises the following components from 5′ to 3′: upstream homology arm (SEQ ID NO: 18); exon 1 (SEQ ID NO: 26), including a guide RNA (SEQ ID NO: 12); T-for (TALEN forward binding site; SEQ ID NO: 23); cleavage site (SEQ ID NO: 25); T-rev (TALEN reverse binding site; SEQ ID NO: 24); exon 2 (SEQ ID NO: 27); and downstream homology arm (SEQ ID NO: 19). In some alternatives, a nucleotide sequence of any one of the aforementioned components (including any one or more of the upstream homology arm, exon 1, guide RNA, T-for, cleavage site, T-rev, exon 2, or downstream homology arm) is 85%, 86%, 87%, 88%, 89%, 90%, 91%, 92%, 93%, 94%, 95%, 96%, 97%, 98%, 99% or 100% identical, or is within a range defined by any two of the aforementioned percentages, to a sequence of any one of the respective SEQ ID NOs, including SEQ ID NO: 18, SEQ ID NO: 26, SEQ ID NO: 12, SEQ ID NO: 23, SEQ ID NO: 25, SEQ ID NO: 24, SEQ ID NO: 27, and SEQ ID NO: 19 respectively.

In some alternatives, the CD40LG locus used in targeting with CD40LG TALENs is co-delivered with an AAV donor. In some alternatives the CD40LG TALEN forward sequence is defined by SEQ ID NO: 10. In some alternatives, the CD40LG TALEN reverse sequence is defined by SEQ ID NO: 11. In some alternatives, the AAV donor comprises a GFP cassette under control of an MND promoter. In some alternatives, the AAV donor has a 1 kb homology arm flanking an MND promoter driven GFP cassette (SEQ ID NO: 14). In some alternatives, the AAV donor comprises one or more nucleotide mutations to abolish cleavage by TALENs and guide sequences as set forth in SEQ ID NOs: 14 and 15.

### CRISPR/Cas

Some alternatives provided herein relate to a Cas nuclease for use in HDR of a gene of interest. In some alternatives, the Cas nuclease is a Cas9 nuclease. Cas9 is an RNA-guided DNA endonuclease enzyme associated with the CRISPR (Clustered Regularly Interspersed Palindromic Repeats) adaptive immunity system in Streptococcus pyogenes, among other bacteria. S. pyogenes utilizes Cas9 to memorize and later interrogate and cleave foreign DNA, such as invading bacteriophage DNA or plasmid DNA. Cas9 performs this interrogation by unwinding foreign DNA and checking for if it is complementary to the 20 base pair spacer region of the guide RNA. If the DNA substrate is complementary to the guide RNA, Cas9 cleaves the invading DNA.

In some alternatives, the Cas nuclease is delivered in a complex with a single guide RNA as a ribonucleoprotein complex (RNP). In some alternatives, the CRISPR guide sequence is defined by SEQ ID NO: 12. In some alternatives, the RNP is co-delivered with an AAV donor. In some alternatives, the AAV donor is a self-complementary AAV (scAAV). In some alternatives, the AAV donor comprises a GFP cassette under control of an MND promoter wherein a protospacer adjacent motif (PAM) site is deleted.

In some alternatives, the nucleic acid includes a single guide RNA (sgRNA) such as one that is encoded by SEQ ID NO: 12. In some alternatives, the nucleotide sequence encoding the sgRNA is 85%, 86%, 87%, 88%, 89%, 90%, 91%, 92%, 93%, 94%, 95%, 96%, 97%, 98%, 99% or 100% identical, or within a range defined by any two of the aforementioned percentages, to a sequence of SEQ ID NO: 12.

### Cells

Some alternatives provided herein relate to co-delivery of a nuclease, such as a TALEN or Cas nuclease, and an AAV donor template to modify endogenous CD40LG locus in a cell. In some alternatives, the cell is a mammalian cell. In some alternatives, the cell is a human cell. In some alternatives, the cell is an autologous cell. In some alternatives, the cell is a primary cell. In some alternatives, the cell is a lymphocyte. In some alternatives, the cell is not a transformed cell. In some alternatives, the cell is a primary lymphocyte. In some alternatives, the cell is a lymphocyte precursor cell. In some alternatives, the cell is a T cell. In some alternatives, the cell is a hematopoietic cell. In some alternatives, the cell is a CD34⁺ cell. In some alternatives, the cell is a primary human hematopoietic cell.

In some alternatives, the cell is transformed by co-delivery of a nuclease, such as a TALEN nuclease or Cas nuclease, and an AAV donor template to modify endogenous CD40LG locus in a cell. In some alternatives, a method of editing a CD40LG gene in a cell is provided, wherein the method comprises introducing into a cell a first vector that comprises a first nucleic acid sequence encoding a guide RNA, such as a TALEN guide RNA or a CRISPR guide RNA, wherein the guide RNA is complimentary to at least one target gene in said cell, and introducing into said cell a second nucleic acid sequence encoding a nuclease, such as a TALEN nuclease or a Cas nuclease, a derivative, or fragment thereof. In some alternatives, a cell is provided, wherein the cell is manufactured by the said methods. In some alternatives, the method includes providing to the cell one or more of the nucleic acid compositions described herein such as a sequence in accordance with, or encoded by, one or more of SEQ ID NOs: 1-27, or, for example, a sequence that is 85%, 86%, 87%, 88%, 89%, 90%, 91%, 92%, 93%, 94%, 95%, 96%, 97%, 98%, 99% or 100% identical, or within a range defined by any two of the aforementioned percentages, to a sequence in accordance with, or encoded by, any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, and/or 27.

### Methods of Therapy

Some alternatives provided herein relate to methods of promoting HDR of a CD40LG gene in a subject in need thereof. In some alternatives, the method comprises selecting or identifying a subject in need thereof. A selected or identified subject in need thereof is a subject that presents with symptoms of X-HIGM, or a subject that has been diagnosed with X-HIGM. Such evaluations can be made clinically or by diagnostic test.

In some alternatives, the method comprises adoptive cellular therapy or adoptive cell transfer of treated cells to a subject in need. In some alternatives, adoptive cellular therapy or adoptive cell transfer comprises administering cells for promoting homology directed repair of a CD40LG gene in a subject. In some alternatives, the method comprises obtaining cells from the subject in need thereof. In some alternatives, the cells from the subject in need are primary human hematopoietic cells. In some alternatives, the cells are transformed by co-delivery of a nuclease, such as a TALEN nuclease or a Cas nuclease, and an AAV donor, which modifies the endogenous CD40LG locus in the cell. In some alternatives, the method comprises expanding the transformed cells. In some alternatives, the method comprises selecting transformed cells that have successful modification of the CD40LG locus in the cell. In some alternatives, the transformed cells are administered to the patient.

In some alternatives, administration of the transformed cells to the patient comprises administration of autologous cells to the patient. In some alternatives, administration of the transformed cells to the patient treats, inhibits, or ameliorates symptoms of X-HIGM. In some alternatives, administration of the transformed cells to the patient treats X-HIGM. In some alternatives, the method reduces bacterial or opportunistic infections. In some alternatives, the method reduces intermittent neutropenia.

In some alternatives, an amount of treated cells is administered as a composition. In some alternatives, the amount of cells administered is 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, or 1×10⁹ cells, or greater, or an amount within a range defined by any two of the aforementioned values.

In some alternatives, the treated cells are administered to a subject as a co-therapy with an additional therapy that is used to treat the symptoms of the disorder or used to treat the disorder. In some alternatives, the additional therapy includes immunoglobulin therapy, an antibiotic therapy, an antimicrobial therapy, bone marrow stimulation therapy (such as a granulocyte-colony stimulating factor (G-CSF) therapy), bone marrow transplantation therapy, corticosteroid therapy, or transfusion therapy.

### Pharmaceutical Compositions

Cells prepared by the systems or methods provided herein can be administered directly to a patient for targeted homology directed repair of a CD40LG locus and for therapeutic or prophylactic applications, for example, for treating, inhibiting, or ameliorating X-HIGM. In some alternatives, cells are prepared by the systems provided herein. In some alternatives, a composition is provided, wherein the composition comprises the cell. In some alternatives, the compositions described herein, can be used in methods of treating, preventing, ameliorating, or inhibiting X-HIGM or ameliorating a disease condition or symptom associated with X-HIGM.

The compositions comprising the cells are administered in any suitable manner, and in some alternatives with pharmaceutically acceptable carriers. Suitable methods of administering such compositions comprising the cells are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as, by the particular method used to administer the composition. Accordingly, there is a wide variety of suitable formulations of pharmaceutical compositions that are available (see, e.g., Remington's Pharmaceutical Sciences).

Formulations suitable for parenteral administration, such as, for example, by intravenous, intramuscular, intradermal, and/or subcutaneous routes, include aqueous and/or non-aqueous, isotonic sterile injection solutions, which can contain antioxidants, buffers, bacteriostats, and/or solutes that render the formulation isotonic with the blood of the intended recipient, and/or aqueous and/or non-aqueous sterile suspensions that can include suspending agents, solubilizers, thickening agents, stabilizers, and/or preservatives. The disclosed compositions can be administered, for example, by intravenous infusion, orally, topically, intraperitoneally, intravesically or intrathecally. The formulations of compounds can be presented in unit-dose or multi-dose sealed containers, such as ampules or vials. Injection solutions and suspensions can be prepared from sterile powders, granules, and/or tablets of the kind previously described.

In some alternatives, one or more of parenteral, subcutaneous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal routes of administration are contemplated. In some alternatives, the composition to be administered can be formulated for delivery via one or more of the above noted routes.

### Certain Methods for Editing an CD40LG Gene in a Cell

Some embodiments of the methods and compositions provided herein include methods for editing an CD40LG gene in a cell. Some such embodiments, include (i) introducing a polynucleotide encoding a guide RNA (gRNA) into the cell, or introducing a polynucleotide encoding a TALEN in the cell, and (ii) introducing a template polynucleotide into the cell. In some embodiments, the CD40LG gene has at least 95% identity with the nucleotide sequence of SEQ ID NO:13.

In some embodiments, the gRNA comprises a nucleic acid having at least about 85%, 90%, 95%, 99% or 100% identity to the nucleotide sequence of SEQ ID NO:12.

In some embodiments, introducing a polynucleotide encoding a gRNA into the cell comprises contacting the cell with a ribonucleoprotein (RNP) comprising a CAS9 protein and the polynucleotide encoding the gRNA. In some embodiments, the RNP comprises the CAS9 protein and the polynucleotide encoding the gRNA having a ratio between 0.1:1 and 1:10, a ratio between 1:1 and 1:5, or a ratio of about 1:1.2.

In some embodiments, the template polynucleotide encodes at least a portion of the CD40LG gene, or complement thereof. In some embodiments, the template polynucleotide encodes at least a portion of a wild-type CD40LG gene, or complement thereof. In some embodiments, the template polynucleotide comprises at least or at least about 1 kb of the CD40LG gene. In some embodiments, the template polynucleotide comprises a nucleic acid having at least 95% identity to the nucleotide sequence of SEQ ID NO:15. In some embodiments, the template polynucleotide comprises the nucleotide sequence of SEQ ID NO:15.

In some embodiments, a viral vector comprises the template polynucleotide. In some embodiments, the vector is an adeno-associated viral (AAV) vector. In some embodiments, the vector is a self-complementary AAV (scAAV) vector.

In some embodiments, step (i) is performed before step (ii). In some embodiments, steps (i) and (ii) are performed simultaneously. In some embodiments, steps (i) and/or (ii) comprise performing nucleofection. In some embodiments, performing nucleofection comprises use of a LONZA system. In some embodiments, the system comprises use of a square wave pulse.

Some embodiments also include contacting the cell with IL-6. In some embodiments, the IL-6 has a concentration from about 20 ng/ml to about 500 mg/ml or 20 ng/ml to 500 mg/ml, from about 50 ng/ml to about 150 mg/ml or 50 ng/ml to 150 mg/ml, or of about 100 mg/ml or 100 mg/ml. In some embodiments, the IL-6 has a concentration of at least or at least about 1 ng/ml, 10 ng/ml, 50 ng/ml, 100 ng/ml, 200 ng/ml, 500 ng/ml, or a concentration within a range of any two of the foregoing concentrations.

In some embodiments, the cell is incubated in a Serum-Free Expansion Medium II (SFEMII) medium.

In some embodiments, a population of cells comprises the cell, the population having a concentration from about 1×10⁵ cells/ml to about 1×10⁶ cells/ml or 1×10⁵ cells/ml to 1×10⁶ cells/ml, from about 1×10⁵ cells/ml to about 5×10⁵ cells/ml or 1×10⁵ cells/ml to 5×10⁵ cells/ml, or from about 2.5×10⁵ cells/ml or 2.5×10⁵ cells/ml. In some embodiments, the population of cells has a density less than or less than about 2,000,000 cells/ml, 1,000,000 cells/ml, 500,000 cells/ml, 250,000 cells/ml, 100,000 cells/ml, 50,000 cells/ml, 10,000 cells/ml, 1000 cells/ml, or a density within a range of any two of the foregoing densities.

Some embodiments also include diluting the population of cells after steps (i) and (ii) are performed. In some embodiments, the population of cells is diluted about 16 hours or 16 hours after steps (i) and (ii) are performed. In some embodiments, the population of cells is diluted to about 250,000 cells/ml or 250,000 cells/ml. In some embodiments, the population of cells is diluted to less than or less than about 2,000,000 cells/ml, 1,000,000 cells/ml, 500,000 cells/ml, 250,000 cells/ml, 100,000 cells/ml, 50,000 cells/ml, 10,000 cells/ml, 1000 cells/ml, or a density within a range of any two of the foregoing densities.

Some embodiments also include contacting the cell with stem cell factor (SCF), FMS-like tyrosine kinase-3 (Flt-3), thrombopoietin (TPO), a TPO receptor agonist, UM171, or stemregenin (SR1). In some embodiments, the TPO receptor agonist comprises Eltrombopag.

In some embodiments, steps (i) and/or (ii) comprise contacting the cell with an HDM2 protein. In some embodiments, the HDM2 protein has a concentration from about 1 nM to about 50 nM or 1 nM to 50 nM, or from about 6.25 nM to about 25 nM or 6.25 nM to 25 nM. In some embodiments, the HDM2 protein has a concentration of at least or at least about 1 nM, 10 nM, 20 nM, 30 nM, 40 nM, 50 nM, 100 nM, 200 nM, 500 nM, 1000 nM, or a concentration within a range of any two of the foregoing concentrations.

In some embodiments, the cell is contacted with at least or at least about 1000 MOI of the AAV, or at least or at least about 2500 MOI of the AAV. In some embodiments, the cell or a population of cells comprising the cell, is contacted with an amount or concentration of AAV of at least or at least about 10 MOI, 100 MOI, 200 MOI, 500 MOI, 1000 MOI, 2000 MOI, 5000 MOI, or 10000 MOI, or an amount or concentration within a range of any two of the foregoing numbers.

In some embodiments, the cell is contacted with at least or at least about 100 μg/ml of the RNP, or at least or at least about 200 μg/ml of the RNP. In some embodiments, the cell is contacted with RNP having a concentration of at least or at least about 1 μg/ml, 10 μg/ml, 100 μg/ml, 200 μg/ml, 500 μg/ml, or 1000 μg/ml, or a concentration within a range of any two of the foregoing concentrations.

In some embodiments, steps (i) and/or (ii) comprise contacting about 1,000,000 cells/20 μl nucleofection reaction or 1,000,000 cells/20 μl nucleofection reaction, wherein the nucleofection reaction comprises the gRNA and/or the template polynucleotide.

In some embodiments, the nucleofection reaction is performed in a volume of at or about 10 μl, 50 μl, 100 μl, 200 μl, 500 μl, 1000 μl, 1500 μl, 2000 μl, 50000 μl, or a volume within a range of any two of the foregoing volumes.

In some embodiments, the cell is mammalian. In some embodiments, the cell is human. In some embodiments, the cell is a primary cell. In some embodiments, the cell is a hematopoietic stem cell (HSC). In some embodiments, the cell is a T cell or a B cell. In some embodiments, the cell is a CD34+ cell. In some embodiments, the cell is ex vivo.

### EXAMPLES

### Experimental Methods

Non-obese diabetic (NOD) scid gamma (NSG) mice were purchased from Jackson Laboratory. All animal studies were performed according to the Association for Assessment and Accreditation of Laboratory Animal Care (AAALAC) standards and were approved by the SCRI Institutional Animal Care and Use Committee (IACUC). Six to 10 week old mice were treated with 25 or 35 mg/kg of BUSULFEX (Henry Schein Inc.) via intraperitoneal injection, diluted 1:1 in phosphate-buffered saline. Twenty-four hours later, 2×10⁶ mock or gene edited hematopoietic stem cells in phosphate-buffered saline were delivered via retro-orbital injection. Animals were euthanized 12 to 16 weeks post-transplant, and femurs and spleens were analyzed for engraftment of human cells.

Ex vivo culture of CD34+ hematopoietic stem/progenitor cells. Cryopreserved CD34⁺ cells enriched from PBMC mobilized adult donors were obtained from the Core Center for Excellence in Hematology at the Fred Hutchinson Cancer Research Center. Cells were thawed and plated at 1×10⁶ cells/ml in serum-free stem cell growth media [CellGenix GMP SCGM medium (CellGenix Inc.) with thrombopoietin, stem cell factor, and FLT3 ligand (PeproTech) all at 100 ng/ml]. Either IL-3 (60 ng/ml) or IL-6 (100 ng/ml) was added to the media as noted. The small molecules StemRegenin 1 (STEMCELL Technologies), UM171 (ApexBio), and eltrombopag (Selleckchem) were added to the stem cell growth media used throughout entire experiment at 1 μM, 35 nM, and 3 μg/ml respectively.

Gene editing CD34⁺ hematopoietic stem/progenitor cells. CD34⁺ cells were pre-stimulated in stem cell growth media with cytokine for 48 hours at 37° C., then electroporated with the Neon Transfection System and 10-μl tip (Thermo Fisher Scientific). Cells were dispensed into a 24 well plate containing 400 μL of media with donor template AAV at MOI between 1000 and 5000. Twenty-four hours after electroporation and AAV transduction, AAV containing media was removed and replaced with fresh stem cell growth media. Analysis of viability and GFP was performed at days 2 and 5 after editing. Analysis of stem cell phenotype (CD34⁺CD38⁻CD90⁺CD133⁺) was performed at 24 or 48 hours after editing. When scaling up for murine engraftment experiments, the same editing conditions were used except for the following changes: 100 μL Neon tips were used for electroporations, with identical concentrations of cells and nuclease. Cells were dispensed into 4 mL of media containing AAV in a 6 well plate. Twenty-four hours later, cells were collected and washed twice with PBS prior to injection.

AAV stocks were produced as previously described (Khan, I. F., R. K. Hirata, and D. W. Russell, Nat Protoc, 2011. 6(4): p. 482-501). The AAV vector, serotype helper, and adenoviral helper (HgT1-adeno) plasmids were transfected in to HEK293T cells. Cells were collected 48 hours later and lysed by 3 freeze-thaw cycles. The lysate was subsequently treated with benzonase and purified over iodixanol density gradient. Titers of the viral stocks were determined by qPCR of AAV genomes using inverted terminal repeat (ITR)-specific primers and probe (Aurnhammer, C., et al., Hum Gene Ther Methods, 2012. 23(1): p. 18-28).

Recombinant AAV vectors. All plasmids were adapted from those described previously in Hubbard et al. (Hubbard, N., et al., Blood, 2016. 127(21): p. 2513-22). To generate the pAAV.MND.GFP.WPRE reporter construct, the MND modified retroviral promoter was inserted into a previously described pAAV CD40LG[GFP.WPRE] plasmid (SEQ ID NO: 16).

A pAAV.CD40L.cDNA.WPRE3 (SEQ ID NO: 15) donor template was designed with 1 kb regions of homology flanking a codon-optimized CD40L, truncated Woodchuck Hepatitis Virus Post-transcriptional Regulatory Element 3 (WPRE3), and late SV40 polyadenylation signal containing an additional upstream sequence element (Choi, J.-H., et al., Molecular brain, 2014. 7(1): p. 17). The full CD40L.cDNA.WPRE3.synPolyA was gene synthesized (GeneArt), and cloned into a pAAV backbone with two flanking 1 kb regions of homology to the CD40LG locus by Infusion cloning (Clontech).

Statistical analyses were performed using GraphPad Prism 7 (GraphPad, San Diego, Calif.). For multiple comparisons, p-values were calculated using one-way ANOVA. Unless otherwise stated, for comparing two groups, p values were calculated using un-paired two-tailed t test. All bar graphs show mean±SEM.

All electroporations were performed using the Neon Transfection System (Thermo Scientific). Prior to electroporation, cells were collected and washed with PBS. Cells are resuspended in Neon Buffer T such that, after addition of Cas9 RNP (100 or 200 μg of Cas9 protein/ml of total reaction volume) or mRNA (50 μg each CD40L TALEN/ml of total reaction volume), the final concentration is 2.5×10⁷ cells/ml. After mixing with nuclease, 2.5×10⁵ cells are electroporated (1400 V, 20 ms, one pulse) per 10 μL Neon tip.

When scaling up for mouse engraftment studies, all reagent concentrations were identical but total amounts were increased ten-fold per electroporation, and 100 μL Neon tips are used. Thus, 2.5×10⁶ cells are mixed with Cas9 RNP (100 or 200 μg/ml) or mRNA (50 μg/ml each CD40L TALEN) and electroporated using a 100 μL Neon tip. Multiple 100 reactions are performed for each condition.

Colony forming units. Mock or gene-edited HSPCs were mixed vigorously with methylcellulose-based MethoCult H4034 Optimum medium (STEMCELL Technologies) by vortexing. After mixing, 500 cells in 1.1 ml of methylcellulose-based media were dispensed into a 35 mm gridded dish. Cells were incubated for 14 days at 37° C. and colonies were categorized and quantified based upon manufacture documentation (STEMCELL Technologies). GFP expressing colonies were quantified using an EVOS fl inverted microscope (AMG).

Genomic DNA was isolated from hematopoietic stem and progenitor cells (HSPCs) using a DNeasy Blood and Tissue kit (Qiagen). To assess editing rates at the CD40LG locus, “in-out” droplet digital PCR was performed with the forward primer binding within the AAV insert and the reverse primer binding the CD40LG locus outside the region of homology. A control amplicon of similar size (1.3 kb) was generated for the ActB gene (SEQ ID NO: 17) to serve as a control. Probes for both amplicons were labeled with FAM and the reactions were performed in separate wells. Two duplicates were performed in separate wells for each HDR and control reaction. The PCR reactions were partitioned into droplets using a QX200 Droplet Generator (Bio-Rad). Amplification was performed using ddPCR Supermix for Probes without UTP (Bio-Rad), 900 nM of primers, 250 nM of Probe, 50 ng of genomic DNA, and 1% DMSO. Droplets were analyzed using the QX200 Droplet Digital PCR System (Bio-Rad) and analyzed using QuantaSoft software (Bio-Rad). As CD40LG resides on the X chromosome, the editing rates from male donor cells were calculated as a ratio of copies/μ1 from CD40LG/ActB positive droplets multiplied by 2.

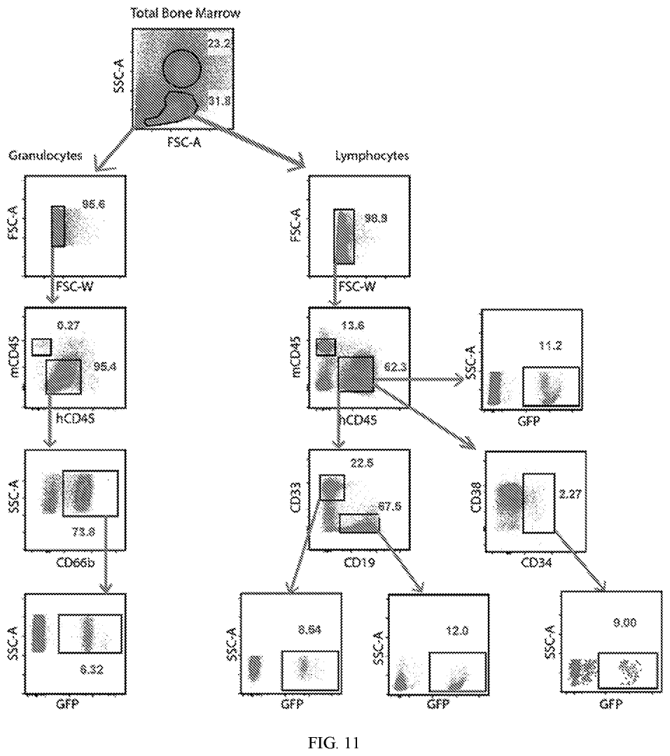

Flow cytometric analysis was done on an LSR II flow cytometer (BD Biosciences) and data was analyzed using FlowJo software (Tree Star). To assess engraftment of edited cells in various hematopoietic lineages within the bone marrow and spleen, cells were stained with the following fluorophore-conjugated antibodies: human CD45-eFluor450 (clone HI30, eBioscience), mouse CD45-APC (clone 30-F11, eBioscience), CD33-PE (clone WM53, BD Biosciences), and CD19-Pe/Cy7 (clone HIB19, eBioscience) (see FIG. 11 for detailed gating strategy). To assess hematopoietic stem cell phenotype, cells were stained with the following fluorophore-conjugated antibodies: CD34-APCCy7 (clone 581, BioLegend), CD38-PerCPCy5.5 (clone HIT2, BD Biosciences), CD90-APC (clone 5E10, BD Biosciences), and CD133-PE (clone AC133, Miltenyi Biotec).

mRNA synthesis. pEVL CD40LG.TALEN (SEQ ID NOs: 21 and 22) constructs were linearized using Bsal. In vitro transcription and 5′-capping were performed using the T7 mScript Standard mRNA production System (Cellscript) according to manufacturer's protocols. Linearized template was in vitro transcribed into unmodified mRNA transcripts and capped (5′-7-methylguanylate cap) with cap-1 mRNA structure (2′-O-methyltransferase) using provided enzymes. Final purification was performed using NucleoSpin RNA Clean-up kit (Machery Nagel).

Nuclease design. CD40LG TALENs were identical to those previously described in Hubbard et al., except that they were cloned into a pEVL backbone (SEQ ID NO: 20) rather than a pUC57 backbone (Hubbard, N., et al., Blood, 2016. 127(21): p. 2513-22; Grier, A. E., et al., Molecular Therapy-Nucleic Acids, 2016. 5).

CRISPR sgRNA (SEQ ID NO: 12) was designed to generate a double-stranded break (DSB) as close to as possible to the DSB site generated by the CD40LG TALEN pair so as not to bias the comparison of the two nuclease platforms. The synthetic sgRNA (5′-AAAGUUGAAAUGGUAUCUUC-3′, SEQ ID NO:28) was purchased from Synthego (Pleasanton, Calif.), and was chemically modified with 2′-O-methyl analogs and 3′ phosphorothioate internucleotide linkages between the three terminal nucleotides both the 5′ and 3′ ends. Cas9 RNP was made by incubating Cas9 protein (Integrated DNA Technologies) with sgRNA at a 1:1.2 molar ratio for 15 minutes immediately prior to electroporation.

### Example 1—CD40LG-Targeted HDR in CD34+ Hematopoietic Stem Cells

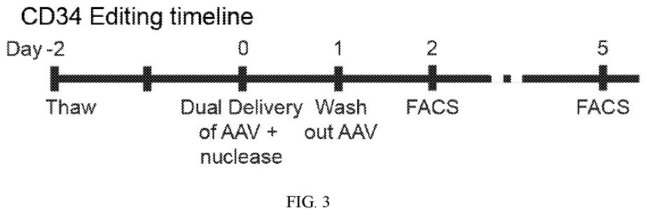

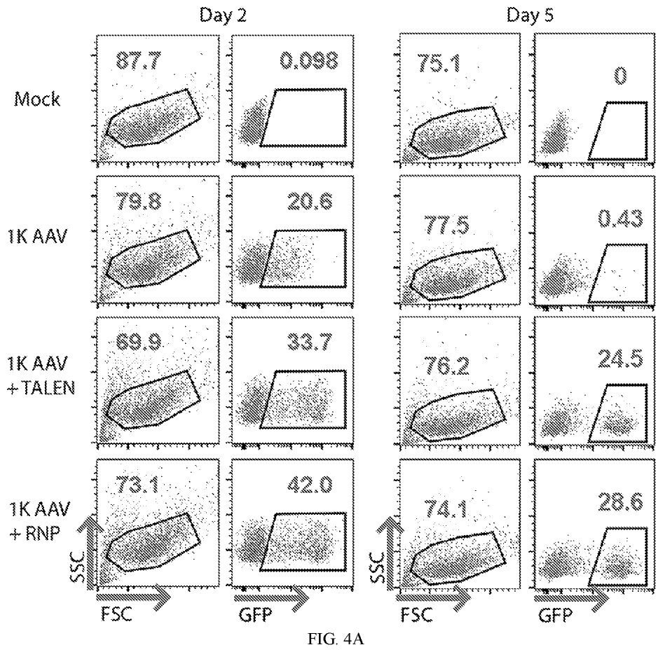

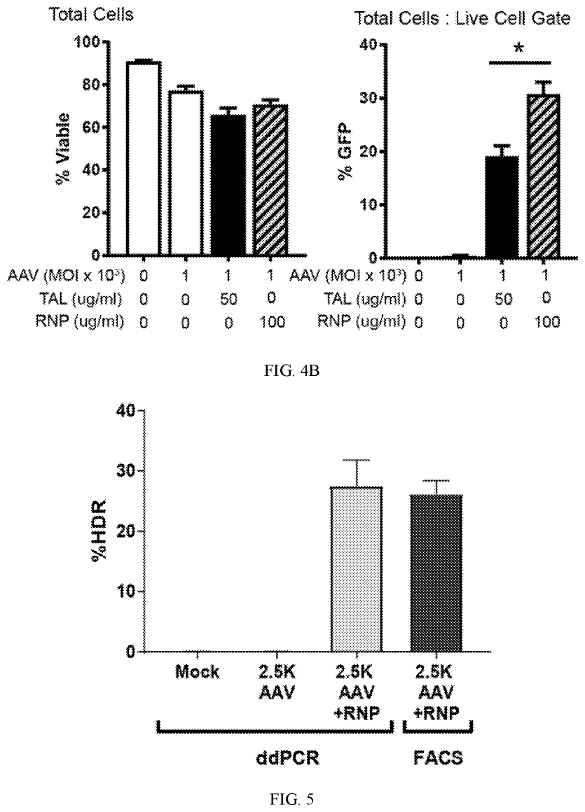

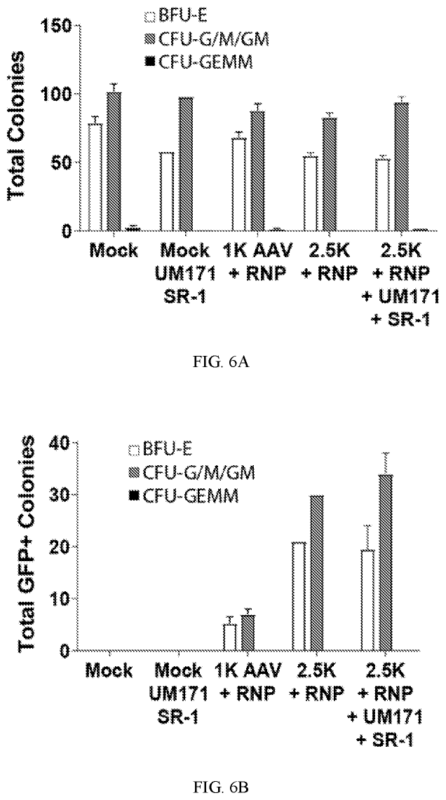

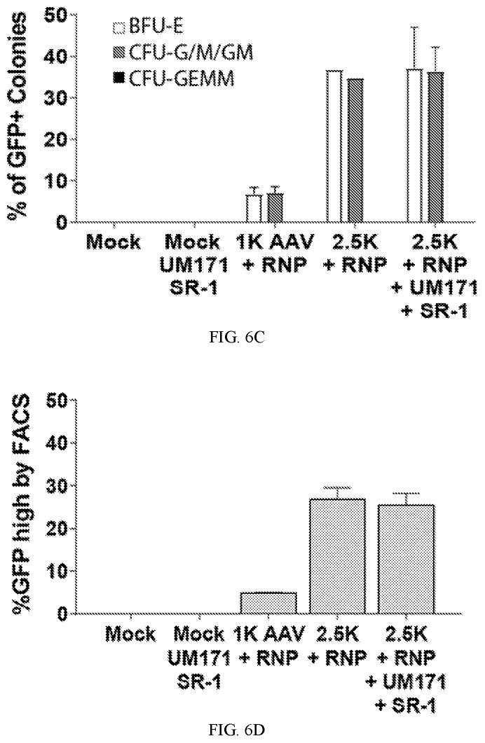

This example demonstrates methods for introducing a GFP reporter at the CD40LG locus in CD34+ hematopoietic stem cells. Aspects of the systems, methods, and compositions described herein relate to improving the efficiency for editing blood mobilized CD34⁺ cells enriched from healthy donors, including cytokine pre-stimulatory conditions, optimal cell density, electroporation conditions, and the relative timing of AAV and nuclease delivery (FIG. 3). Frozen CD34⁺ cells were thawed and cultured for 48 hours, before dual delivery of nuclease and AAV. Two days after dual delivery of AAV6 donor template and either TALEN or RNP nuclease, cells were isolated and delivery was analyzed (FIG. 3). Cells showed only 20% reduction in viability compared to mock treated cells (FIG. 4A). Five days after gene editing, background GFP expression in the AAV only control, representing expression from non-integrated AAV, was reduced to nearly zero (FIG. 4A). At this same time, about 20% of AAV plus TALEN (AAV/TALEN) treated cells, and about 30% of AAV/RNP treated cells expressed GFP at a high MFI, indicative of HDR (FIG. 4B). Seamless on-target integration of the MND. GFP reporter template was confirmed by droplet-digital PCR (ddPCR) of cells treated with AAV/RNP (FIG. 5). In addition, cells treated with AAV/RNP did not differ in total colony number or colony type compared to mock treated cells (FIG. 6A and FIG. 6B) as determined by methylcellulose colony forming unit (CFU) assay. Furthermore, the percentage of HDR-edited colonies is not skewed by colony type, and is similar to the percentage of GFP⁺ colonies detected by flow cytometry (FIG. 6C, FIG. 6C, FIG. 6D). Due to increased viability and rates of HDR by RNP compared to TALEN, all subsequent experiments were performed using RNP nuclease.

### Example 2—Cytokine Conditions for HDR in Hematopoietic Stem Cells

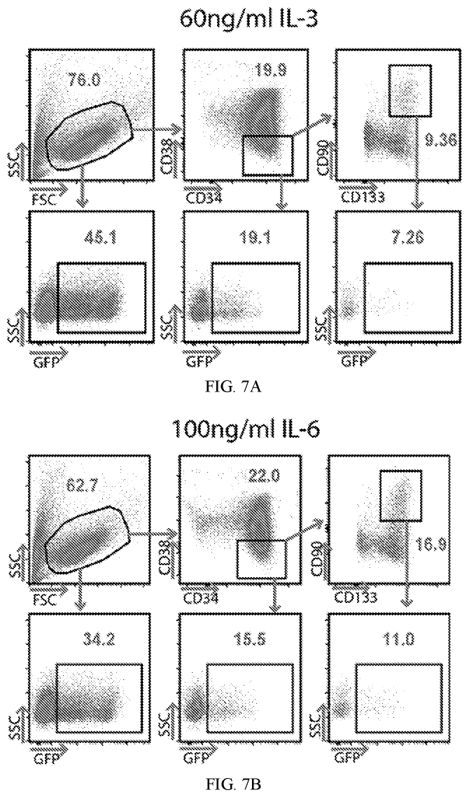

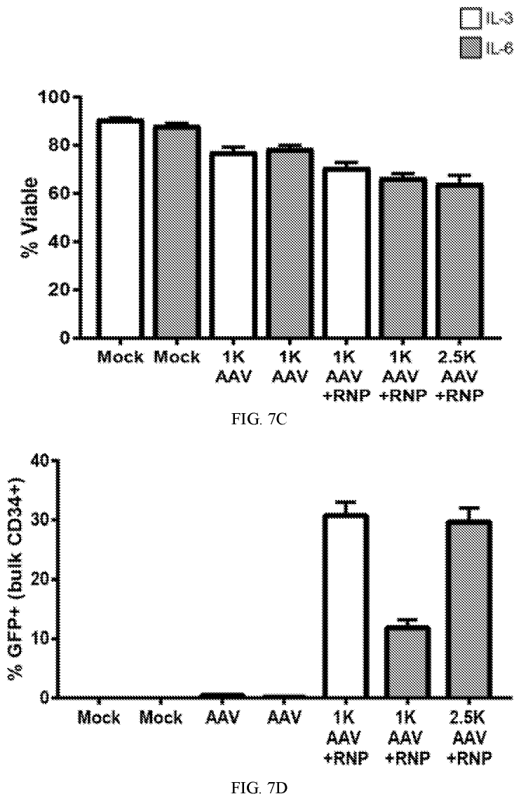

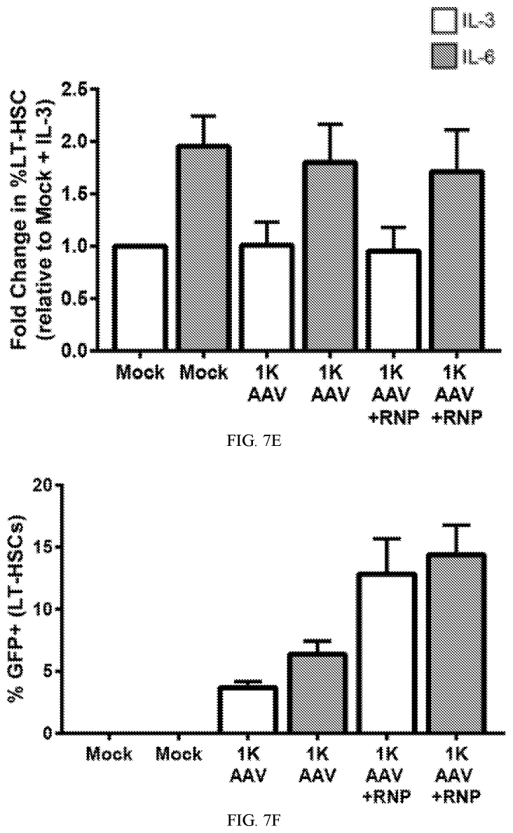

This example demonstrates methods for modulating cytokine response for HDR in hematopoietic stem cells. Two cytokine cocktails for use in ex vivo culturing for CD34⁺ cells were tested. Both cocktails included 100 ng/ml of stem cell factor (SCF), FMS-like tyrosine kinase-3 (Flt-3), and thrombopoietin (TPO), to which either 60 ng/ml of interleukin-3 (IL-3) or 100 ng/ml of interleukin-6 (IL-6) were added to promote cell expansion. Cells grown in media containing IL-3 exhibited higher viability 48 hours after editing (FIG. 7A, FIG. 7B, FIG. 7C, FIG. 7D), and higher rates of HDR in the bulk population (FIG. 7E). However, when cultured with IL-6, the LT-HSC population (CD34⁺, CD38⁻, CD90⁺, and CD133⁺) recovered at twice the number of IL-3-treated cells. In addition, a slightly higher percentage of cells cultured in the presence of IL-6 expressed GFP within the LT-HSC population compared with IL-3. Additionally, robust rates of HDR were achieved in the bulk cell population with IL-6 by increasing the AAV dose to 2500 viral particles per cell and RNP dose to 200 μg/ml.

### Example 3—Insertion of CD40L cDNA Regulated by the Endogenous Promoter

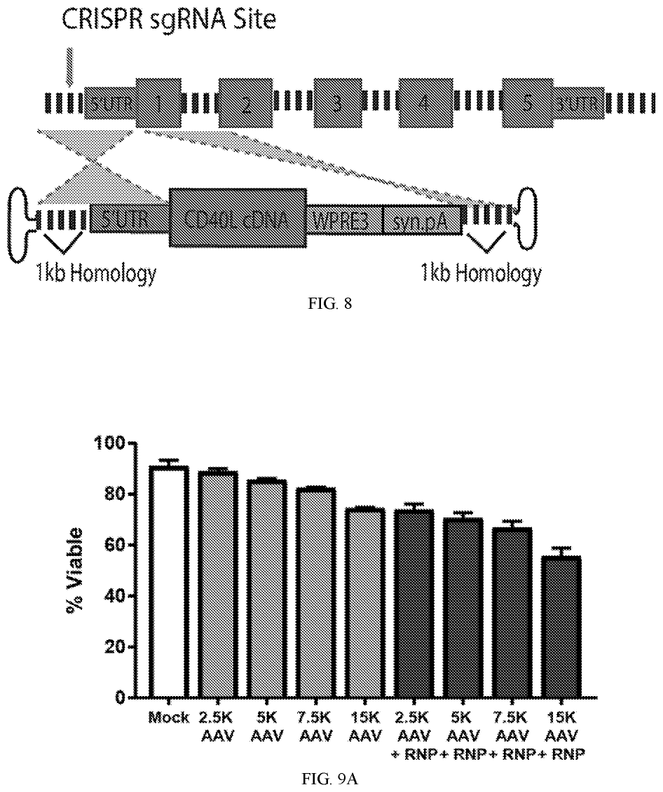

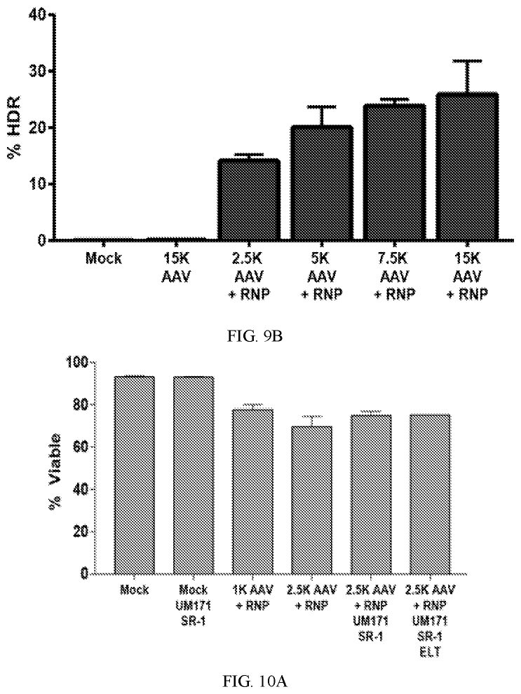

This example demonstrates methods for efficient incorporation of CD40L cDNA downstream of endogenous CD40LG promoter. This example demonstrates endogenous promoter-driven expression of CD40L cDNA for therapeutic purposes. T cells edited with CD40L cDNA introduced downstream of the endogenous promoter demonstrate equivalent surface expression of CD40L as non-edited cells (Hubbard, N., et al., Blood, 2016. 127(21): p. 2513-22). To edit hematopoietic stem cells, an AAV donor template (SEQ ID NO: 15) containing identical 1 kb homology arms flanking codon-optimized CD40L cDNA, a WPRE3 element, and a synthetic polyadenylation sequence was designed (FIG. 8) (Choi, J.-H., et al., Molecular brain, 2014. 7(1): p. 17). Targeted integration of the cDNA was observed in about 25% of the cells as measured by ddPCR (FIG. 9A and FIG. 9B). Thus, CD40L cDNA was successfully introduced downstream of the endogenous CD40LG promoter.

### Example 4—Engraftment of Edited Cells in NOD-Scid-IL2Rg^{NULL} Mice

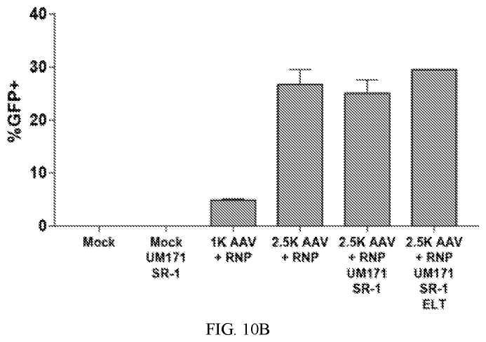

This example demonstrates methods for engraftment of edited cells in the bone marrow of NSG mice. Following robust editing of CD34⁺ cells, the cells were transplanted into NOD-scid-IL2Rg^{NULL} (NSG) mice to determine the long-term repopulation potential of edited cells. The MND.GFP reporter construct was used to edit these cells in order to easily track, and determine the phenotype of edited cells. To achieve this, editing reagents were used at either a “high dose” (2.5K MOI AAV and 200 μg/ml RNP) or “low dose” (1K MOI AAV and 100 μg/ml RNP). When scaled up for in vivo experiments, comparable rates of HDR with increased viability post editing compared to smaller scale studies (FIG. 10A and FIG. 10B). To reduce culturing time, edited cells were transplanted into NSG mice 24 hours after delivery of AAV and RNP. Bone marrow and spleen from each animal was harvested 12-16 weeks after transplantation and analyzed for the presence of HDR-edited human cells (FIG. 11).

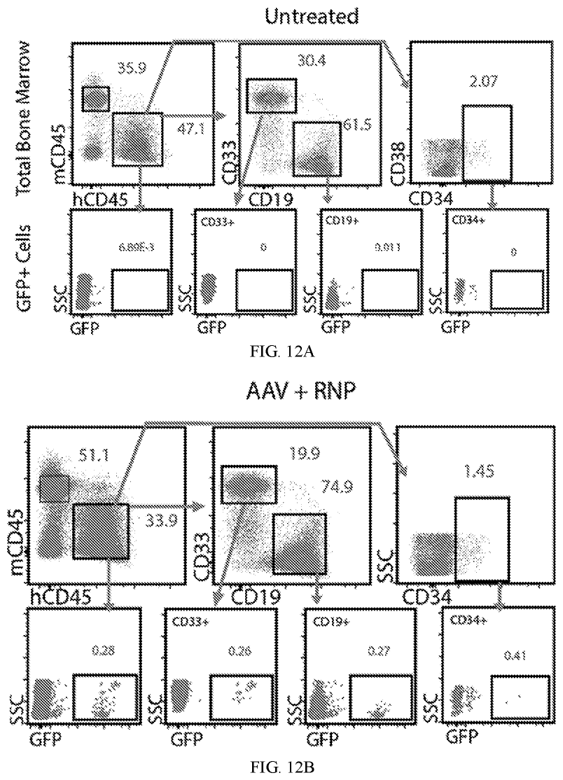

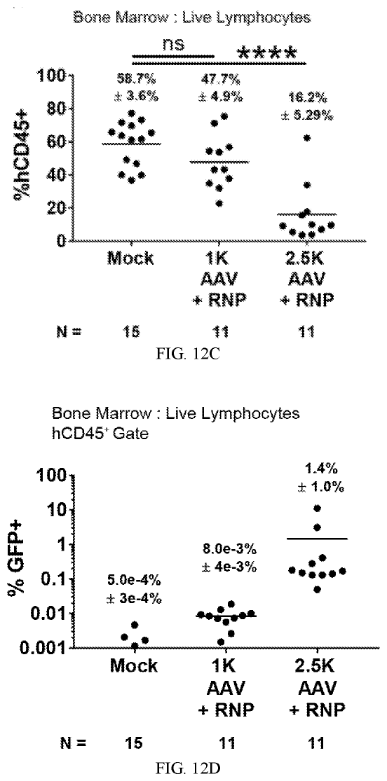

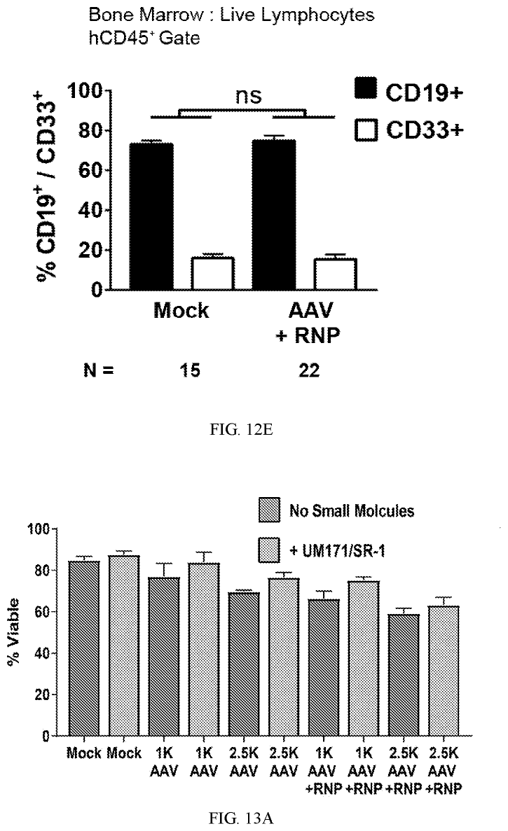

Cells treated with 1K MOI AAV and 100 μg/ml RNP had an average hCD45 engraftment of 47.7% in the bone marrow compared to 59% in untreated cells (FIG. 12C). On the other hand, cells treated with 2.5K MOI AAV and 200 μg/ml RNP showed a more pronounced reduction in hCD45 engraftment (16.2%) compared to untreated CD34⁺ cells (59%). GFP expression was detected in 1.4% of the human cells edited with high dose AAV plus RNP, indicating that mice received HDR-edited long-term repopulating hematopoietic stem cells. In comparison, only 5.0e-3% of the cells treated with low dose AAV plus RNP expressed GFP upon recovery from mice (FIG. 12D). This nearly 3-log difference in percentage of edited cells persisting in the bone marrow was striking, given the difference in HDR rates between the two cell populations at the time of transplantation was 26.8% for high dose versus 4.9% for the low dose-treated cells (FIG. 10A and FIG. 10B).

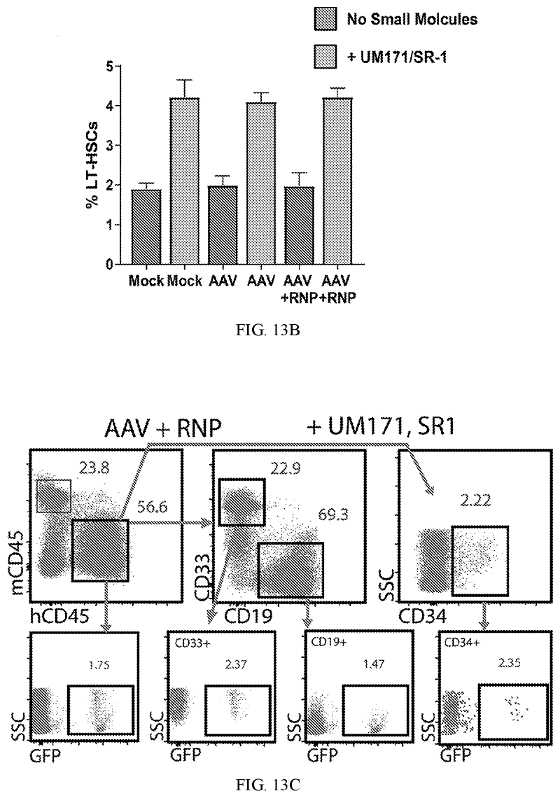

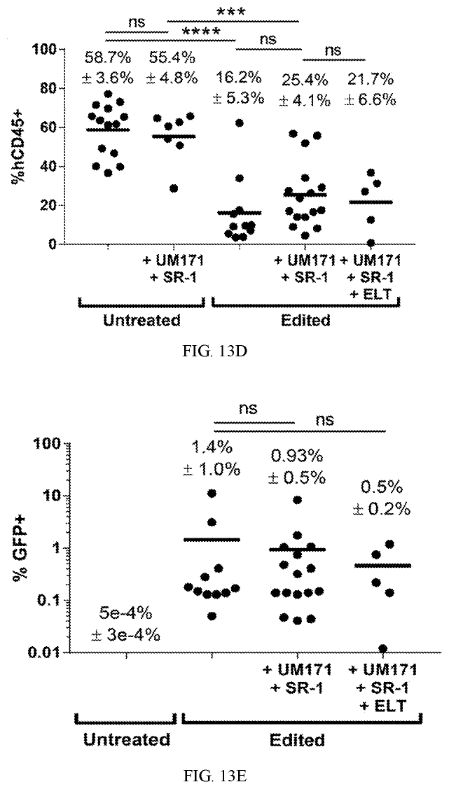

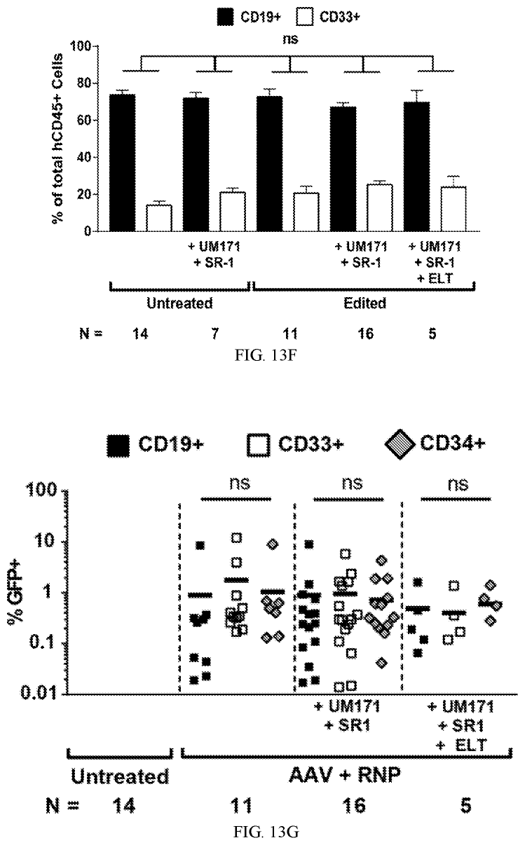

The phenotype of cells treated with AAV plus RNP was similar to untreated control cells. In mice receiving either AAV plus RNP treated cells or untreated cells, the proportion of CD33⁺, CD19⁺, and CD34⁺ was not significantly different (FIG. 12E). Additionally, edited GFP⁺ cells were found in each of the CD33⁺, CD19⁺, and CD34⁺ populations at similar rates (FIG. 12A, FIG. 12B, and FIG. 13G). This suggested that editing did not disrupt the differentiation potential of hematopoietic cells, and that true LT-HSCs were being edited.

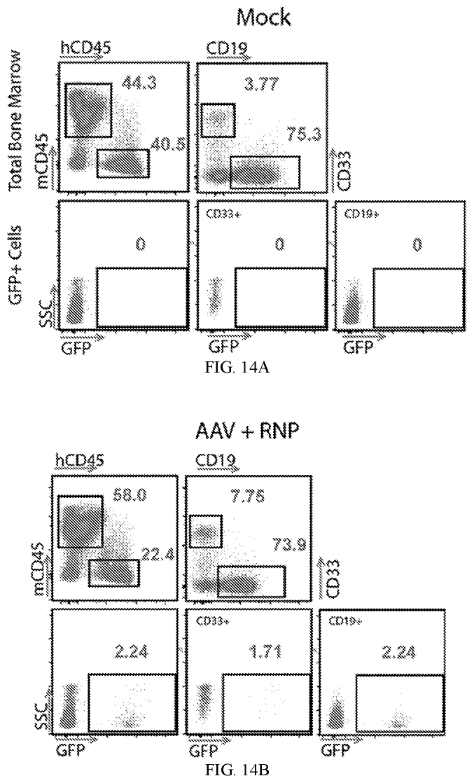

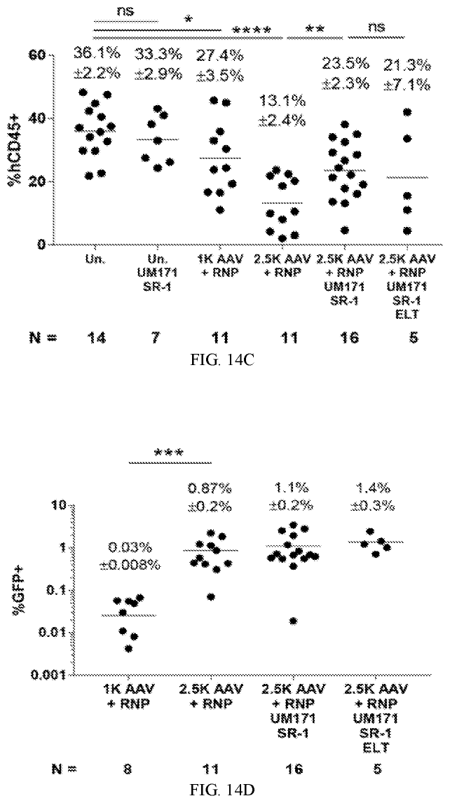

Similar patterns of hCD45 engraftment and GFP expression were found in the spleen. Mice transplanted with low-dose edited cells showed minimal reduction in the percentage of splenic hCD45⁺ cells (27.4% vs. 36%) compared to mice receiving untreated cells (FIG. 14A, FIG. 14B, FIG. 14C, FIG. 14D). The spleens of mice transplanted with high-dose edited cells consisted of 13.1% hCD45⁺ cells. The GFP-expressing fraction of hCD45⁺ cells was 0.9% in mice receiving high dose edited cells, while mice receiving low dose edited cells only had 0.03% GFP positivity among hCD45⁺ cells (FIG. 14A, FIG. 14B, FIG. 14C, FIG. 14D).

### Example 5—Effect of Small Molecules on the Engraftment of Edited Cells

This example demonstrates the impact of small molecules on engraftment of edited cells in NSG mice. Edited cells were detected in NSG mice up to 16 weeks post transplantation. This example demonstrates methods to further increase the number of LT-HSCs that are edited and able to engraft. Small molecules that promote the self-renewal of LT-HSCs ex vivo were introduced into the culture system (Fares, I., et al., Science, 2014. 345(6203): p. 1509-12; Sun, H., et al., Stem Cell Res, 2012. 9(2): p. 77-86). Inducing self-renewal provided a two-fold benefit: it was useful for cells to be cycling (in G or S2 phase) in order to undergo HDR, and self-renewing stem cells maintained their ability to engraft in the bone marrow and persist long term.

The small molecules UM171 and SR-1 increase the self-renewal of hematopoietic stem cells, and the combination has recently been used to support engraftment of HDR-edited cells (Schiroli, G., et al., Sci Transl Med, 2017. 9(411); Bak, R. O. and M. H. Porteus, Cell Rep, 2017. 20(3): p. 750-756; Bak, R. O., et al., Elife, 2017, 6; Fares I., et al., (2013) Blood 122:798). The effect of these small molecules on editing rates and engraftment of edited cells was analyzed. Adding UM171 and SR-1 to the culture media slightly increased cell viability following editing, but did not have a significant effect on the rate of HDR in the bulk population, or in cells that stained as LT-HSCs (FIG. 13A and FIG. 15A, FIG. 15B, FIG. 15C, FIG. 15D, FIG. 15E). Notably, the percentage of cells staining as LT-HSCs 48 hours after editing more than doubled with the addition of the small molecules (FIG. 13B). Collectively, these data provided evidence that in some alternatives of the culture system described herein, addition of UM171 and SR-1 maintained the stemness of HSCs more so than cytokines alone, but they did not impact the rates of HDR.

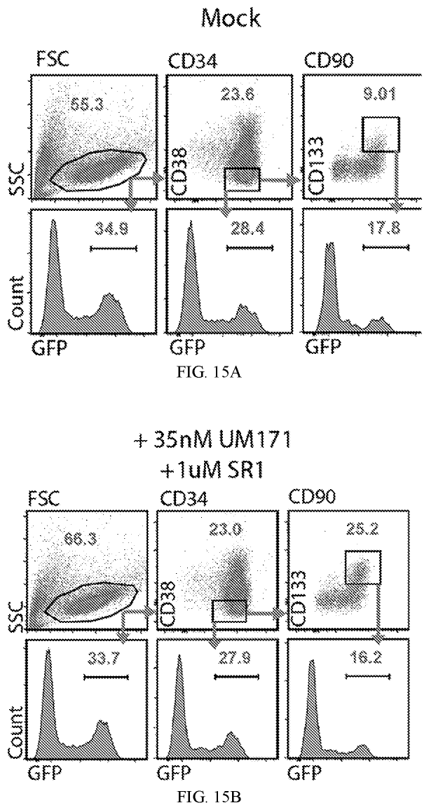

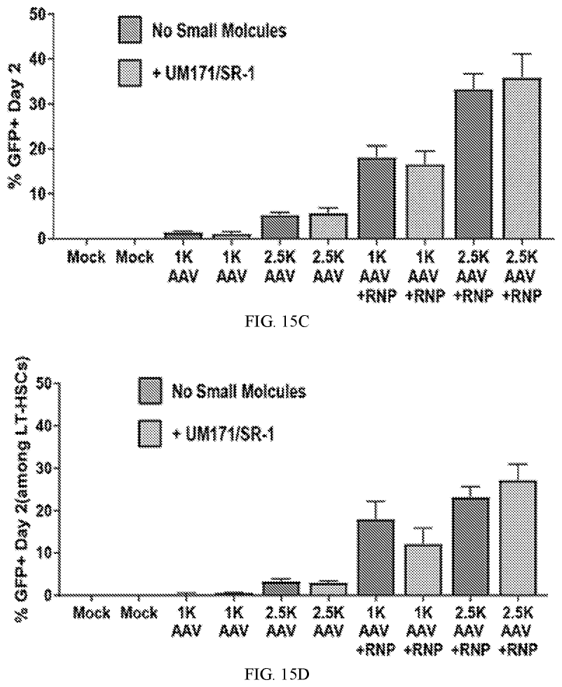

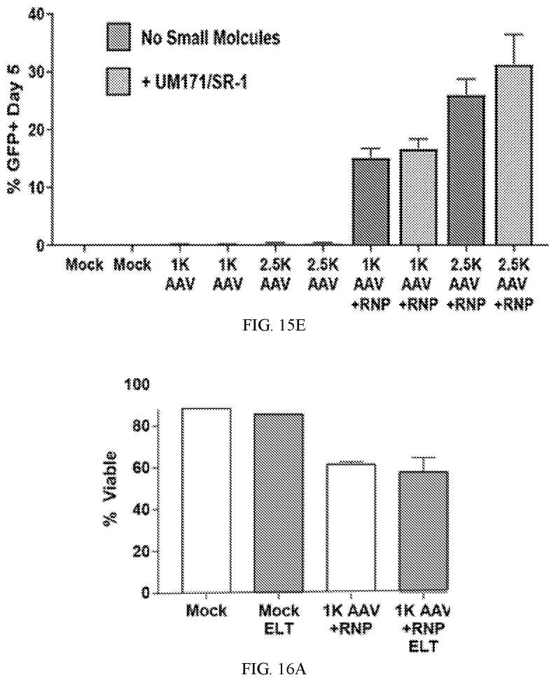

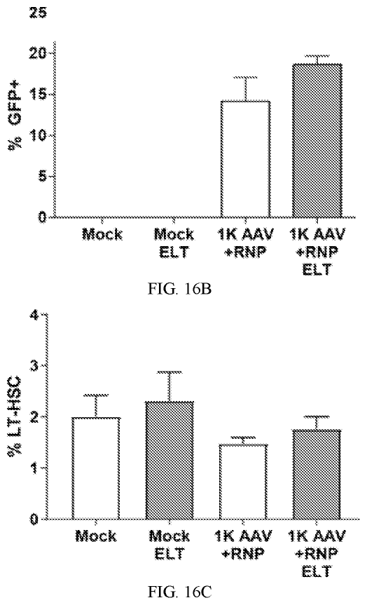

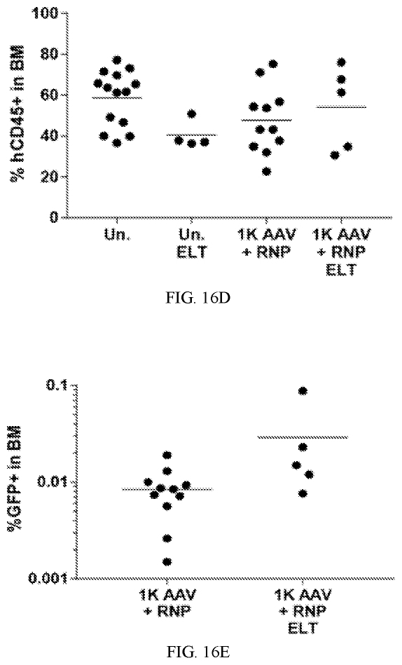

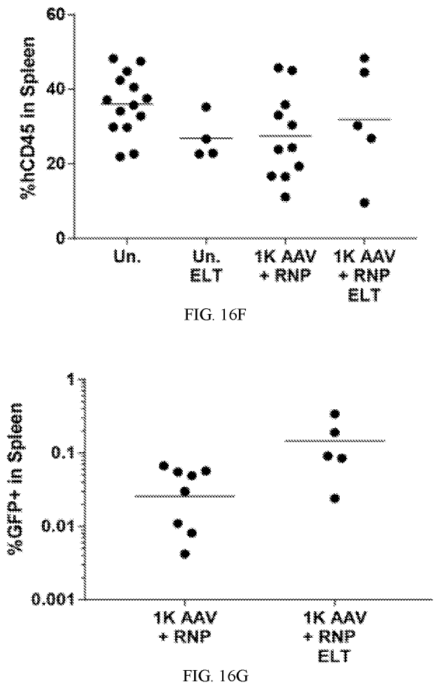

Another small molecule, eltrombopag, can expand CD34⁺/CD38⁻ cells (Sun, H., et al., Stem Cell Res, 2012. 9(2): p. 77-86). Eltrombopag, an approved drug for the treatment of aplastic anemia, is an agonist of the TPO receptor, c-mpl. Adding eltrombopag increased the percentage of cells that are edited by about 5%, without significantly impacting viability (FIG. 16A, FIG. 16B, FIG. 16C, FIG. 16D, FIG. 16E, FIG. 16F, FIG. 16G). There was a slight increase in the percentage of cells that stained as LT-HSCs with the drug.

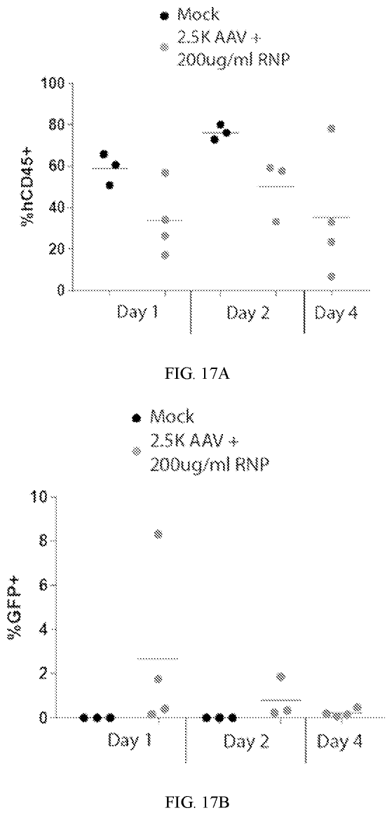

The impact of these small molecules on the ability of edited cells to engraft in NSG mice was tested. The addition of UM171 and SR-1 slightly increased overall hCD45 engraftment in the bone marrow for cells that were treated with AAV plus RNP, from 16.2% to 25.4% (FIG. 13D). The percentage of human cells that were GFP⁺ was similar with or without with the two small molecules (FIG. 13E). The percentage of CD33⁺ myeloid and CD19⁺ B cells among the human cells in the bone marrow was also not significantly different, and edited cells were detected in both populations at roughly equivalent rates (FIG. 13F and FIG. 13G). Additionally, in the presence of UM171 and SR-1, the percentage of edited cells in the bone marrow was highest when cells were transplanted 1 day after editing, rather than 2 days or 4 days (FIG. 17A, FIG. 17B).

When eltrombopag was added to culture media, there was no impact on overall hCD45 engraftment in the bone marrow or spleen (FIG. 16A, FIG. 16B, FIG. 16C, FIG. 16D, FIG. 16E, FIG. 16F, FIG. 16G). However, with low dose AAV+RNP, there was a small increase in the percentage of GFP⁺ cells in both bone marrow and spleen. In contrast, when high dose AAV plus RNP were used along with UM171 and SR1, there was no increase in the percentage of edited cells with eltrombopag (FIG. 13E). It was possible that an increase in HDR rates due to eltrombopag was only detectable in vivo when the baseline HDR rates were low. Collectively, these data provided evidence that eltrombopag did not significantly impact the engraftment of hCD45⁺ cells or the editing rate among LT-HSCs. The small molecule combination of UM171 and SR1 did not increase the rates of HDR in hematopoietic stem cells, but did increase their overall rates of hCD45⁺ engraftment when transplanted into NSG mice.

### Example 6—Effect of Pre-Stimulation Time on In Vivo Engraftment Potential

The effect of a longer pre-stimulation time on the in vivo engraftment potential of HDR edited mobilized human CD34+ cells was tested. Adult mobilized human CD34⁺ cells were cultured at a concentration of 1×10⁶ cells/ml in SCGM media supplemented with TPO, SCF, FLT3L and IL6 (100 ng/ml) plus 35 nM UM171, 1 μM SR1 for either 48 hours or 72 hours. Cultured cells were then electroporated of 200 μg/ml of RNP using a Neon system, then transduced with rAAV6 targeting vector containing a GFP reporter cassette at the MOI of 1K. After 24 hours, transduced cells were transplanted into NSGW41 recipient mice. Mice were injected with 12.5 mg/kg Busulfan one day prior to transplantation and sacrificed at 16 weeks post transplantation.

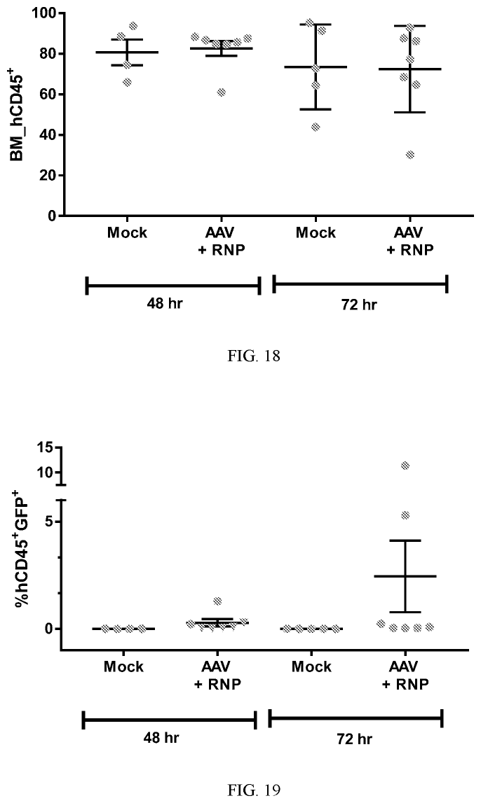

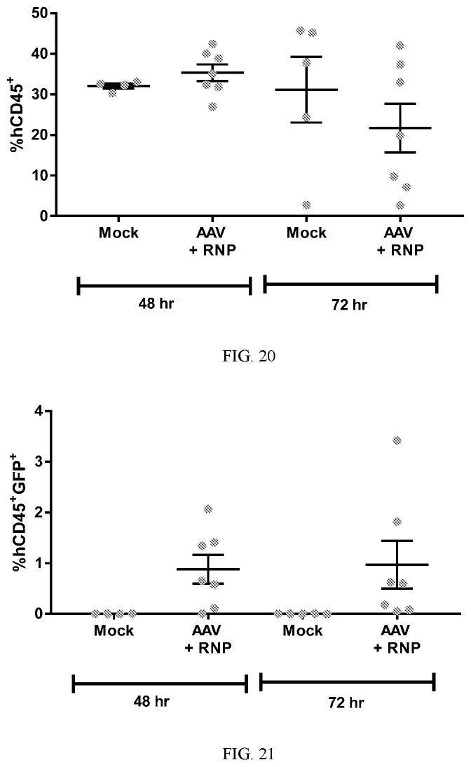

Total engraftment of human cells was relatively lower in CD34+ cells pre-stimulated with cytokines for 48 hours. compared to cells pre-stimulated for 72 hours in both bone marrow and spleen (FIGS. 18 and 20). However, engraftment of edited (GFP+) cells was relatively higher with the longer pre-stimulation (FIG. 19 and FIG. 21).

### Example 7—Comparison of Culture Protocols

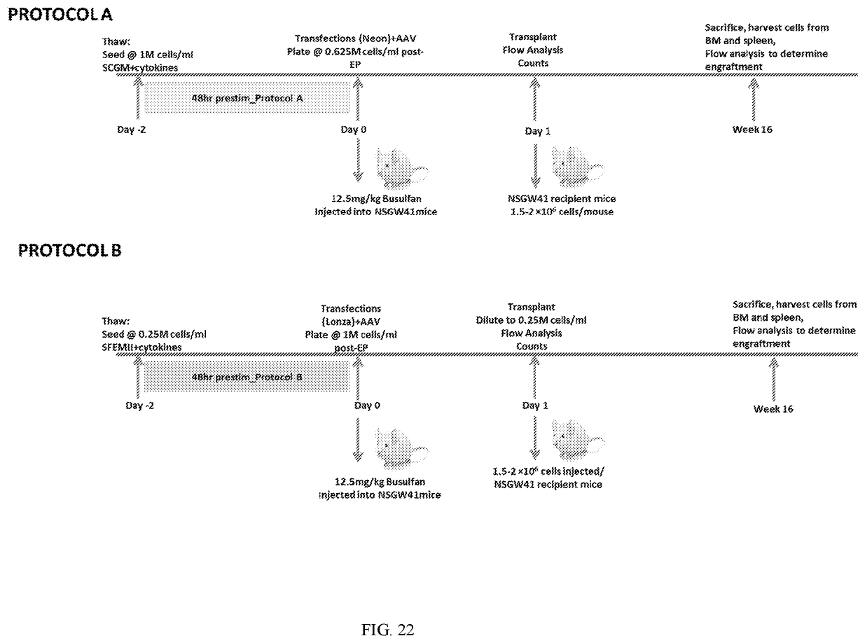

Two alternative protocols (Protocols A or B) for culturing CD34⁺ cells were examined. TABLE 1 and FIG. 22 outline conditions for each protocol for culturing mobilized CD34⁺ HSCs for HDR-based gene editing.

TABLE 1

| Parameter | Protocol A | Protocol B |
|---|---|---|
| Media | SCGM | SFEMII |
| Human cytokines | TPO, FLT3, SCF, IL-6 | TPO, FLT3, SCF, IL-6 |
| | (100 ng/ml) | (100 ng/ml) |
| Compounds | UM171 and SR1 | UM171 and SR1 |
| Pre-stimulation: cell | 1.00E+06 | 2.50E+05 |
| concentration/ml | | |
| Pre-stimulation time | 48 hours | 48 hours |
| RNP dose | 1 μg (1.2:1 molar | 1 μg (1.2:1 molar |
| | ratio) | ratio) |
| Cell concentration | 1 million/20 μl rxn | 1 million/20 μl rxn |
| during EP | with Lonza (CM149 | with Lonza (CM149 |
| | program) | program) |
| AAV MOI | 1K | 1K, 2.5K |
| Cell concentration for | 0.5 million/0.8 ml | 1 million/1 ml |
| transduction | | |
| 16 hours after | add media (dilute virus) | cells diluted to 0.25 |
| transduction | | million cell/ml |

For protocol A, mobilized human CD34+ cells were cultured in SCGM media supplemented with TPO, SCF, FLT3L and IL6 (100 ng/ml) plus 35 nM UM171, 1 SR1 for 48 hours at a concentration of 1×10⁶ cells/ml, followed by nucleofection of 200 μg/ml of RNP using a Lonza system. The cells were subsequently transduced with AAV targeting vector at the MOI of 1K. For protocol B, CD34+ cells were cultured in SFEMII media containing the same supplements as above. The cell density during pre-stimulation was 2.50E+05/ml. Following the 48 hour pre-stimulation, the cells were nucleofected with 200 μg/ml of RNP with the Lonza system and plated at a density of 1×10⁶ cells/ml prior to transduction with AAV at the MOI of 2.5K. The cells were transplanted into W41 mice the following day. Mice were injected with 12.5 mg/kg Busulfan one day prior to transplantation of cells.

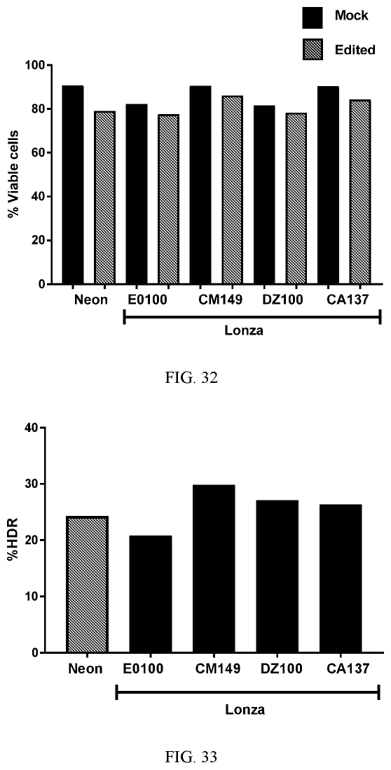

In Examples 1-5, protocol A was used, and electroporation was performed using the Neon electroporation system. A comparison of cell viability was performed using various nucleofection programs on the Lonza system, compared to electroporation by Neon system (FIG. 32). Adult mobilized human CD34⁺ cells were cultured in SCGM media followed by mock transfection or transfection of RNPs (200 μg/ml) by Neon or Lonza instruments. A comparison of percent HDR (GFP expression) using various nucleofection programs on the Lonza system versus electroporation by the Neon system was also performed (FIG. 33). Adult mobilized human CD34⁺ cells were cultured in SCGM media followed by RNP (200 μg/ml) electroporation using Neon or nucleofection by Lonza. Electroporation was followed by transduction with AAV targeting vector. HDR rates were determined by GFP expression on day 5. CM149 lead to optimal viability and HDR rates and was therefore used for optimized in vivo studies. Conditions were identified that had increased CD34+ cell viability and HDR rates using the Lonza system and electroporation program CM149 (FIG. 32 and FIG. 33). The increase in the average number of long-term engrafted, HDR-edited cells GFP+ cells likely reflected use of Lonza nucleofector system for the nuclease delivery.

In the following, CD34+ cells were cultured using either protocol A or B and were transfected using the Lonza nucleofection system.

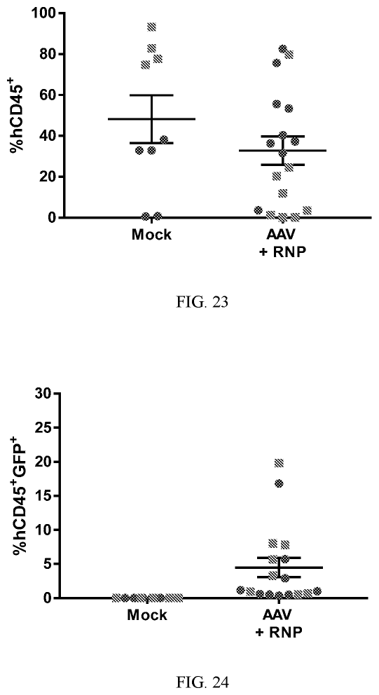

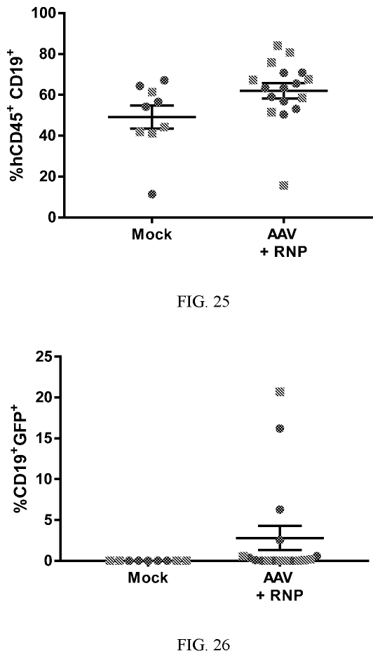

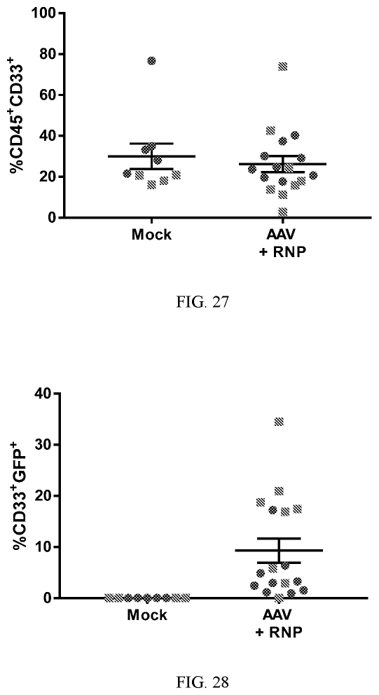

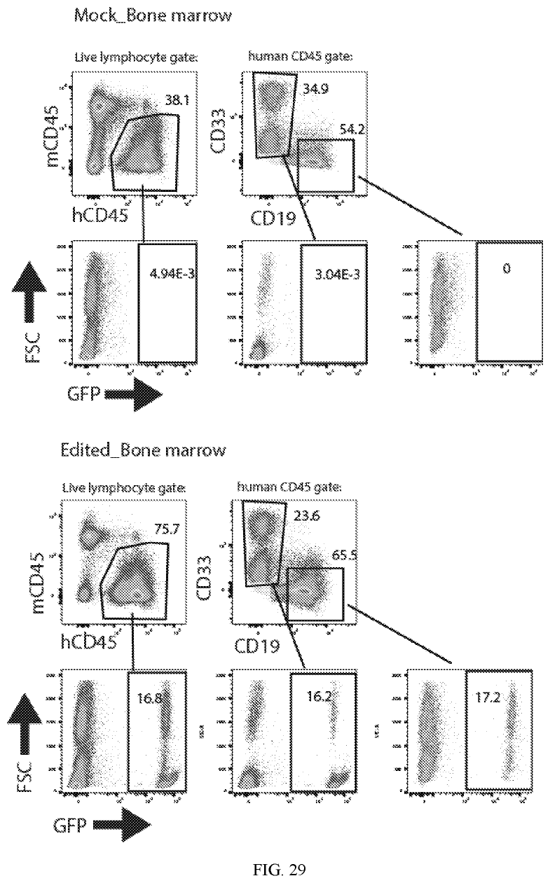

With regard to cells recovered from bone marrow, the percent hCD45⁺ cells recovered from the bone marrow of NSGW41 mice transplanted with CD34⁺ cells cultured using protocol A or B was determined (FIG. 23), and the percent GFP+ among the total hCD45+ cells recovered was determined (FIG. 24). The percent CD19⁺ cells among the human CD45⁺ cells was determined (FIG. 25), and the percent GFP+ cells among the human CD19+ cells was determined (FIG. 26). The percent CD33⁺ cells among human CD45⁺ cells recovered from the bone marrow of NSGW41 mice engrafted with CD34+ cells cultured using protocol A or B was determined (FIG. 27), and the percent GFP+ cells among the human CD33+ cells was determined (FIG. 28). Representative flow plots of cells harvested from the bone marrow of NSGW41 mice at 16 weeks following transplant are shown in FIG. 29.

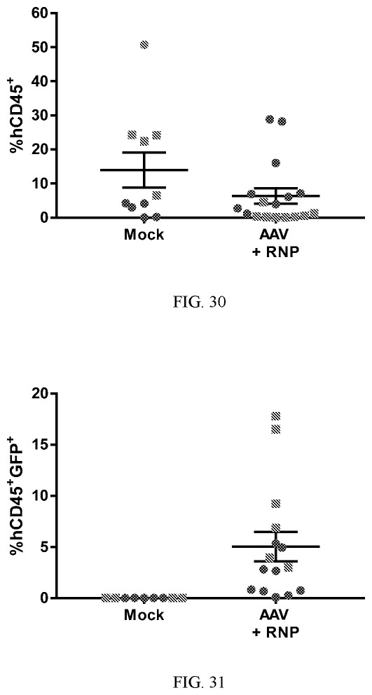

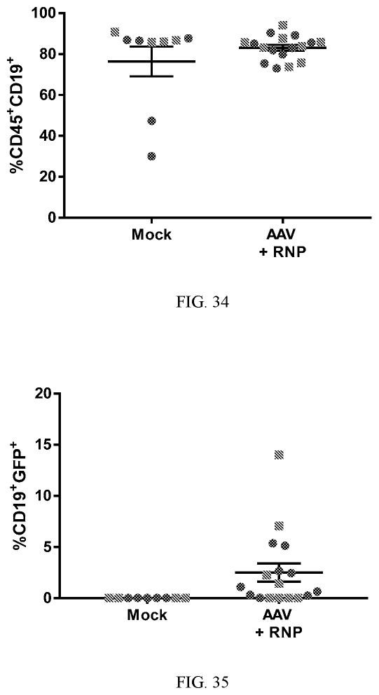

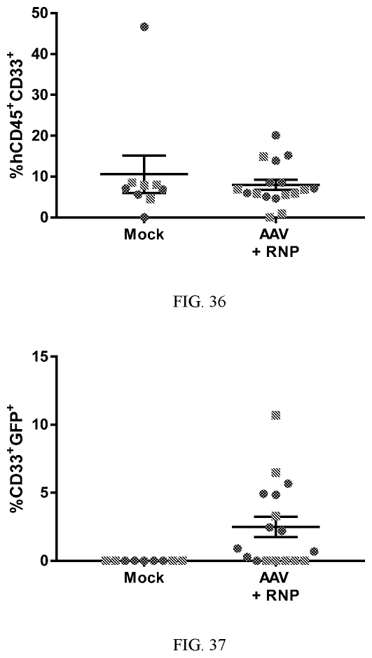

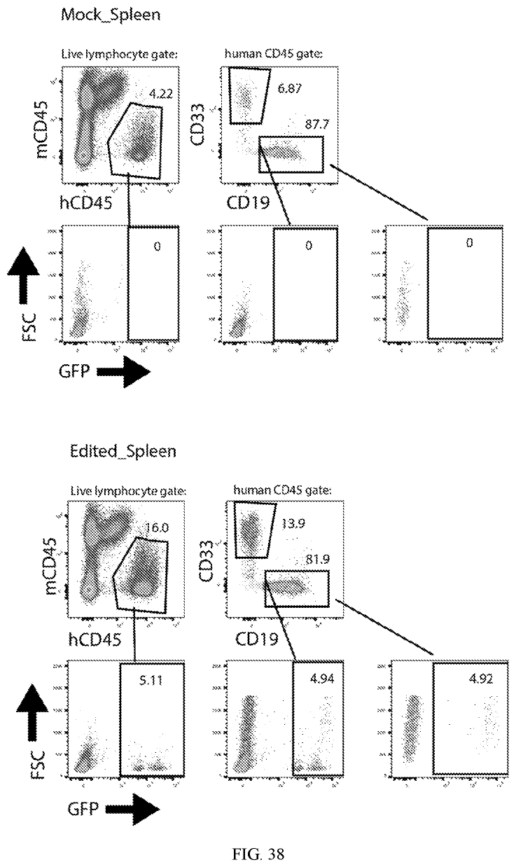

With regard to cells recovered from spleen, the percent human CD45⁺ cells recovered from the spleens of NSGW41 mice transplanted with CD34+ cells cultured using protocol A or B was determined (FIG. 30), and the percent GFP+ cells among the human CD45+ cells recovered was determined (FIG. 31). The percent CD19⁺ cells among human CD45⁺ cells recovered from the spleens of NSGW41 mice transplanted with CD34+ cells cultured using protocol A or B was determined (FIG. 34), and the percent GFP+ cells among the human CD19+ cells was determined (FIG. 35). The percent CD33⁺ cells among human CD45⁺ cells recovered from the spleens of NSGW41 mice transplanted with CD34+ cells cultured using protocol A or B was determined (FIG. 36), and the percent GFP+ cells among the human CD33+ cells was determined (FIG. 37). Representative flow plots of cells harvested from the spleens of NSGW41 mice 16 weeks following transplant are shown in FIG. 38.

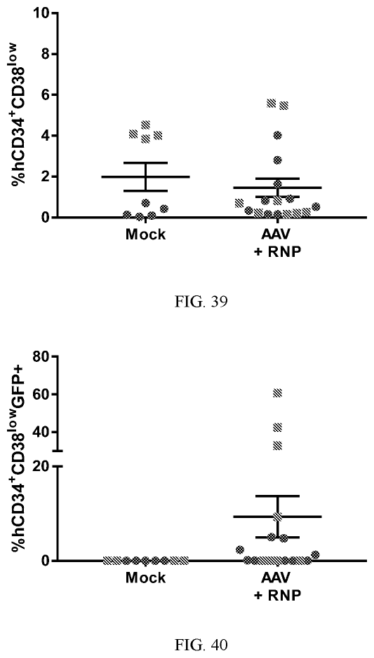

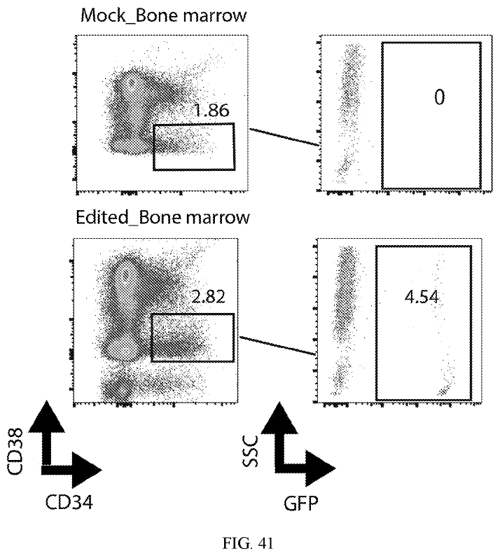

The percent CD34⁺CD38^{low} cells among human CD45⁺ cells recovered from the bone marrow of NSGW41 mice transplanted with CD34+ cells cultured using protocol A or B was determined (FIG. 39), and the percent GFP⁺ cells among the human CD34⁺CD38^{low} CD45⁺ cells was determined (FIG. 40). A representative flow cytometry analysis of CD34+ gated on total human CD45+ cells from NSGW41 mice transplanted with mock or edited cells is shown in FIG. 41 in which bone marrow cells from transplanted NSGW41 mice were harvested 16 weeks post-transplant and analyzed for the presence of LT-HSCs (long term repopulating hematopoietic stem cells). The LT-HSCs were characterized by low expression of CD38 and expression of CD34 markers. These cells were further analyzed for the presence of GFP+ cells indicating presence of edited cells within this population. GFP+ cells are present within this long-term HSC population demonstrating sustained HDR-editing of the CD40L locus in human HSC.

An increase in the engraftment of HDR-edited (GFP+) cells with both protocols (FIG. 24, FIG. 31) compared to prior studies. All recipient NSG animals harbored engrafted, HDR-edited human cells in both the myeloid and B cell populations in the bone marrow and spleen (FIG. 25, FIG. 27, FIG. 34, FIG. 36). These lineages were present at ratios equivalent to recipients of mock-edited human CD34+ cells. These data were consistent with editing of a multipotent HSC and indicated that that the differentiation capacity of HDR-edited stem cells was not compromised upon editing. HDR-edited (GFP+) cells were present in all cell lineages (B and myeloid) and were present in ratios equivalent to mock cells (FIG. 26, FIG. 28, FIG. 35, FIG. 37, FIG. 38). Notably, the percent of human CD45+ hematopoietic stem cells (HSC) engrafted within the bone marrow (as defined by expression of CD38low CD34+) was equivalent in mock and HDR-edited recipients (FIG. 39). GFP+ cells were present within this population consistent with editing of HSC capable of persisting long-term in vivo (FIG. 40, FIG. 41). The proportion of engrafted, HDR-edited HSC observed in these studies was consistent with a level predicted to provide clinical benefit following editing of autologous HSC from patients with CD40L deficiency. These combined findings indicated that this technology can provide clinical benefit for patients treated with this approach.

### Example 8—Co-Delivery with HDM2 Protein

HDM2 is an E3 ubiquitin-protein ligase that mediates ubiquitination of p53/TP53, leading to its degradation by the proteasome. The effect of co-delivery of HDM2 protein, along with RNPs plus AAV, on the rates of HDR observed in CD34+ cells was tested. Adult mobilized human CD34+ cells were cultured using protocol B for 48 hours followed by nucleofection of RNPs with or without HDM2 (6.25 nM-25 nM). AAV was added at the MOI of 1K. HDR rates were assessed by GFP expression on day 5.

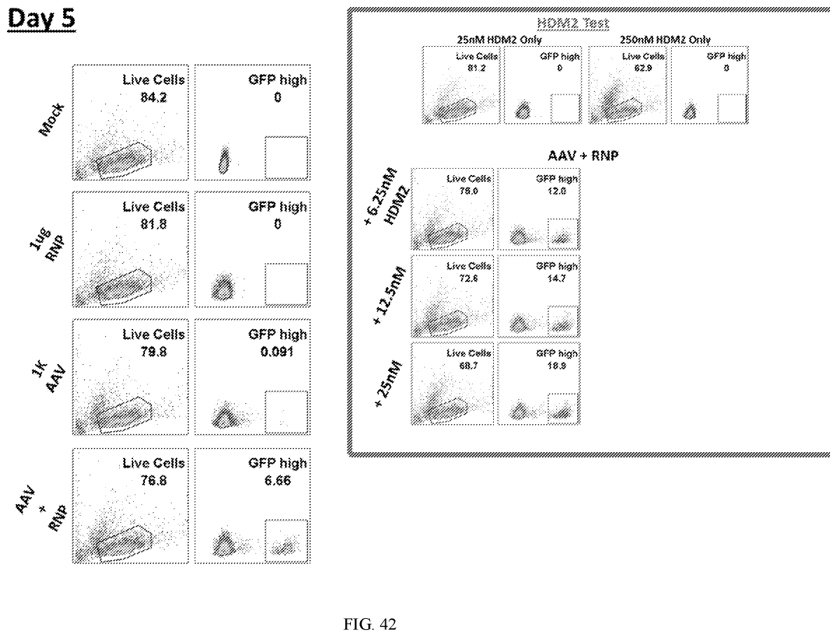

HDR rates were remarkably higher (up to 3-fold) for the cells co-transfected with the HDM2 protein compared to the one treated with only RNPs and AAV vector (FIG. 42). These data indicated that HDM2 can be utilized to improve HDR editing rates at the CD40L locus in CD34+ cells and can be utilized in conjunction with the above methods to increase HDR rates in long-term HSC.

It is to be understood that the description, specific examples and data, while indicating exemplary alternatives, are given by way of illustration and are not intended to limit the various alternatives of the present disclosure. Various changes and modifications within the present disclosure will become apparent to the skilled artisan from the description and data contained herein, and thus are considered part of the various alternatives of this disclosure.

1.-106. (canceled)

107. A method for editing an CD40LG gene in a cell, comprising:
(i) introducing a polynucleotide encoding a guide RNA (gRNA) into the cell, and
(ii) introducing a template polynucleotide into the cell, wherein the template polynucleotide encodes at least a portion of the CD40LG gene, or complement thereof.

108. The method of claim 107, wherein:
the gRNA comprises a nucleic acid having at least 95% identity to the nucleotide sequence of SEQ ID NO:12;
the template polynucleotide comprises a nucleic acid having at least 95% identity to the nucleotide sequence of SEQ ID NO:15; and/or
the CD40LG gene has at least 95% identity with the nucleotide sequence of SEQ ID NO:13.

109. The method of claim 107, wherein introducing a polynucleotide encoding a gRNA into the cell comprises contacting the cell with a ribonucleoprotein (RNP) comprising a CAS9 protein and the polynucleotide encoding the gRNA, wherein the CAS9 protein and the polynucleotide encoding the gRNA have a ratio between 0.1:1 and 1:10.

110. The method of claim 107, wherein steps (i) and/or (ii) comprise performing nucleofection, wherein performing nucleofection comprises use of a LONZA system, and wherein the system comprises use of a square wave pulse.

111. The method of claim 107, further comprising contacting the cell with:
IL-6, wherein the IL-6 has a concentration from about 20 ng/ml to about 500 mg/ml;
stem cell factor (SCF), FMS-like tyrosine kinase-3 (Flt-3), thrombopoietin (TPO), a TPO receptor agonist, UM171, or stemregenin (SR1); and/or
a SFEMII medium.

112. The method of claim 107, wherein a population of cells comprises the cell, the population having a concentration from about 1×10⁵ cells/ml to about 1×10⁶ cells/ml.

113. The method of claim 112, further comprising diluting the population of cells after steps (i) and (ii) are performed.

114. The method of claim 113, wherein the population of cells is diluted to about 250,000 cells/ml at about 16 hours after steps (i) and (ii) are performed.

115. The method of claim 107, wherein steps (i) and/or (ii) comprise contacting the cell with an HDM2 protein.

116. The method of claim 107, wherein an adeno-associated viral (AAV) vector comprises the template polynucleotide, and the cell is contacted with at least about 1000 MOI of the AAV; and/or wherein introducing a polynucleotide encoding a gRNA into the cell comprises contacting the cell with a ribonucleoprotein (RNP) comprising a CAS9 protein and the polynucleotide encoding the gRNA, and the cell is contacted with at least about 100 μg/ml of the RNP.

117. The method of claim 107, wherein steps (i) and/or (ii) comprise contacting about 1,000,000 cells/20 μl nucleofection reaction, wherein the nucleofection reaction comprises the gRNA and/or the template polynucleotide.

118. The method of claim 107, wherein the cell is selected from the group consisting of a hematopoietic stem cell (HSC), a T cell, a B cell, and a CD34+ cell.

119. A nucleic acid for homology directed repair (HDR) of CD40LG gene, the nucleic acid comprising:
a first sequence encoding at least a portion of a CD40LG gene;
a second sequence encoding one or more guide RNA cleavage sites; and
a third sequence encoding one or more nuclease binding sites.

120. The nucleic acid of claim 119, wherein:
the at least a portion of a CD40LG gene comprises at least a portion of the nucleotide sequence set forth in SEQ ID NO: 13;
the at least a portion of a CD40LG gene comprises at least about 1 kb of a CD40LG gene;
the second sequence comprises a nucleotide sequence having at least 95% identity with the nucleotide sequence set forth in SEQ ID NO: 12; and/or
the one or more nuclease binding sites comprises a forward and reverse transcription activator-like effector nuclease (TALEN) binding site, and/or a clustered regularly interspaced short palindromic repeats (CRISPR) associated protein 9 (Cas9) binding site.

121. A vector for promoting homology directed repair (HDR) of CD40L protein expression in a cell, the vector comprising the nucleic acid of claim 119.

122. The vector of claim 121, wherein the vector is an adeno-associated viral (AAV) vector.

123. A cell comprising the nucleic acid of claim 119.

124. The cell of claim 123, wherein the cell is selected from the group consisting of an autologous cell, a T cell, a hematopoietic stem cell (HSC), and a CD34⁺ cell.

125. A system for promoting homology directed repair (HDR) of CD40L protein expression in a cell, the system comprising the vector of claim 121, and a nucleic acid encoding a nuclease selected from a TALEN nuclease and a Cas nuclease.

126. A method of promoting homology directed repair (HDR) of a CD40LG gene in a subject in need thereof, the method comprising:
administering to a subject a cell or vector comprising the nucleic acid of claim 119; and
administering to the subject a nuclease selected from a TALEN nuclease and a Cas nuclease.

127. The method of claim 126, wherein the subject has an X-linked hyper IgM (X-HIGM) syndrome, and wherein the promoting HDR of a CD40LG gene in the subject treats, inhibits, or ameliorates the X-linked hyper IgM syndrome.
